(19) United States
(12) Patent Application Publication
(10) Pub. No.: US 2026/0079166 A1
(43) Pub. Date: Mar. 19, 2026

(54) METHODS FOR DETECTING FRACTURE-RELATED INFECTION (FRI)

(71) Applicants: THE TRUSTEES OF INDIANA UNIVERSITY, Bloomington, IN (US); PURDUE RESEARCH FOUNDATION, West Lafayette, IN (US); SAPIENZA UNIVERSITÀ DI ROMA, Rome (IT)

(72) Inventors: Roman M. NATOLI, Indianapolis, IN (US); Sarah MALEK, West Lafayette, IN (US); Federico Marini, Rome (IT)

(21) Appl. No.: 19/393,379

(22) Filed: Nov. 18, 2025

Related U.S. Application Data
(63) Continuation-in-part of application No. 18/698,129.
(60) Provisional application No. 63/256,394, filed on Oct. 15, 2021.

(51) Int. Cl. G01N 33/68; G01N 33/573; G01N 33/86

(52) U.S. Cl. CPC G01N 33/6893; G01N 33/573; G01N 33/6848; G01N 33/6872; G01N 33/86; G01N 2570/00; G01N 2800/26

(57) ABSTRACT

Methods of detecting diagnostic indicators of a fracture-related infection (FRI) in a patient's biological sample are provided along with methods of treating the identified patients having an FRI.

### RELATED APPLICATIONS

This application is a Continuation-In-Part of U.S. patent application Ser. No. 18/698,129, filed Apr. 3, 2024, which is a National Stage Entry of International PCT Application No. PCT/US2022/046515, filed Oct. 13, 2022, which claims the benefit of U.S. Provisional Application No. 63/256,394, filed Oct. 15, 2021. The entirety of each of which is hereby incorporated by reference.

### BACKGROUND

Fracture-related infection (FRI) is a severe complication following bone injury. The incidence of fracture-related infection (FRI) varies widely depending on the injury, but it is commonly reported as 5-10%. The cost of FRIs exceeds $23,000 per infection, and there are more than 20,000 FRIs annually in the United States. Despite the significant socio-economic impact, the ability to diagnose FRIs remains a challenge. The infection work-up is largely based upon the history and physical exam, white blood cell (WBC) count, erythrocyte sedimentation rate (ESR), C-reactive protein (CRP), radiographs, and occasionally advanced imaging. Unfortunately, these diagnostic tools are of limited utility. Quantitative histology and tissue culture from intra-operative tissue samples can be useful tools to diagnose FRIs, but they are invasive, dependent on sample quality, and the results are not available until after the surgery has been performed.

Historically, only 2% of randomized controlled trials defined an “infection”. To address this, an expert panel was assembled in 2017 to better define a FRI. Unfortunately, like many of the prosthetic joint infection definitions, the FRI definition is composed of confirmatory and suggestive criteria. Confirmatory criteria include a sinus tract communicating with the implant, purulent drainage, phenotypically indistinguishable pathogens from two deep tissue cultures, or the presence of microorganisms on histopathologic examination. Suggestive criteria include clinical and radiologic signs, elevated ESR, WBC and/or CRP, and non-purulent wound drainage.

There is limited utility of the currently used common biomarkers, namely WBC, ESR, and CRP, for diagnosing FRI. Additionally, only a few studies have evaluated other biomarkers, such as the cytokine IL-6. A large systematic review of 8,284 articles looking at the diagnostic accuracy of these “classic” serum inflammatory markers determined they are insufficient. In that review, sensitivity, and specificity for CRP ranged from 60-100% and 34-86%, respectively.

Accordingly, a need exists for a method capable of accurately detecting a fracture-related infection including biomarkers with high specificity and sensitivity and an algorithm that assesses and determines the concentration of biomarkers accurately.

### SUMMARY

The disclosure applies the discovery of a combination of biomarkers that accurately identify the presence of a fracture-related infection (FRI). FRIs are generally observed in patients after a surgery to introduce, replace, or adjust an implant. In one example, an FRI may occur after broken bones are re-set and stabilized using medical grade implants (e.g., bone plate and screws); also referred to “fracture fixation”. Historically, it is difficult to discern a fracture-related infection from inflammation and discomfort associated with recovery from surgery. The recommended process of identifying an FRI include blood sample analysis, imaging, performing biopsies from two separate locations, tissue culture, and histology analysis. All of these tests and analysis are required just to confirm an FRI before treatment options are discussed. Preventative measures have been added in the form of surgery sanitation protocols and antimicrobial coatings on implants; however, with the continued rise in antibiotic resistant microbes, these practices are not efficient to address the burdensome undertaking just to identify an FRI.

In one aspect, the disclosure provides a method of detecting an FRI in a subject comprising analyzing a blood sample and quantifying the concentration of proteins. In some embodiments, the proteins diagnostic for an FRI are selected from C-reactive protein (CRP), interleukin 6 (IL-6), platelet-derived growth factor AB BB (PDGF-AB BB), and vascular endothelial growth factor A (VEGF-A), or a combination thereof. In some embodiments, the proteins diagnostic for an FRI include two or more proteins selected from CRP, IL-6, PDGF-AB BB, and VEGF-A. In some embodiments, the proteins diagnostic for an FRI are selected from interleukin 6 (IL-6), platelet-derived growth factor AB BB (PDGF-AB BB), and vascular endothelial growth factor A (VEGF-A), optionally in combination with C-reactive protein (CRP) or other combination thereof. In some embodiments, the proteins diagnostic for an FRI include the proteins CRP, IL-6, PDGF-AB BB, and VEGF-A, optionally in combination with CRP.

In another aspect, the disclosure provides a method of detecting an FRI by obtaining a spectral profile and comparing the spectral peaks to a model/control to detect the presence of a spectral pattern associated with an FRI.

In one embodiment a method of treating an FRI is provided, once an FRI has been identified using one of the diagnostic methods disclosed herein. In one embodiment the treatment comprises using antimicrobial therapies known to the skilled practitioner, including for example the use of one or more antibiotics.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph showing the plasma concentration of C-reactive protein (CRP) differences between the confirmed infection group and control group.

FIG. 2 is a graph showing the plasma concentration of interleukin-6 (IL-6) differences between the confirmed infection group and control group.

FIG. 3 is a graph showing the plasma concentration of platelet-derived growth factor (PDGF)-AB BB differences between the confirmed infection group and control group.

FIG. 4 is a graph showing the plasma concentration of vascular endothelial growth factor (VEGF)-A differences between the confirmed infection group and control group.

FIG. 5 is a graph showing the Receiver Operator Characteristics (ROC) of CRP.

FIG. 6 is a graph showing the ROC of IL-6.

FIG. 7 is a graph showing the ROC of PDGF-AB BB.

FIG. 8 is a graph showing the ROC of VEGF-A.

FIG. 9 is a diagram showing the workflow of analytical steps for spectral data and ELISA-based measured values of plasma samples from FRI and control groups.

FIG. 10 is a graph showing an ROC curve for enzyme-linked immunosorbent assay (ELISA)-based biomarkers, wherein the ROC curve was calculated based on predictions from the reporting of the repeated (×50) double-cross-validation (rDCV) outer loops (both the ones corresponding to the individual DCV runs and their average (thick dark line)). X axis is 1-specificity (false positive rate), and Y axis is sensitivity (false negative rate).

FIG. 11 is a graph showing an ROC curve for Fourier-transform infrared (FTIR) spectral data, wherein the ROC curve was calculated based on predictions from the reporting of the repeated (×50) double-cross-validation (rDCV) outer loops (both the ones corresponding to the individual DCV runs and their average (thick dark line)). X axis is 1-specificity (false positive rate), and Y axis is sensitivity (false negative rate).

FIG. 12 is a graph showing an ROC curve when including both ELISA-based biomarkers and FTIR spectral data, wherein the ROC curve was calculated based on predictions from the reporting of the repeated (×50) double-cross-validation (rDCV) outer loops (both the ones corresponding to the individual DCV runs and their average (thick dark line)). X axis is 1-specificity (false positive rate), and Y axis is sensitivity (false negative rate).

FIG. 13 is a graph showing results of covariance selection-partial least squares-discriminant analysis (CovSel-PLS-DA) modeling on ELISA-based protein biomarker data, wherein double-cross-validated projections of the outer loop samples onto the only canonical variate of the classification model shows the difference in the values of the scores (bars indicate mean and whiskers the corresponding 95% confidence intervals) between FRI and control patients' samples.

FIG. 14 is a graph showing results of CovSel-PLS-DA modeling on ELISA-based protein biomarker data and the weights of the selected markers for the construction of that projection direction (canonical variate). FRI, fracture related infection group; Ctrl, control group; PDGF-AB/BB, platelet-derived growth factor AB/BB; CRP, C-reactive protein; MIG, monokine induced by gamma interferon.

FIG. 15 is a graph showing the results of CovSel-PLS-DA modeling on IR spectroscopic data and identifies the six variables (wavenumbers) selected as relevant by the CovSel algorithm (3288.7, 1648.6, 1624.3, 1592.9, 1188.2 and 610.6 cm-1; red vertical bars) over the mean IR spectrum of the samples.

FIG. 16 is a graph showing the results of CovSel-PLS-DA modeling on IR spectroscopic data, and the double-cross-validated projections of the outer loop samples onto the only canonical variate of the classification model showing the difference in the values of the scores (bars indicate mean and whiskers the corresponding 95% confidence intervals) between FRI and control patient samples.

FIG. 17 is a graph showing the results of CovSel-PLS-DA modeling on IR spectroscopic data, and the weights of the selected markers for the construction of that projection direction (canonical variate). FRI, fracture related infection group; Ctrl, control group.

FIG. 18 is a graph showing results of PLS-DA data fusion of selected ELISA-based biomarkers and FTIR spectral variables, and the double-cross-validated projections of the outer loop samples onto the only canonical variate of the classification model showing the difference in the values of the scores (bars indicate mean and whiskers indicate the corresponding 95% confidence intervals) between FRI and control patients' samples.

FIG. 19 is a graph showing results of PLS-DA data fusion of selected ELISA-based biomarkers and FTIR spectral variables and the weights of the selected markers for the construction of that projection direction (canonical variate). FRI, fracture related infection group; Ctrl, control group; CV, coefficient variable; PDGF-AB/BB, platelet-derived growth factor AB/BB; CRP, C-reactive protein; MIG, monokine induced by gamma interferon.

FIG. 20 is an exemplary diagram showing the diagnostic algorithm from the international FRI Consensus Group, publicly available from (Metsemakers, Morgenstern, McNally, Moriarty, McFadyen, Scarborough, Athanasou et al. 2018).

FIG. 21 is an exemplary table showing the Patient Demographics and Relevant Fracture Characteristics. Values expressed as means (S.D.) for continuous data and counts (proportion) for categorical data. P-values from two-tailed t-test for continuous data and Chi-square test for categorical data.

FIG. 22 shows the Random Forest Classification using all detected metabolites in plasma of matched Control samples compared to Infection samples, which gave a predictive accuracy of 79.7%, and shows exemplary results from random forest analysis including predictive accuracy and importance plot of the top 30 biochemicals (i.e., biomarkers) which contributed the most (by means decrease accuracy) to this model.

FIG. 23 is an exemplary diagram showing the preparation of technical replicates. A small aliquot of each client sample was pooled to create a CMTRX technical replicate sample, which was then injected periodically throughout the platform run. Variability among consistently detected biochemicals was used to calculate an estimate of overall process and platform variability.

FIG. 24 shows the visualization of data normalization steps for a multiday platform run.

FIG. 25 is an exemplary diagram showing the diagnostic algorithm for a patient suspected of fracture-related infection (FRI). The algorithm is based on the updated definition of FRI ESR (erythrocyte sedimentation rate), WBC (white blood cell count), CRP (C-reactive protein).

FIGS. 26A-C are exemplary FTIR spectra visualizations showing the normalized averaged FTIR spectra of the control samples (A) and the FRI samples (B), along with the log ratio of the averaged spectra (C), highlighting differences in the FTIR signal. Arbitrary units (a.u.)

FIG. 27 shows the predictive model performance for FTIR spectra. Bootstrap estimation of AUROC, sensitivity and specificity of the elastic net classifier trained on the DC-transformed data.

FIG. 28 shows the separation of the control and FRI samples by FTIR spectra. Projection of the transformed, feature-filtered, and SVD-compressed FTIR spectra onto two discriminant coordinates illustrated the separation between control and FRI and control samples. The ellipses represent the 95-percentile confidence boundaries for the data groups, calculated based on a multivariate t-distribution.

FIG. 29 shows an exemplary predictive model performance for mass spectrometry (MS) data. Bootstrap estimation of AUROC, sensitivity, and specificity of the elastic net classifier trained on the MS data.

FIG. 30 is an exemplary visualization of the study data in box plot format and the legend for plots used. Box plots were used to convey the spread of the data with the middle 50% of the data represented by the shaded boxes and the whiskers reporting the range of the data. The solid bar across the box represents the median value of those measured while the + represents the mean. Data are scaled such that the median value measured across all samples was set to 1.0. Any outliers are shown as dots outside the whiskers of the plot.

FIG. 31 shows an exemplary Principal Component Analysis (PCA) of plasma samples, color coded by experimental group or pairing designation.

FIG. 32 shows an exemplary statistical heat map, pathway diagram, and boxplot of select metabolites associated with free fatty acid metabolism. In the heatmap, trending (0.05<p<0.10) and significant (p≤0.05) elevations are indicated by pink and red, respectively, while trending and significant reductions are represented by light green and dark green, respectively.

FIG. 33 shows an exemplary statistical heat map, pathway diagram, and boxplot of select metabolites associated with phospholipid metabolism. In the heatmap, trending (0.05<p<0.10) and significant (p<0.05) elevations are indicated by pink and red, respectively, while trending and significant reductions are represented by light green and dark green, respectively.

FIG. 34 shows an exemplary statistical heat map, pathway diagram, and boxplot of select metabolites associated with benzoate metabolism. In the heatmap, trending (0.05<p<0.10) and significant (p≤0.05) elevations are indicated by pink and red, respectively, while trending and significant reductions are represented by light green and dark green, respectively.

FIG. 35 shows an exemplary statistical heat map, pathway diagram, and boxplot of select metabolites associated with primary bile acid metabolism. In the heatmap, trending (0.05<p<0.10) and significant (p≤0.05) elevations are indicated by pink and red, respectively, while trending and significant reductions are represented by light green and dark green, respectively.

FIG. 36 shows the results of the 1,453 metabolites analyzed. Of the 1,453 metabolites analyzed, 1,182 were identified and 271 unnamed. A full list of the metabolites, organized by biochemical class, is shown. Matched pairs t-testing identified 248 significantly different metabolites between FRI and control serum (103 upregulated, 145 downregulated). Of these, 83 had a q-value less than 0.05.

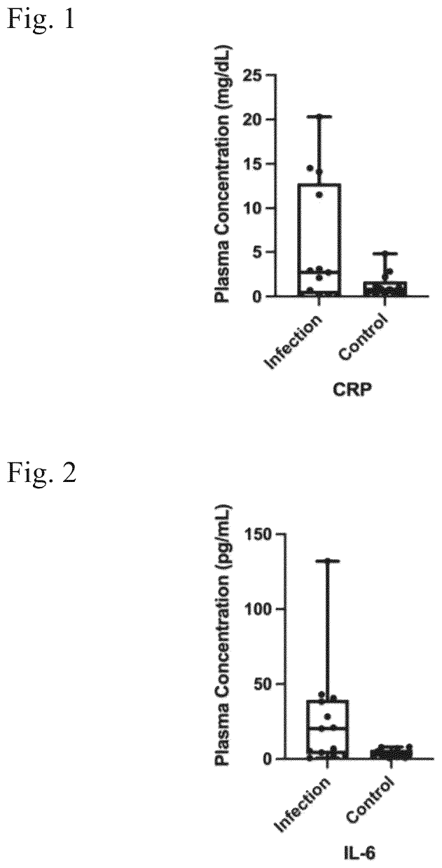

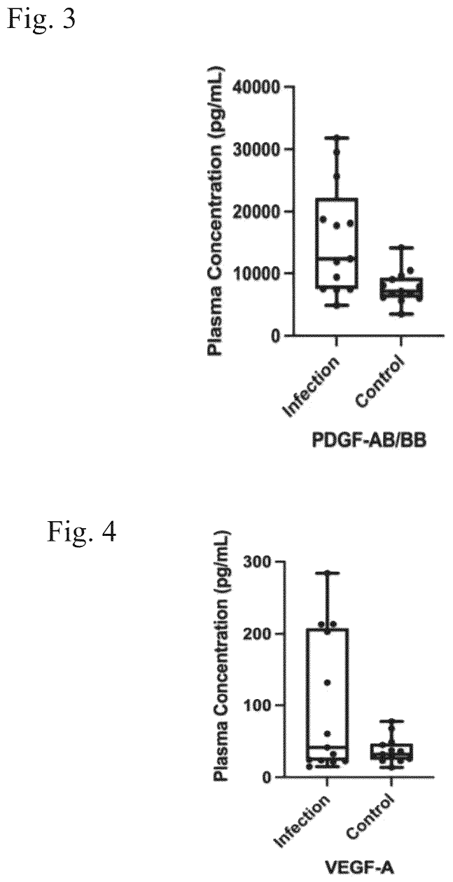

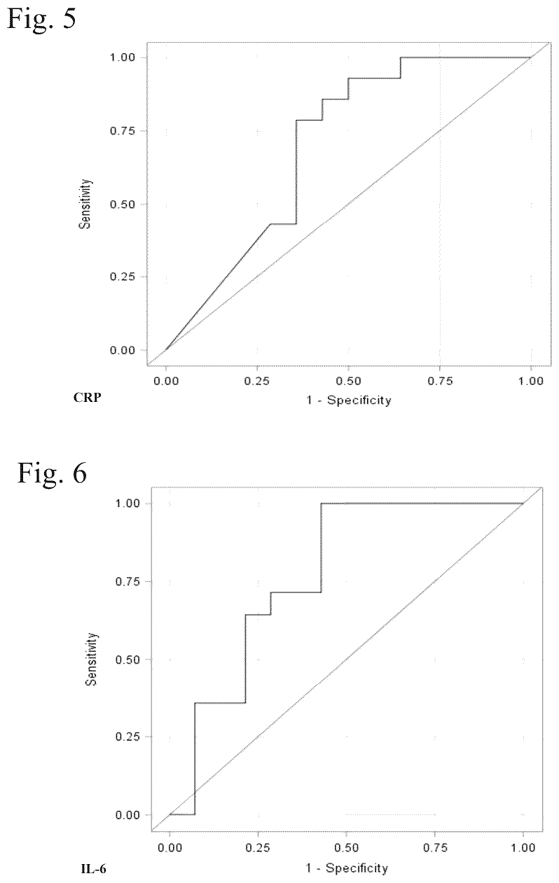

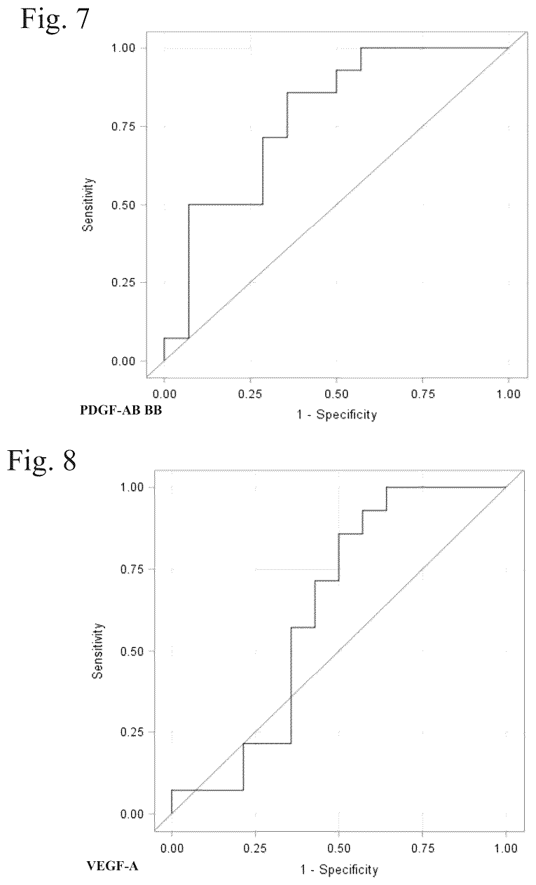

### DETAILED DESCRIPTION

### Abbreviations

FRI stands for fracture-related infection.
ROC stands for receiver operator characteristics.
AUC stands for area under the curve.
WBC stands for white blood cell.
CRP stands for C-reactive protein.
ESR stands for erythrocyte sedimentation rate.
IL-6 stands for interleukin 6.
PDGF-AB BB stands for platelet-derived growth factor AB BB.

VEGF-A stands for vascular endothelial growth factor A.

PJI stands for periprosthetic joint infection.

MIG stands for monokine induced by gamma interferon.

### Definitions

In describing and claiming the methods, the following terminology will be used in accordance with the definitions set forth below.

The term “effective” amount or a “therapeutically effective amount” of a compound refers to a nontoxic but sufficient amount of the compound to provide the desired effect. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term “cut point” defines a threshold concentration that indicates the likely presence of an FRI. In some aspects, the cut point is used to optimize the detection and concentration of the protein biomarkers, e.g., in analysis of an ELISA assay.

The term “subject” or “patient” means an animal including, but not limited to, humans, domesticated animals including horses, dogs, cats, cattle, and the like, rodents, reptiles, and amphibians receiving a therapeutic treatment either with or without physician oversight. In some respects, an animal may be referred to as a subject or a patient. In some embodiments, the subject is a post-operative patient having undergone surgery to introduce, replace, or adjust an implant.

The term “fracture” a used herein refers to any injury or breakage of bones and includes damage to bones ranging from small hairline fractures to traumatic bone breaks.

The term “fracture-related infection (FRI)” encompasses all infections which occur in the presence of a fracture or the introduction, replacement, or adjustment of an implant. This includes early infection around fracture implants, infected non-unions, haematogenous infections arising after fracture healing and infections in fractures with no internal fixation as well as infections associated with implants.

### EMBODIMENTS

Embodiments of the disclosure include a method of detecting an FRI and a system for detecting an FRI using antibodies, spectroscopic profile(s), or both as well as methods for treating patients who have been identified as having an FRI.

The method includes the detection of FRIs occurring on any part of the skeleton. The fracture associated with the FRI may include one or more fractures of the same bone or cartilage. Alternatively, there may be more than one fracture on more than one bone and/or cartilage.

In some aspects, the present disclosure provides a method of treating a fracture-related infection (FRI) in a subject having FRI, said method comprising:
analyzing a subject's biological sample using an analytical method to determine whether a subject has an FRI; and
administering an anti-infection therapy to said subject identified as having an FRI.

In some embodiments, the step of analyzing a subject's biological sample comprises:
obtaining an analytical patient profile of a biological sample obtained from the subject; and
comparing the analytical patient profile to a reference analytical patient profile.

In some embodiments, the step of obtaining an analytical patient profile further comprises a step of diluting a biological sample with potassium thiocyanate.

In some embodiments, the step of comparing the analytical patient profile to a reference analytical patient profile comprises determining if the biological sample comprises an increased amount or a decreased amount of a biomarker compared to a reference analytical patient profile. In some embodiments, the biological sample comprises an increased amount of a biomarker compared to a reference analytical patient profile. In some embodiments, the biological sample comprises a decreased amount of a biomarker compared to a reference analytical patient profile.

In some embodiments, the analytical method provides an analytical patient profile.

In some embodiments, the analytical method comprises a mass spectrometer. In some embodiments, the analytical method comprises Tandem mass tag liquid chromatography-mass spectrometry (TMT LC-MS/MS), a reverse phase ultrahigh performance liquid chromatography-tandem mass spectroscopy (RP)/UPLC-MS/MS method using positive ion mode electrospray ionization (ESI), or a (RP)/UPLC-MS/MS method using negative ion mode ESI.

In some embodiments, the analytical method comprises a Fourier-transform infrared spectroscopy (FTIR) spectrometer.

In some embodiments, the subject is a human. In some embodiments, the subject is an adult.

In some embodiments, the FRI is associated with a surgical implant. In some embodiments, the FRI comprises an infection which occurs in the presence of a fracture. In some embodiments, the FRI is associated with the introduction of an implant, the replacement of an implant, or the adjustment of an implant. In some embodiments, the FRI comprises an infection that occurs in the presence of the introduction, replacement, or adjustment of an implant.

In some embodiments, the FRI is caused by one or more pathogens. In some embodiments, the FRI is caused by more than one pathogen.

In some embodiments, the pathogen comprises a Gram-positive bacterium, a Gram-negative bacterium, an anaerobic bacterium, a mycobacterium, a fungus, or any combination thereof.

In some embodiments, the pathogen comprises a Gram-positive bacterium. In some embodiments, the Gram-positive bacterium comprises a Gram-positive cocci. In some embodiments, the Gram-positive bacterium comprises a Gram-positive rod. In some embodiments, the Gram-positive bacterium comprises one or more Staph species. In some embodiments, the Gram-positive bacterium comprises Staphylococcus aureus, Staphylococcus epidermidis, or a combination thereof.

In some embodiments, the pathogen comprises a Gram-negative bacterium. In some embodiments, the Gram-negative bacterium comprises a Gram-negative cocci. In some embodiments, wherein the Gram-negative bacterium comprises Pseudomonas aeruginosa, Escherichia coli, or a combination thereof.

In some embodiments, the Gram-positive cocci comprises MRSA, MSSA, a Coagulase-negative staphylococci (CoNS), B Hemolytic Streptococcus (Strep) Group A, B Hemolytic Strep Group B, Hemolytic Strep Group C, Hemolytic Strep Group G, one or more Enterococcus species, Staphylococcus (Staph) Lugdenesis, Staph epidermidis, Staph capitis, Strep Pneumoniae, viridans Strep, nutritionally variant streptococci (NVS), one or more Finegoldia species, one or more Micrococcus species, Granulicatella, one or more Staph species excluding Staph Aureus, or any combination thereof. In some embodiments, the Gram-positive cocci comprises one or more Staph species.

In some embodiments, the Gram-negative cocci comprises one or more Acinetobacter species, one or more Aeromonas species, one or more Brucella species, one or more Chyrseobacterim species, one or more Citrobacter species, one or more E Coli species, one or more Eikenella species, one or more Enterobacter species, one or more Fusobacterium species, one or more Klebsiella species, one or more Pantoea species, one or more Pasteurella species, one or more Proteus species, one or more Pseudomonas species, one or more Serratia species, one or more Stenotrophomonas species, or one or more Wolinella species.

In some embodiments, the Gram-positive rod comprises one or more Bacillus species, one or more Corynebacterium species, or one or more Dermabacter species.

In some embodiments, the fungus comprises one or more Trichophyton species or one or more Candida species.

In some embodiments, the anerobic bacterium comprises one or more Anaerococcus species, one or more Bacteroides species, one or more Clostridium species, one or more Helcoccus species, one or more Parabacteriodes species, one or more Peptoniphilus species, one or more Peptostreptococcus species, one or more Porphyromonas species, or one or more Prevotella species.

In some embodiments, the anti-infection therapy comprises an anti-infective.

In some embodiments, the anti-infective comprises a cephalosporin, linezolid, dalbavancin, metronidazole, a fluoroquinoline, gentamicin, tobramycin, vancomycin, clindamycin, an antifungal, or any combination thereof. In some embodiments, the anti-infective comprises cefazolin.

In some embodiments, the anti-infection therapy comprises administering an anti-infective for between about 2 weeks and about 12 weeks. In some embodiments, the anti-infection therapy comprises administering an anti-infective for between about 4 weeks and about 10 weeks. In some embodiments, the anti-infection therapy comprises administering an anti-infective for between about 3 weeks and about 6 weeks. In some embodiments, the anti-infection therapy comprises administering an anti-infective for about 2 weeks. In some embodiments, the anti-infection therapy comprises administering an anti-infective for about 3 weeks. In some embodiments, the anti-infection therapy comprises administering an anti-infective for about 4 weeks. In some embodiments, the anti-infection therapy comprises administering an anti-infective for about 5 weeks. In some embodiments, the anti-infection therapy comprises administering an anti-infective for about 6 weeks. In some embodiments, the anti-infection therapy comprises administering an anti-infective for about 7 weeks. In some embodiments, the anti-infection therapy comprises administering an anti-infective for about 8 weeks. In some embodiments, the anti-infection therapy comprises administering an anti-infective for about 9 weeks. In some embodiments, the anti-infection therapy comprises administering an anti-infective for about 10 weeks. In some embodiments, the anti-infection therapy comprises administering an anti-infective for about 11 weeks. In some embodiments, the anti-infection therapy comprises administering an anti-infective for about 12 weeks.

In some embodiments, the biological sample comprises blood, urine, a synovial fluid, or any combination thereof. In some embodiments, the biological sample comprises blood, urine, or a combination thereof. In some embodiments, the biological sample comprises blood plasma.

In some embodiments, the biological sample comprises a biomarker.

In some embodiments, the biomarker comprises a protein, a non-protein metabolite, a xenobiotic, heme, a nucleic acid, or any combination thereof. In some embodiments, the biomarker comprises a lipid, a sugar, a beta hydroxy acid, an amino acid, a bile acid, or a derivative thereof.

In some embodiments, the biomarker comprises myristoleate, 14-methylpalmitate, 15-methylpalmitate, 16-hydroxypalmitate, 10-undecenoate, palmitoleate, 12-methylmyristate, 13-methylmyristate, 5-dodecenoate, pentadecanoate, myristate, glycholate, hexadecadienoate, eicosapentaenoate, hexadecanedioate, 1-linoleoyl-2-arachidonoyl-GPC (18:2/20:4n6), 1-palmitoyl-2-arachidonoyl-GPI, -linolenoyl-GPC (18:3), or any combination thereof.

In some embodiments, the amino acid or a derivative thereof comprises alanine, N-lactoyl-isoleucine, N-lactoyl-tyrosine, N-acetyl-glutamide, methionine sulfoxide, or any combination thereof.

In some embodiments, the biomarker comprises benzoate, vanilloylglycine, 3-hydroxy-2-methylpyridine sulfate, hippurate, 2-hydroxyhippurate (salicylurate), levulinate (4-oxovalerate), ferulic acid 4-sulfate, vanillic alcohol, a derivative thereof, or any combination thereof.

In some embodiments, the beta hydroxy acid comprises 3-hydroxybutyrate (BHBA).

In some embodiments, the bile acid comprises glycocholate, glycochenodeoxycholate, taurochenodeoxycholate, or any combination thereof.

In some embodiments, the nucleic acid comprises deoxyribonucleic acid (DNA), ribonucleic acid (RNA), or a combination thereof. In some embodiments, the RNA comprises microRNA.

In some embodiments, the biomarker comprises alpha-1-acid glycoprotein 1, serum amyloid A-2 protein, leucine-rich alpha-2-glycoprotein, complement factor H-related protein 5, C-reactive protein (CRP), complement component C9, haptoglobin, serum amyloid A-1 protein, lipopolysaccharide-binding protein, peptidyl-prolyl cis-trans isomerase A, calmodulin-3, fructose-bisphosphate aldolase A, complement factor B, peroxiredoxin-2, mannosyl-oligosaccharide 1,2-alpha-mannosidase IA, alpha-1-acid glycoprotein 2, Transgelin-2, complement C1r subcomponent, retinoic acid receptor responder protein 2, ceruloplasmin, complement C1s subcomponent, carboxypeptidase N catalytic chain, alpha-2-macroglobulin, coagulation factor X, Insulin-like growth factor-binding protein 4, selenoprotein P (SEPP1), fibronectin, cell surface glycoprotein MUC18, hepatocyte growth factor activator, apolipoprotein A-IV, plasma kallikrein, afamin, platelet-derived growth factor AB/BB (PDGF-AB/BB), monokine induced by gamma interferon (MIG), Interleukin 6 (IL-6), vascular endothelial growth factor A (VEGF-A), fibrin, procalcitonin, neutrophil CD64 (nCD64), CD66b, or any combination thereof. In some embodiments, the biomarker comprises CRP, PDGF-AB/BB, MIG, IL-6, or VEGF-A.

To streamline and reduce the number of tests required to diagnose a FRI, the disclosure provides a method of detecting and analyzing protein biomarkers with specificity for FRI. In some embodiments, it is envisioned that the financial cost and time to diagnose an FRI will be significantly reduced.

In one illustrative aspect, a method is provided for detecting the presence of an FRI. The method includes detecting and analyzing protein biomarkers in a sample obtained from a subject. The protein biomarkers may be produced during or cause an inflammatory response. The protein biomarkers may be obtained from any biological sample recovered from the patient including urine or blood. In one embodiment the biological sample is a whole blood, serum or plasma sample.

The subject may or may not be suspected of having an FRI. The subject may have a permanent or temporary implant. The subject may have undergone fracture fixation surgery to repair at least one fracture on a bone of the subject. The subject may have had surgery to repair multiple fractures on one or more bones. The fracture may be open or closed.

The subject's bone may be any bone in the human skeleton. The bone may be homologous or heterologous.

In one aspect, the method includes the detection of a biomarker above or below a certain concentration threshold. The concentration threshold is relative to a matching subject that does not have an FRI. In some embodiments, a biomarker detected in a subject sample at a concentration higher than that of a control sample would indicate that the subject has FRI. The method may include the detection of at least one, at least two, at least three, or at least four protein biomarkers. Each biomarker may be detected at a concentration threshold higher or lower than a control concentration. For example, where three biomarkers are detected and two are detected at a lower concentration thresholds than the control concentrations, the subject's probability of not having an FRI is more likely than if only one of the three biomarkers had a concentration detected at or below a concentration threshold. Having three of the diagnostic proteins of the present disclosure detected below the respective threshold values reliably predicts no FRI better than detecting only two below their respective threshold values. When all four are below the respective threshold, specificity is 100%.

In some aspects where at least four of the diagnostic proteins of the present disclosure are selected to be detected in a subject's sample, the detection of at least one, at least two, at least three, or at least four of the protein biomarkers at a concentration at or higher than each biomarker's concentration threshold would indicate the presence of FRI. When the method is designed to detect one biomarker above the thresholds, the sensitivity of diagnosis is about 85% for the presence of FRI.

The method may include the detection of a biomarker in a subject's sample and analyzing and determining the concentration of that biomarker. In some embodiments, the biomarker is selected from PDGF-AB BB, VEGF-A, IL-6, CRP, MIG, or a combination thereof. The method may include the detection of PDGF-AB BB and at least one other biomarker selected from VEGF-A, IL-6, MIG, or CRP. In other aspects, the method may include the detection of PDGF-AB BB, VEGF-A, IL-6, MIG and CRP. The method may include the detection of PDGF-AB BB, CRP, and MIG. Additionally, the method includes analyzing and determining the concentration of the biomarker. The concentration is then referenced to the cut-point, to determine the probability of FRI.

In one embodiment, the method includes detection of PDGF-AB BB from a sample derived from a subject. The detection of PDGF-AB BB at a concentration at or above about 12,000 pg/mL, about 11,500 pg/mL, about 11,000 pg/mL, about 10,500 pg/ml, or about 10,000 pg/mL indicates the presence of a FRI. In another aspect the cut-point is at or below about 10,550 pg/mL, about 10,500 pg/mL, about 10,450 pg/mL, about 10,400, or about 10, 350 pg/mL. The cut-point may be about 10, 445 pg/mL, about 10, 444 pg/mL, about 10,443 pg/mL, about 10,442 pg/mL, about 10,441 pg/mL, or about 10,440 pg/mL.

The method may include detecting VEGF-A from a sample derived from a subject. The detection of VEGF-A at a concentration at or above about 80 pg/mL, 79.5 pg/mL, 79 pg/mL, 78.5 pg/mL, 78 pg/mL, 77.5 pg/mL, 77 pg/mL, 76.5 pg/mL, 76 pg/mL or 75.5 pg/mL indicates the presence of an FRI.

The method may include detecting IL-6 from a sample derived from a subject. The detection of IL-6 at a concentration at or below about 8.2 pg/mL, 8.1 pg/mL, 8.0 pg/mL, 7.9 pg/mL, 7.8 pg/mL, 7.7 pg/mL, 7.6 pg/mL, or 7.5 pg/mL indicates the presence of an FRI.

The method may include detecting CRP from a sample derived from a subject. The detection of CRP at a concentration at or below about 3.1 mg/dL, 3.0 mg/dL, 2.9 mg/dL, 2.8 mg/dL, 2.7 mg/dL, 2.6 mg/dL, or 2.5 mg/dL indicates the presence of an FRI.

The plasma or whole blood sample may be obtained from a subject up to six months after initial surgery to repair the fracture. In some instances, the sample may be obtained between about 1 day to about 6 months after fracture fixation surgery. In some instances, the sample is obtained 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 28 day, 32 days, two months, three months, four months, or five months after fracture fixation surgery. In one embodiment the sample is obtained at about 1 week, about 2 weeks, about 3 weeks, 4 weeks, about 5 weeks, or about 6 weeks after fracture fixation surgery. The sample may be fresh or frozen and thawed prior to analysis.

The protein biomarkers may be detected and analyzed using an antibody-based assay, such as an ELISA or any other detection method known to those skilled in the art. In accordance with one embodiment a kit for detecting a FRI is provided, wherein the kit comprises antibodies specific for each of PDGF-AB BB, VEGF-A, IL-6, and CRP. In one embodiment the kit further comprises an antibody specific for MIG. In one embodiment the antibodies are monoclonal antibodies. In one embodiment the antibodies are labeled with a detectable marker. In one embodiment the antibodies are covalently linked to a solid support.

In accordance with one embodiment a profile of a patient's biological sample (e.g., a urine, serum or plasma sample) as determined by spectroscopic analysis can be used to detect the presence of an FRI. In one embodiment a method for obtaining a mid-infrared (MIR) spectroscopic profile of a plasma sample (or other biological fluid recovered from a patient) using Fourier-transform infrared spectroscopy (FTIR) is provided. The method comprises acquiring FTIR spectra based on measurements of plasma samples using a dried film technique. In this method the plasma is dried on a microplate that is then read by the machine (FTIR spectrometer) and the spectral pattern is displayed in form of a unique waveform. This waveform (i.e., spectrum) undergoes preprocessing before the analytical modeling is conducted. Preprocessing involves a variety of steps that reduces redundant information and noise from the spectra (e.g., scatter correction and derivative techniques). Multivariate analytical methods are needed for comparing spectra and development of predictive models. The predictive model algorithms based on spectra can identify features that are unique to the disease state (i.e., FRI) compared to controls. The MIR spectroscopic profile may be referred to as a “spectral biomarker”, “biochemical fingerprint,” a “spectral fingerprint,” or simply a “fingerprint.”

In a further aspect, there is provided a system for obtaining a serum MIR spectroscopic profile using Fourier-transform infrared spectroscopy (FTIR). In one embodiment the system comprises one or more of a FTIR spectrometer, a preprocessing module, a normalization module, and a user interface. The FTIR spectrometer is used for obtaining FTIR spectra from the plasma samples. The preprocessing module may preprocess the FTIR spectra by differentiation and smoothing to enhance weak spectral features and to remove baseline variations, or other validated methods. In the development phase, the user interface utilizes the analytical methods for spectral analysis to develop the predictive model algorithms based on these spectra to identify FRI spectra from control healthy spectra. These developed predictive algorithms are then embodied in the form of a software that would read spectra from new plasma samples and classify them as FRI versus control based on their “spectral fingerprint”.

In an embodiment, the system further comprises a pattern recognition module for identifying, in the serum spectroscopic profile, spectroscopic features conveying diagnostic information of interest using pattern recognition models and a diagnostic module for diagnosing a fracture-related infection.

In a further aspect, there is provided a machine-readable medium containing sets of instructions, code sequences, configuration information, or other data, which, when executed, cause a processor to perform steps in a method for obtaining a serum mid-IR spectroscopic profile using FTIR. The method comprises acquiring FTIR spectra for dried plasma and preprocessing the FTIR spectra. The preprocessed FTIR spectra are normalized to a common intensity range, the normalization being performed in a spectral sub-region defined by strongest IR absorption for a protein to obtain the serum spectroscopic profile for providing a basis to diagnose an FRI.

For diagnostic analysis, a pattern recognition technique may be used to seek specific spectral ranges within which the spectra differed for normal specimens and those having an FRI. The pattern recognition model is optimized using the predictive algorithms disclosed and exemplified herein. Generally, the predictive algorithms were developed using spectral data obtained from FTIR spectroscopy performed on samples from confirmed FRI and control samples. The algorithms were designed to be implemented on spectrometers (portable or stationary) to detect the spectral data indicative of an FRI.

In one aspect, the pattern recognition model disclosed herein identified up to six wavenumbers of interest to differentiate between an FRI patient and a matching control patient. The method may include the detection of one or more predictive wavenumber variables (i.e., in this study 610.6, 1188.2, 1592.9, 1624.3, 1648.6 and 3288.7 cm⁻¹). In one aspect, the pattern recognition model may detect higher absorbance of 1624.3 and 1188.2 cm⁻¹ and lower absorbance at 610.6, 1592.9, 1648.6 and 3288.7 cm⁻¹ in an FRI patient than a non-FRI patient.

The model may incorporate the spectroscopic profile of a patient along with the biomarker analysis to increase specificity for detecting FRI.

In accordance with one embodiment a method of treating an FRI in a subject is provided wherein the method comprises:
determining if a subject's plasma sample exhibits a biomarker associated with FRI, to identifying a subject having an FRI, wherein the biomarker is
   i) a spectroscopic profile of a plasma sample associated with FRI or
   ii) an increased relative expression of one or more proteins selected from the group consisting of interleukin 6 (IL-6), platelet-derived growth factor AB BB (PDGF-AB BB), and vascular endothelial growth factor A (VEGF-A); and
      administering to said subject identified as having an FRI an anti-infection therapy, optionally wherein the anti-infection therapy is the administration of antibiotics.

In accordance with one embodiment a method of detecting an FRI is provided wherein a plasma sample is subjected to spectrometer analysis to generate a spectroscopic profile. The resulting profile is compared to a reference spectroscopic profile generated from a plasma sample from a healthy subject, and differences in the spectral peaks are assessed to determine the presence of peaks associated with an FRI. In one embodiment the detection of an FRI is associated with elevated expression levels of one or more proteins selected from the group consisting of the group consisting of C-reactive protein (CRP), interleukin 6 (IL-6), platelet-derived growth factor AB BB (PDGF-AB BB), and vascular endothelial growth factor A (VEGF-A).

In one embodiment a method of detecting an FRI in a subject is provided where the method comprises:
obtaining a test spectroscopic profile of a plasma sample obtained from the subject, and
comparing the test spectroscopic profile to a control spectroscopic profile of a plasma sample, wherein higher absorbance at about 1624-1625 cm⁻¹ and/or about 1188-1189 cm⁻¹ and lower absorbance at about 610-611 cm⁻¹, 1592-1593 cm⁻¹, 1648-1649 cm⁻¹, and/or 3288-3289 cm⁻¹ of the test spectroscopic profile when compared to the control spectroscopic profile indicates that the subject likely has an FRI, whereupon an FRI in the subject is detected.

In one embodiment a method of treating an FRI in a subject having FRI is provided, said method comprising:
identifying a subject having an FRI, as a subject having elevated levels of two, three or four proteins selected from the group consisting of C-reactive protein (CRP), interleukin 6 (IL-6), platelet-derived growth factor AB BB (PDGF-AB BB), and vascular endothelial growth factor A (VEGF-A), wherein the identification is determined by
   i) obtaining a reference expression level of proteins selected from the group consisting of C-reactive protein (CRP), interleukin 6 (IL-6), platelet-derived growth factor AB BB (PDGF-AB BB), and vascular endothelial growth factor A (VEGF-A);
   ii) determining an expression level of two, three or four proteins in a sample obtained from a test subject, wherein the two, three or four proteins correspond to those detected in step i), and identifying a subject having FRI as a test subject having an increase in the expression level of said three or more proteins in said patient's sample as compared to the reference expression level; and
administering to said patient having an FRI an anti-infection therapy, optionally wherein the anti-infection therapy is the administration of antibiotics. The method of detecting the expression level of three or more proteins in a sample obtained from a test subject, can be by any detection/quantification technique known to the skilled practitioner including for example using ELISA assays, spectroscopy, or mass spectrometry. In one embodiment the step of identifying a subject having an FRI comprises identification of a subject having elevated levels of all four proteins selected from the group consisting of C-reactive protein (CRP), interleukin 6 (IL-6), platelet-derived growth factor AB BB (PDGF-AB BB), and vascular endothelial growth factor A (VEGF-A),

In one embodiment a method of detecting and treating a fracture-related infection (FRI) in a subject is provided, wherein the method comprises:
obtaining a test spectroscopic profile of a plasma sample obtained from the subject, and
comparing the test spectroscopic profile to a control spectroscopic profile of a plasma sample, wherein higher absorbance at about 1624-1625 cm⁻¹ and/or about 1188-1189 cm⁻¹ and/or lower absorbance at about 610-611 cm⁻¹, 1592-1593 cm⁻¹, 1648-1649 cm⁻¹, and/or 3288-3289 cm⁻¹ of the test spectroscopic profile when compared to the control spectroscopic profile indicates that the subject likely has an FRI, and treating the subject for FRI, optionally with an antimicrobial agent. In one embodiment the serum sample is subjected to analysis by an FTIR spectrometer. In one embodiment the subject is determined to have an FRI, if the higher absorbance is at 1624.3 cm⁻¹ and/or 1188.2 cm⁻¹ and/or the lower absorbance is at 610.6 cm⁻¹, 1592.9 cm⁻¹, 1648.6 cm⁻¹, and/or 3288.7 cm⁻¹ or if the higher absorbance is at 1624.3 cm⁻¹ and 1188.2 cm⁻¹ and the lower absorbance is at 610.6 cm-1, 1592.9 cm-1, 1648.6 cm⁻¹, and 3288.7 cm⁻¹.

In one embodiment the method of determining if a subject has an FRI comprises: obtaining a test protein biomarker profile of a plasma sample obtained from the subject, and comparing the test protein biomarker profile to a control protein biomarker profile of a plasma sample from a healthy subject, wherein C-reactive protein (CRP) below about 2-3 mg/dL, interleukin 6 (IL-6) below about 7-8 pg/mL, platelet-derived growth factor-AB BB (PDGF-AB BB) below about 10,442-10,444 pg/mL, and/or vascular endothelial growth factor A (VEGF-A) below about 77-78 pg/mL indicates that the subject unlikely has an FRI. In one embodiment the protein biomarker profile is obtained using a detectably labeled antibody for CRP, a detectably labeled antibody for IL-6, a detectably labeled antibody for PDGF-AB BB, and/or a detectably labeled antibody for VEGF-A. In one embodiment the subject is determined unlikely to have an FRI, if the detected CRP is below 2.8 mg/dL, IL-6 is below 7.8 pg/mL, PDGF-AB BB is below 10,443 pg/mL, and/or VEGF-A is below 77.5 pg/mL.

In one embodiment the subject is determined to have an FRI, if at least one of CRP, IL-6, PDGF-AB BB, and VEGF-A is above the indicated level, with the sensitivity being at least about 84 and specificity being at least about 69, or when at least two of CRP, IL-6, PDGF-AB BB, and VEGF-A are above the indicated levels, with the sensitivity being at least about 61 and specificity being at least about 76, or when at least three of CRP, IL-6, PDGF-AB BB, and VEGF-A are above the indicated levels, with the sensitivity being at least about 38 and specificity being at least about 92, or when all four of CRP, IL-6, PDGF-AB BB, and VEGF-A are above the indicated levels, with the sensitivity being at least about 23 and specificity being about 100. In one embodiment monokine induced by gamma interferon (MIG) are also measured, wherein elevated levels of MIGs relative to a control sample from a healthy patient indicates that the subject likely has an FRI.

In accordance with one embodiment the methods disclosed herein for detecting FRI in subjects can include one or more parameters selected from fracture region, number of fractures, gender, age, and underlying systemic inflammation diseases.

In accordance with embodiment 1, a method of measuring a spectroscopic profile of a subject's biological sample is provided wherein the method comprises obtaining a test spectroscopic profile of a biological sample obtained from a subject wherein absorbance is measured at any one of
i) about 1624-1625 cm⁻¹;
ii) about 1188-1189 cm⁻¹;
iii) about 610-611 cm⁻¹;
iv) about 1592-1593 cm⁻¹;
v) about 1648-1649 cm⁻¹; or
vi) about 3288-3289 cm⁻¹, or any combination thereof, optionally wherein the biological sample is a urine or blood component, optionally wherein the blood component is selected from plasma or serum.

In accordance with embodiment 2, a method of detecting a patient's biological sample that exhibits two or more of the following:
ii) i) a C-reactive protein (CRP) plasma concentration above about 2 mg/dL;
ii) an interleukin 6 (IL-6) plasma concentration above about 7 pg/mL;
iii) a platelet-derived growth factor-AB BB (PDGF-AB BB) plasma concentration above about 10,442 pg/mL; or
iv) a vascular endothelial growth factor A (VEGF-A) plasma concentration above about 77 pg/mL is provided wherein said method comprises
obtaining a plasma sample from a human patient; and
detecting CRP, IL6, PDGF-AB BB and VEGF-A present in the plasma sample, by contacting the plasma sample with antibodies specific for the corresponding CRP, IL6, PDGF-AB BB and VEGF-A, and detecting binding between said antibodies and their target protein, optionally wherein the biological sample is a urine or blood component, optionally wherein the blood component is selected from plasma or serum.

In accordance with embodiment 3 a method of diagnosing a patient with an FRI is provided where the method comprises determining if a subject's biological sample exhibits a biomarker associated with FRI, wherein the biomarker is
i) a spectroscopic profile of a plasma sample associated with FRI or
ii) an increased relative expression of one or more proteins selected from the group consisting of interleukin 6 (IL-6), platelet-derived growth factor AB BB (PDGF-AB BB), and vascular endothelial growth factor A (VEGF-A), optionally wherein the biological sample is a urine or blood component, optionally wherein the blood component is selected from plasma or serum.

In accordance with embodiment 4 a method of any one of embodiments 1-3 is provided wherein the biological sample is a plasma sample, and the steps of identifying a spectroscopic profile of a plasma sample associated with FRI comprises:
obtaining a test spectroscopic profile of a plasma sample obtained from the subject; and comparing the test spectroscopic profile to a control spectroscopic profile of a plasma sample,
wherein higher absorbance at about 1624-1625 cm⁻¹ and/or about 1188-1189 cm⁻¹ and/or lower absorbance at about 610-611 cm⁻¹, 1592-1593 cm⁻¹, 1648-1649 cm⁻¹, and/or 3288-3289 cm⁻¹ of the test spectroscopic profile when compared to the control spectroscopic profile indicates that the subject has an FRI.

In accordance with embodiment 5 a method of any one of embodiments 1-4 is provided wherein the steps of identifying elevated levels of one or more proteins selected from the group consisting of interleukin 6 (IL-6), platelet-derived growth factor AB BB (PDGF-AB BB), and vascular endothelial growth factor A (VEGF-A) comprises:
obtaining a test protein biomarker profile of a plasma sample obtained from said test subject; and comparing the test protein biomarker profile to a control protein biomarker profile of a plasma sample,
wherein elevated expression of at least one of the proteins in the test protein biomarker profile relative to the control protein biomarker profile identifies a patient having an FRI.

In accordance with embodiment 6 a method of any one of embodiments 1-5 is provided wherein the steps of determining if the subject has elevated levels of said three or more proteins comprises detecting at least three of the following:
i) a C-reactive protein (CRP) plasma concentration above about 2 mg/dL;
ii) an interleukin 6 (IL-6) plasma concentration above about 7 pg/mL;
iii) a platelet-derived growth factor-AB BB (PDGF-AB BB) plasma concentration above about 10,442 pg/mL; or
iv) a vascular endothelial growth factor A (VEGF-A) plasma concentration above about 77 pg/mL in a plasma sample form said subject.

In accordance with embodiment 7 a method of any one of embodiments 1-6 is provided wherein said proteins are quantified using an antibody that specifically binds to the respective proteins.

In accordance with embodiment 8 a method of treating a fracture-related infection (FRI) in a subject having FRI is provided wherein the method comprises:
analyzing a subject's plasma sample to detect a biomarker associated with FRI, and identifying a subject having an FRI, wherein the biomarker is
   i) a spectroscopic profile of a plasma sample associated with FRI or
   ii) an increased relative expression of one or more proteins selected from a test profile comprising interleukin 6 (IL-6), platelet-derived growth factor AB BB (PDGF-AB BB), and vascular endothelial growth factor A (VEGF-A); and
administering to said subject identified as having an FRI an anti-infection therapy, optionally wherein the anti-infection therapy is the administration of antibiotics.

In accordance with embodiment 9 a method of any one of embodiments 1-8 is provided wherein the steps of identifying a spectroscopic profile of a plasma sample associated with FRI comprises:
obtaining a test spectroscopic profile of a plasma sample obtained from the subject; and comparing the test spectroscopic profile to a control spectroscopic profile of a plasma sample,
wherein higher absorbance at
   i) about 1624-1625 cm⁻¹;
   ii) about 1188-1189 cm⁻¹; or
   iii) a combination of i) and ii) and/or
lower absorbance at
   iv) about 610-611 cm⁻¹;
   v) about 1592-1593 cm⁻¹;
   vi) about 1648-1649 cm⁻¹;
   vii) about 3288-3289 cm⁻¹ or
   viii) or any combination of iv-vii
      of the test spectroscopic profile when compared to the control spectroscopic profile indicates that the subject has an FRI.

In accordance with embodiment 10 a method of any one of embodiments 1-9 is provided wherein the higher absorbance is at
i) about 1624.3 cm⁻¹;
ii) about 1188.2 cm⁻¹; or
iii) a combination of i) and ii) and/or
the lower absorbance is at
iv) about 610.6 cm⁻¹;
v) about 1592.9 cm⁻¹;
vi) about 1648.6 cm⁻¹;
vii) about 3288.7 cm⁻¹ or
viii) or any combination of iv-vii.

In accordance with embodiment 11 a method of any one of embodiments 1-10 is provided wherein the higher absorbance is at
about 1624.3 cm⁻¹; and about 1188.2 cm⁻¹; and the lower absorbance is at 610.6 cm-1, 1592.9 cm⁻¹, 1648.6 cm⁻¹, and 3288.7 cm⁻¹.

In accordance with embodiment 12 a method of any one of embodiments 1-11 is provided wherein the spectrometer is an FTIR spectrometer.

In accordance with embodiment 13 a method of any one of embodiments 1-12 is provided wherein the steps of identifying elevated levels of one or more proteins selected from the test profile comprises:
determining the expression profile of one or more proteins selected from said test profile in a plasma sample obtained from said test subject relative to the corresponding expression profile of one or more proteins selected from said test profile in a plasma sample obtained from a control plasma sample,
wherein elevated expression of at least one of the proteins in the test protein biomarker profile in the test subject's plasma sample relative to the control plasma sample identifies a patient having an FRI.

In accordance with embodiment 14 a method of any one of embodiments 1-13 is provided wherein the plasma sample is analyzed for elevated expression of two or more proteins selected from the group consisting of C-reactive protein (CRP), IL-6, PDGF-AB BB and VEGF-A.

In accordance with embodiment 15 a method of any one of embodiments 1-14 is provided wherein the plasma sample is analyzed for:
i) a C-reactive protein (CRP) plasma concentration above about 2 mg/dL;
ii) an interleukin 6 (IL-6) plasma concentration above about 7 pg/mL;
iii) a platelet-derived growth factor-AB BB (PDGF-AB BB) plasma concentration above about 10,442 pg/mL; or
iv) a vascular endothelial growth factor A (VEGF-A) plasma concentration above about 77 pg/mL wherein detection of any two of i), ii), iii) and iv) identifies a subject having an FRI.

In accordance with embodiment 16 a method of any one of embodiments 1-15 is provided wherein the plasma sample is analyzed for:
i) a C-reactive protein (CRP) plasma concentration above about 2.8 mg/dL;
ii) an interleukin 6 (IL-6) plasma concentration above about 7.8 pg/mL;
iii) a platelet-derived growth factor-AB BB (PDGF-AB BB) plasma concentration above about 10,443 pg/mL; or
iv) a vascular endothelial growth factor A (VEGF-A) plasma concentration above about 77.5 pg/mL wherein detection of any two of i), ii), iii) and iv) identifies as a subject having an FRI.

In accordance with embodiment 17 a method of any one of embodiments 1-16 is provided wherein said proteins are quantified using an antibody that specifically binds to the respective proteins.

In accordance with embodiment 18 a method of any one of embodiments 15-17 is provided wherein three of i) through iv) are detected.

In accordance with embodiment 19 a method of any one of embodiments 1-11 is provided wherein the detection of CRP above 2.8 mg/dL, IL-6 above 7.8 pg/mL, PDGF-AB BB above 10,443 pg/mL, and VEGF-A above 77.5 pg/mL identifies as a subject having an FRI.

In accordance with embodiment 20 a method of any one of embodiments 1-19 is provided, further comprising the step of measuring monokine induced by gamma interferon (MIG) in a subjects plasma, wherein detected elevated levels of MIG relative to a control sample indicates a subject having an FRI.

In accordance with embodiment 21 a method of treating a fracture-related infection (FRI) in a subject having FRI, said method comprising:
identifying a subject having elevated levels of three or more proteins selected from the group consisting of C-reactive protein (CRP), interleukin 6 (IL-6), platelet-derived growth factor AB BB (PDGF-AB BB), and vascular endothelial growth factor A (VEGF-A), as a subject having an FRI; and
administering to said subject identified having an FRI, an anti-infection therapy.

### ALTERNATIVE EMBODIMENTS

1. A method of treating a fracture-related infection (FRI) in a subject having FRI, said method comprising:
analyzing a subject's biological sample using an analytical method to determine whether a subject has an FRI; and
administering an anti-infection therapy to said subject identified as having an FRI.

2. The method of embodiment 1, wherein the analytical method provides an analytical patient profile.

3. The method of embodiment 1 or 2, wherein the anti-infection therapy comprises an anti-infective.

4. The method of embodiment 3, wherein the anti-infective comprises a cephalosporin, linezolid, dalbavancin, metronidazole, a fluoroquinoline, gentamicin, tobramycin, vancomycin, clindamycin, an antifungal, or any combination thereof.

5. The method of any one of the preceding embodiments, wherein the biological sample comprises blood, urine, a synovial fluid, or a combination thereof.

6. The method of any one of the preceding embodiments, wherein the biological sample comprises blood plasma.

7. The method of any one of the preceding embodiments, wherein the biological sample comprises a biomarker.

8. The method of embodiment 7, wherein the biomarker comprises a protein, a non-protein metabolite, a xenobiotic, heme, a nucleic acid, or any combination thereof.

9. The method of embodiment 8, wherein the biomarker comprises a lipid, a sugar, a beta hydroxy acid, an amino acid, a bile acid, or a derivative thereof.

10. The method of embodiment 9, wherein the biomarker comprises myristoleate, 14-methylpalmitate, 15-methylpalmitate, 16-hydroxypalmitate, 10-undecenoate, palmitoleate, 12-methylmyristate, 13-methylmyristate, 5-dodecenoate, pentadecanoate, myristate, glycholate, hexadecadienoate, eicosapentaenoate, hexadecanedioate, 1-linoleoyl-2-arachidonoyl-GPC (18:2/20:4n6), 1-palmitoyl-2-arachidonoyl-GPI, -linolenoyl-GPC (18:3), or any combination thereof.

11. The method of embodiment 9, wherein the amino acid or a derivative thereof comprises alanine, N-lactoyl-isoleucine, N-lactoyl-tyrosine, N-acetyl-glutamide, methionine sulfoxide, or any combination thereof.

12. The method of embodiment 8, wherein the biomarker comprises benzoate, vanilloylglycine, 3-hydroxy-2-methylpyridine sulfate, hippurate, 2-hydroxyhippurate (salicylurate), levulinate (4-oxovalerate), ferulic acid 4-sulfate, vanillic alcohol, a derivative thereof, or any combination thereof.

13. The method of embodiment 9, wherein the beta hydroxy acid comprises 3-hydroxybutyrate (BHBA).

14. The method of embodiment 9, wherein the bile acid comprises glycocholate, glycochenodeoxycholate, taurochenodeoxycholate, or any combination thereof.

15. The method of embodiment 8, wherein the nucleic acid comprises deoxyribonucleic acid (DNA), ribonucleic acid (RNA), or a combination thereof.

16. The method of embodiment 15, wherein the RNA comprises microRNA.

17. The method of embodiment 8, wherein the biomarker comprises alpha-1-acid glycoprotein 1, serum amyloid A-2 protein, leucine-rich alpha-2-glycoprotein, complement factor H-related protein 5, C-reactive protein (CRP), complement component C9, haptoglobin, serum amyloid A-1 protein, lipopolysaccharide-binding protein, peptidyl-prolyl cis-trans isomerase A, calmodulin-3, fructose-bisphosphate aldolase A, complement factor B, peroxiredoxin-2, mannosyl-oligosaccharide 1,2-alpha-mannosidase IA, alpha-1-acid glycoprotein 2, Transgelin-2, complement C1r subcomponent, retinoic acid receptor responder protein 2, ceruloplasmin, complement C1s subcomponent, carboxypeptidase N catalytic chain, alpha-2-macroglobulin, coagulation factor X, Insulin-like growth factor-binding protein 4, selenoprotein P (SEPP1), fibronectin, cell surface glycoprotein MUC18, hepatocyte growth factor activator, apolipoprotein A-IV, plasma kallikrein, afamin, platelet-derived growth factor AB/BB (PDGF-AB/BB), monokine induced by gamma interferon (MIG), Interleukin 6 (IL-6), vascular endothelial growth factor A (VEGF-A), fibrin, procalcitonin, neutrophil CD64 (nCD64), CD66b, or any combination thereof.

18. The method of embodiment 17, wherein the biomarker comprises CRP, PDGF-AB/BB, MIG, IL-6, or VEGF-A.

19. The method of any one of the preceding embodiments, wherein the step of analyzing a subject's biological sample comprises:
obtaining an analytical patient profile of a biological sample obtained from the subject; and
comparing the analytical patient profile to a reference analytical patient profile.

20. The method of embodiment 19, wherein the step of obtaining an analytical patient profile further comprises a step of diluting a biological sample with potassium thiocyanate.

21. The method of embodiment 19 or 20, wherein the step of comparing the analytical patient profile to a reference analytical patient profile comprises determining if the biological sample comprises an increased amount or a decreased amount of a biomarker compared to a reference analytical patient profile.

22. The method of embodiment 21, wherein the biological sample comprises an increased amount of a biomarker compared to a reference analytical patient profile.

23. The method of embodiment 21, wherein the biological sample comprises a decreased amount of a biomarker compared to a reference analytical patient profile.

24. The method of any one of the preceding embodiments, wherein the analytical method comprises a mass spectrometer.

25. The method of any one of the preceding embodiments, wherein the analytical method comprises a Fourier-transform infrared spectroscopy (FTIR) spectrometer.

26. The method of any one of embodiment 24, wherein the analytical method comprises Tandem mass tag liquid chromatography-mass spectrometry (TMT LC-MS/MS), a reverse phase ultrahigh performance liquid chromatography-tandem mass spectroscopy (RP)/UPLC-MS/MS method using positive ion mode electrospray ionization (ESI), or a (RP)/UPLC-MS/MS method using negative ion mode ESI.

27. The method of any one of the preceding embodiments, wherein the subject is a human.

28. The method of any one of the preceding embodiments, wherein the subject is an adult.

29. The method of any one of the preceding embodiments, wherein the FRI is associated with a surgical implant; or the introduction of an implant, the replacement of an implant, or the adjustment of an implant.

30. The method of any one of the preceding embodiments, wherein the FRI is caused by one or more pathogens.

31. The method of any one of the preceding embodiments, wherein the FRI is caused by more than one pathogen.

32. The method of embodiment 30 or embodiment 31, wherein the pathogen comprises a Gram-positive bacterium, a Gram-negative bacterium, an anaerobic bacterium, a mycobacterium, a fungus, or any combination thereof.

33. The method of embodiment 32, wherein the pathogen comprises a Gram-positive bacterium.

34. The method of embodiment 33, wherein the Gram-positive bacterium comprises one or more Staphylococcus species.

35. The method of embodiment 34, wherein the Gram-positive bacterium comprises Staphylococcus aureus, Staphylococcus epidermidis, or a combination thereof.

36. The method of embodiment 32, wherein the pathogen comprises a Gram-negative bacterium.

37. The method of embodiment 36, wherein the Gram-negative bacterium comprises Pseudomonas aeruginosa, Escherichia coli, or a combination thereof.

### EXAMPLES

The following examples serve to illustrate the present disclosure. The examples are not intended to limit the scope of the claimed invention.

### Example 1. Four Biomarkers Accurately Differentiated FRI Patients from Matched Control Patients

### 1A. Patient Selection Methodology

The study was performed over a period of nine months. Inclusion criteria for both the confirmed FRI and control groups were age 18 to 85 years and an extremity, pelvic ring, or acetabulum fracture that was surgically treated with retained orthopaedic implant within the last two years. Additionally, specifically for the confirmed FRI group, a clinically suspected FRI was required for inclusion. Exclusion criteria included the following: hand and spine fractures, pregnancy, incarceration, known immunosuppressive state (e.g., lupus, cancer, human immunodeficiency virus (HIV), hepatitis C, rheumatologic disease, or any patient taking an immune-modulating medication), known separate source infection (e.g., urinary tract infection, pneumonia, decubitus ulcer), systemic infection (e.g., sepsis or bacteremia), undergoing second debridement or prior failed infection treatment, pathologic fracture, definitive treatment without retained implant (i.e., arthroplasty, percutaneous Kirschner wires, or external fixation), known venous thromboembolism, and hemodialysis. All FRI confirmed patients were enrolled in the study and blood samples were obtained prior to surgical intervention for treatment of the infected site. Non-infected control patients were identified and matched to the FRI patients based on age (±15 years), time after surgery (±2 weeks), and fracture region. Fracture regions were matched as follows: upper extremity long bones (humerus, radius, and ulna), lower extremity long bones (femur and tibia), and other lower extremity bones (e.g., patella, tarsal bones of the foot). Control patients were identified through routine clinical follow-up. All controls remained infection-free for a minimum of 6 months after enrollment as determined by routine clinic follow-up, chart review, or phone calls.

### 1B. Patient Cohort Data

Eighty-two patients were screened for enrollment. Thirty-two patients were screen failures (alternative source of infection (n=3), pathologic fracture (n=3), thromboembolic disease (n=1), age <18 (n=2), immunosuppressive state (n=5), unable to provide consent (n=1), HIV+(n=1), undergoing second treatment or prior failure of infection treatment (n=13), definitive management including percutaneous Kirshner wires or external fixation pins (n=1), and hemodialysis (n=2)), 8 declined to participate, and 2 were unable to be enrolled due to difficulty collecting blood samples. Ultimately, 40 patients were enrolled in the study and had blood samples collected. Two cases were later withdrawn after enrollment; one due to discovery of prior nonunion surgery and the other due to detection of a separate infectious source (i.e., urinary tract infection). The final cohort included 22 confirmed FRIs and 16 controls. Using the above-described matching criteria, 13 pairs of confirmed FRIs were matched with controls. All 13 of the FRIs met confirmatory criteria with either fistula/sinus/wound breakdown and/or purulent drainage on initial presentation. Eight of 13 (62%) had at least 2 positive cultures with phenotypically indistinguishable pathogens from their infection surgery.

Table 1 summarizes patient demographic, clinical, and co-morbidity data for FRIs and controls. The mean enrollment time was 6 weeks post-operative, with 69% (18/26) of the entire cohort having femur or tibia bone involvement, 54% (14/26) having plate fixation, and 15% (4/26) having an open fracture. As Table 1 shows, there was no significant difference in age or fracture region.

TABLE 1
Description of Cohort*

| | Overall Cohort | | FRI | | Control | | |
|---|---|---|---|---|---|---|---|
| | (n = 26) | | (n = 13) | | (n = 13) | | p-value** |
| Demographics | | | | | | | |
| Age | 51.3 | (14.9) | 51.4 | (14.8) | 51.2 | (15.5) | 0.953 |
| Sex | | | | | | | 0.226 |
| Male | 16 | | 10 | | 6 | | |
| Female | 10 | | 3 | | 7 | | |
| BMI | 30.2 | (7.9) | 28 | (4.6) | 32.4 | (9.9) | 0.209 |
| Weeks Post-Operation | 6.0 | (4.3) | 6.4 | (4.5) | 5.5 | (4.4) | 0.043 |

| | | | | |
|---|---|---|---|---|
| Clinical | | | | |
| Bone Involvement | | | | >0.999 |
| Femur/Tibia | 18 | 9 | 9 | |
| Patella/Ankle/Foot | 6 | 3 | 3 | |
| Upper Extremity/Clavicle | 2 | 1 | 1 | |
| Implant Used | | | | 0.047 |
| IMN | 12 | 9 | 3 | |
| Plate | 14 | 4 | 10 | |
| Fracture Type | | | | 0.096 |
| Open | 4 | 4 | 0 | |
| Closed | 22 | 9 | 13 | |
| NSAID/Steroid Use | | | | >0.999 |
| Yes | 1 | 1 | 0 | |
| No | 25 | 12 | 13 | |
| Co-morbidities | | | | |
| Diabetes Mellitus | | | | 0.322 |
| Yes | 5 | 1 | 4 | |
| No | 21 | 12 | 9 | |
| History of MRSA | | | | 0.48 |
| Yes | 2 | 2 | 0 | |
| No | 24 | 11 | 13 | |
| Tobacco Use | | | | >0.999 |
| Yes | 7 | 4 | 3 | |
| No | 19 | 9 | 10 | |
| Alcohol Abuse | | | | 0.48 |
| Yes | 2 | 2 | 0 | |
| No | 24 | 11 | 13 | |

*Values are means (S.D.) for continuous data (i.e., age, BMI, weeks post-operation). All other values are counts.
**Result from two-sided matched t-test for continuous data and Fisher's Exact test for categorical data.

1C. Collecting and Processing Blood from Patients

ESR, CRP, and WBC, as well as three intra-operative cultures and gram stains, were obtained as part of the standard of care for the FRI patients. Peripheral blood samples were obtained from the FRI cohort on the day of surgery for infection. Specifically, an EDTA purple top tube (BD Vacutainer®, Becton, Dickinson and Company, Franklin Lakes, NJ) was utilized for collection of approximately 5 mL of whole blood. The tube was inverted 4-5 times to allow the blood to mix with the anticoagulant. The tube was placed and balanced in a table-top centrifuge and spun at 1500 g for 10 minutes with acceleration and deceleration set at 9. This default setting allows the centrifuge to reach not only the set centrifugal force (1500 g) but also brake or decelerate in the shortest time following the spin. Plasma was then extracted, aliquoted into 500 microliter tubes, and stored at −80° C. Blood samples for controls were collected from patients during routine post-operative clinic visits with the same collection and processing as described above.

### 1D. Plasma Protein Selection and Measurements

An assay kit was used for protein multiplex ELISA (EMD Millipore Corporation, Burlington, MA). The panel contained the following 48 proteins: soluble CD40L (sCD40L), Epidermal growth factor (EGF), Eotaxin, basic fibroblast growth factor 2 (FGF-2), Fms-related tyrosine kinase 3 ligand (FLT-3L), Fractalkine, Granulocyte colony-stimulating factor 3 (G-CSF), Granulocyte macrophage colony-stimulating factor GM-CSF, C-X-C motif ligand 1 (CXCL1), C-X-C motif chemokine ligand 10 (CXCL10), Interferons selected from: IFNα2, IFNγ, interleukins selected from: IL1α, IL-1β, IL-1RA, IL-2, IL-3, IL-4, IL-5, IL-6, IL-7, IL-8, IL-9, IL-10, IL-12 (p40), IL-12 (p70), IL-13, IL-15, IL-17A, IL-17E/IL-25, IL-17F, IL-18, IL-22, IL-27, Monocyte Chemoattractant Protein-1 (MCP-1), Monocyte Chemoattractant Protein-3 (MCP-3), Macrophage colony-stimulating factor (M-CSF), Macrophage-derived chemokine (MDC), Monokine induced by gamma (MIG), Macrophage inflammatory protein-1 alpha (MIP-la), Macrophage inflammatory protein-1 beta (MIP-1β), Platelet Derived Growth Factor-AA (PDGF-AA), Platelet Derived Growth Factor-AB/BB (PDGF-AB BB), RANTES, transforming growth factor α (TGFα), TNFα, transforming growth factor β (TGFβ), and endothelial growth factor A (VEGF-A). This immunoassay was selected because it contains a substantial number of relevant inflammatory biomarkers that have been associated with the inflammatory response to infection. Additionally, CRP levels were measured on all samples. For samples with biomarker concentrations that were undetectable, (½*lowest detectable value) was used for data analysis. Any samples that exceeded the maximal detectable value were diluted, re-measured, and corrected for dilution.

### 1E. Plasma Sample Analysis

Of the 49 plasma proteins assessed, seven trended (p<0.1) towards being significantly different between the FRI and control groups, with 4 of these having p<0.05. Tables 2 and 3 show the plasma concentrations for all proteins assessed. As IL-6, PDGF-AB BB, VEGF-A, and CRP were significantly different between the two groups, they were carried forward into ROC curve analyses. Cut-points optimizing the ROC analyses were 7.8 pg/mL, 10,443 pg/mL, 77.5 pg/mL for IL-6, PDGF-AB BB, and VEGF-A, respectively. Cut point was 2.8 mg/dL for CRP. Areas under the curve (AUCs) for these cut points ranged from 0.654-0.731 (See Table 4). Examined cumulatively, having all four of these biomarkers below their respective cut-points was 100% specific for FRI (See Table 5).

TABLE 2
Statistically Significant Biomarkers

| Biomarker* | FRI (n = 13) | | Control (n = 13) | | p-value** |
|---|---|---|---|---|---|
| CRP | 5.61 | (6.92) | 1.17 | (1.36) | 0.046 |
| IL-6 | 26.45 | (35.36) | 3.67 | (2.57) | 0.036 |
| PDGF-AB BB | 15561 | (8926) | 7773 | (2656) | 0.023 |
| VEGF-A | 98.88 | (96.56) | 37.36 | (18.33) | 0.033 |

*Values are means (S.D.) for concentration of biomarker in plasma. Units are mg/dL for CRP and pg/mL for all others.
**Result from two-sided matched t-test.

TABLE 3
Other Biomarkers*

| | | | Detectable | | | Detectable | |
|---|---|---|---|---|---|---|---|
| Biomarker | FRI | | (n)** | Control | | (n) | p-value*** |
| MCP-1 | 275.97 | (125.44) | 13 | 275.70 | (106.88) | 13 | 0.996 |
| MIP-1a | 18.52 | (29.93) | 6 | 19.33 | (16.63) | 8 | 0.943 |
| IL-3 | 0.53 | (0.85) | 2 | 0.57 | (1.21) | 2 | 0.932 |
| IL-12(P40) | 70.39 | (52.41) | 13 | 68.37 | (41.01) | 13 | 0.914 |
| IL-17F | 39.89 | (119.44) | 3 | 34.40 | (80.04) | 8 | 0.899 |
| TGFa | 5.99 | (17.58) | 4 | 5.18 | (11.71) | 8 | 0.899 |
| M-CSF | 88.16 | (123.20) | 10 | 97.89 | (112.53) | 8 | 0.856 |
| IL-13 | 26.49 | (37.94) | 9 | 23.54 | (14.79) | 10 | 0.772 |
| IL-10 | 9.28 | (12.16) | 10 | 8.06 | (8.3) | 9 | 0.757 |
| IL-22 | 31.03 | (82.84) | 3 | 23.07 | (27.10) | 5 | 0.718 |
| IL-1a | 13.63 | (33.58) | 9 | 9.27 | (7.23) | 12 | 0.679 |
| FLT-3L | 14.05 | (10.68) | 13 | 15.93 | (6.50) | 13 | 0.651 |
| IL-1RA | 8.29 | (8.19) | 13 | 10.18 | (8.99) | 13 | 0.607 |
| IFNa2 | 44.99 | (52.47) | 11 | 33.98 | (28.92) | 12 | 0.571 |
| MCP-3 | 22.97 | (38.66) | 10 | 16.55 | (7.79) | 13 | 0.553 |
| IL-1b | 12.74 | (36.93) | 7 | 6.05 | (6.61) | 10 | 0.551 |
| IL-8 | 4.24 | (2.37) | 13 | 3.80 | (1.94) | 13 | 0.528 |
| TNFa | 28.06 | (17.30) | 13 | 24.61 | (7.66) | 13 | 0.526 |
| IL-2 | 1.11 93.04) | | 4 | 0.50 | (0.71) | 7 | 0.518 |
| IL-17E IL-25 | 251.70 | (688.11) | 5 | 388.53 | (265.77) | 12 | 0.504 |
| IL-4 | 2.40 | (4.79) | 8 | 1.44 | (1.96) | 10 | 0.500 |
| IL-17A | 7.87 | (21.83) | 7 | 2.78 | (2.62) | 9 | 0.436 |
| FGF-2 | 98.09 | (195.80) | 13 | 53.53 | (17.96) | 13 | 0.434 |
| Fractalkine | 151.08 | (188.64) | 12 | 103.26 | (60.92) | 12 | 0.390 |
| GM-CSF | 3.19 | (2.95) | 1 | 2.42 | (0.84) | 1 | 0.387 |
| IP-10 | 231.20 | (204.91) | 13 | 173.65 | (69.33) | 13 | 0.376 |
| IL-18 | 30.00 | (33.84) | 12 | 40.44 | (21.69) | 13 | 0.376 |
| G-CSF | 66.81 | (73.73) | 11 | 47.63 | (38.92) | 11 | 0.368 |
| IL-5 | 5.08 | (4.19) | 13 | 7.22 | (5.50) | 13 | 0.345 |
| TNFb | 3.66 | (9.53) | 7 | 1.08 | (1.74) | 7 | 0.327 |
| MIP-1b | 32.42 | (48.67) | 13 | 17.94 | (5.79) | 13 | 0.314 |
| IL-12(P70) | 9.89 | (30.34) | 7 | 1.28 | (2.00) | 5 | 0.312 |
| Eotaxin | 53.25 | (34.96) | 13 | 70.38 | (39.74) | 13 | 0.281 |
| IFNr | 7.70 | (19.63) | 9 | 1.43 | (1.18) | 12 | 0.268 |
| Rantes | 94985 | (58356) | 13 | 74073 | (28388) | 13 | 0.264 |
| IL-15 | 7.50 | (4.59) | 13 | 12.57 | (12.85) | 13 | 0.263 |
| IL-9 | 9.76 | (12.82) | 10 | 17.50 | (15.83) | 12 | 0.262 |
| IL-7 | 1.76 | (2.10) | 9 | 1.07 | (1.35) | 8 | 0.239 |
| GROa | 16.88 | (16.19) | 9 | 10.50 | (8.03) | 9 | 0.230 |
| MIG CXCL9 | 2673.6 | (2636.5) | 13 | 1535.8 | (665.41) | 13 | 0.184 |
| IL-27 | 2775.4 | (1938.1) | 13 | 1840.5 | (983.3) | 13 | 0.149 |
| EGF | 47.68 | (61.93) | 11 | 16.90 | (10.01) | 12 | 0.117 |
| sCD40L | 512.78 | (472.19) | 13 | 242.57 | (121.53) | 13 | 0.078 |
| MDC | 754.8 | (196.09) | 13 | 1057.4 | (534.13) | 13 | 0.075 |
| PDGF-AA | 1278.1 | (962.86) | 13 | 633.15 | (342.35) | 13 | 0.068 |

*Values are means (S.D.) for concentration of biomarker in plasma. Units are in pg/mL.
**Not all patients had detectable levels of each biomarker. The number in this column shows how many patients had a detectable level.
***Result from two-sided matched t-test.

TABLE 4
ROC Optimization

| Biomarker | Cut-Point | | Sensitivity | Specificity | AUC* |
|---|---|---|---|---|---|
| CRP | 2.8 | mg/dL | 46.2 | 92.3 | 0.692 |
| IL-6 | 7.8 | pg/mL | 53.9 | 84.6 | 0.692 |
| PDGF-AB BB | 10,443 | pg/mL | 61.5 | 84.6 | 0.731 |
| VEGF-A | 77.5 | pg/mL | 38.5 | 92.3 | 0.654 |

*AUC = Area under the curve. Logistic regression models were performed to determine the AUC, as well as to derive the cut-points for the Youden index.

TABLE 5
Cumulative Diagnostic Parameters for
CRP, IL-6, PDGF-AB BB, and VEGF-A

| Markers above cut-point | Sensitivity | Specificity | AUC* |
|---|---|---|---|
| At least 1 of the 4 | 84.6 | 69.2 | .769 |
| At least 2 of the 4 | 61.5 | 76.9 | .692 |
| At least 3 of the 4 | 38.5 | 92.3 | .654 |
| At least 4 of the 4 | 23.1 | 100 | .615 |

*AUC = Area under the curve. Logistic regression models were performed to determine the AUC, as well as to derive the cut-points for the Youden index.

### 1F. Statistical Analysis

Plasma protein differences between the FRI and control groups were assessed using two-sided matched t-tests. Although change/paired data are typically linear, plasma protein results can be skewed, so non-parametric signed rank tests were also performed to verify the results of the paired t-tests (similar findings, results not shown).

To analyze the predictability of plasma proteins to categorize treatment group participants, logistic regression models were performed, and ROC curves were generated to determine the optimal cut-points for each protein, using the Youden J Index for this optimal cut-point. A cumulative index, ranging from 0 to 4, for the four significant (p<0.05) proteins was also calculated by summing the number of proteins that were above separate Youden values, and ROC analyses were performed on each category (having at least one, having at least two, etc.) in order to determine if this can give a more accurate prediction. All analytic assumptions were verified, and all analyses were performed using SAS v9.4 (SAS Institute, Cary, NC).

For multivariate analysis, both data sets (protein measurements and MIR spectra) were imported into the MATLAB® software (MathWorks R2015b (8.6.0.267246), Natick, MA). In-house written scripts were utilized for processing. Initially, spectral data were smoothed using the Savitsky-Golay filter (2^{nd} degree polynomial functions and 11-point smoothing window). Standard normal variate transform (SNV) and baseline normalization to the KSCN peak were used for spectral normalization. Verification of whether an observation was an outlier or not relied on the values of the two statistics, T² and Q, for both of which the null hypothesis was tested at a 95% confidence level. The average of the three replicates for each sample was used for analysis. Statistical significance was set at P<0.05.

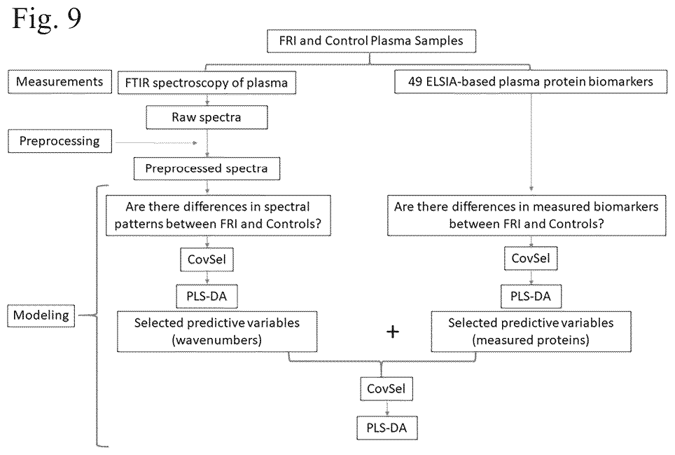

To allow comparison of the utility of ELISA-based proteins and sample spectral patterns as predictors of FRI, both data sets were used to build multivariate classification models to discriminate between FRI and control samples, with subsequent validation. Partial least squares discriminant analysis (PLS-DA) was used for classification to address the low number of patient samples in the training set compared to the number of measured variables for both data sets. In order to improve classification accuracy and to identify a minimum set of proteins and spectral wavenumbers out of the whole set of variables, the PLS-DA classification algorithm was coupled with covariance selection (CovSel). The CovSel-PLS-DA model was built and validated through a repeated double-cross-validation (rDCV) procedure with 13 segments in the outer loop and 12 in the inner loop using 50 repetitions. For each cancelation group in the outer loop, predictions were carried out on a model built on the remaining samples. The best subset of original variables to be used as inputs and the optimal number of latent variables were selected as those leading to the minimum classification error in the inner-loop cross-validation procedure. Data were auto-scaled prior to the analysis. Lastly, the selected variables from the two platforms were integrated in a mid-level data fusion approach. The predictors were auto-scaled, and the proteins and MIR spectra data were further block-scaled to allow equal contributions. For each comparison the accuracy, sensitivity, and specificity of the predictive model, as well as the AUC of the ROC curve, are reported as measures of the model's performance. Exemplary steps of multivariate analysis are summarized in FIG. 9.

### Example 2. Quick Spectroscopy Analysis of Plasma has a Similar Sensitivity and Accuracy as an ELISA

ELISA is the gold standard for identifying the presence and relative expression levels of biomolecules. However, ELISA is expensive and is not useful as a quick diagnostic method. The following exemplifies that the disclosed method is similarly sensitive and accurate as the antibody test with the added advantage of being substantially cheaper and providing results faster.

### 2A. Infrared Spectroscopy

Thawed plasma samples were diluted with potassium thiocyanate (KSCN, SigmaUltra, Sigma-Aldrich Inc, St Louis, MO) as an internal control in a 2:1 ratio. Using a previously described technique, three 8 μL replicates of each sample were applied on a 96-welled silicon microplate and allowed to dry at room temperature (20-22° C.). Each microplate was placed in the multi-sampler (HTS-XT, Bruker Scientific, LLC, Billerica, MA, USA) attachment of an FTIR spectrometer (INVENIO S, Bruker Scientific, LLC, Billerica, MA, USA). Mid-IR (MIR) absorbance spectra in the wavenumber range of 400 to 4,000 cm⁻¹ was recorded using the OPUS software (version 6.5, Bruker Optics, GmbH, Ettlingen, Germany). For each sample evaluation, 512 interferograms were signal averaged and Fourier-transformed to produce a nominal resolution of 4 cm⁻¹ for the resulting spectrum.

### 2B. Multivariate Analysis of Biomarkers

A multivariate analysis-based predictive model developed utilizing ELISA-based biomarkers had sensitivity, specificity, and accuracy of 69.2±0.0%, 99.9±1.0%, and 84.5±0.6%, respectively, with PDGF-AB/BB, CRP, and MIG (i.e., CXCL9) selected as the minimum number of variables explaining group differences. Sensitivity, specificity, and accuracy of the predictive model based on MIR spectra were 69.9±6.2%, 71.9±5.9%, and 70.9±4.8%, respectively, with six wavenumbers as explanatory variables (3288.7, 1648.6, 1624.3, 1592.9, 1188.2 and 610.6 cm⁻¹).

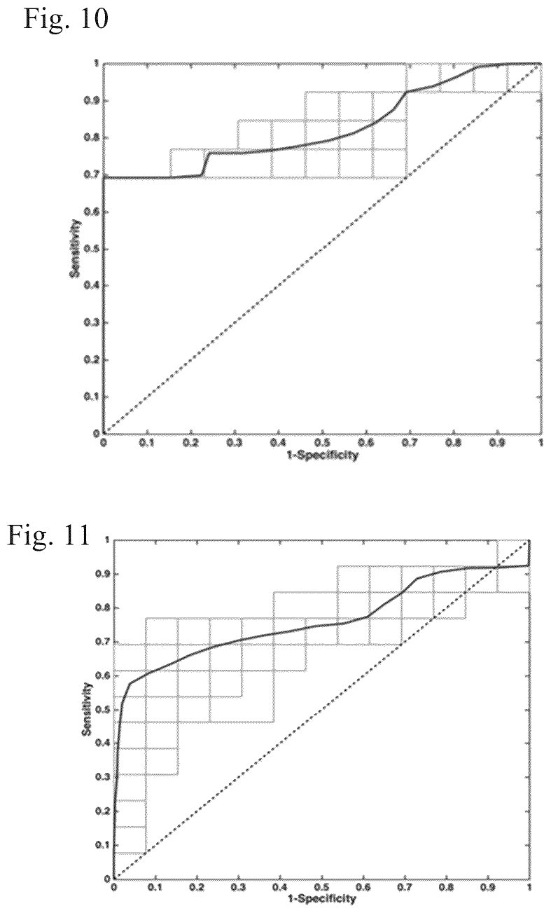

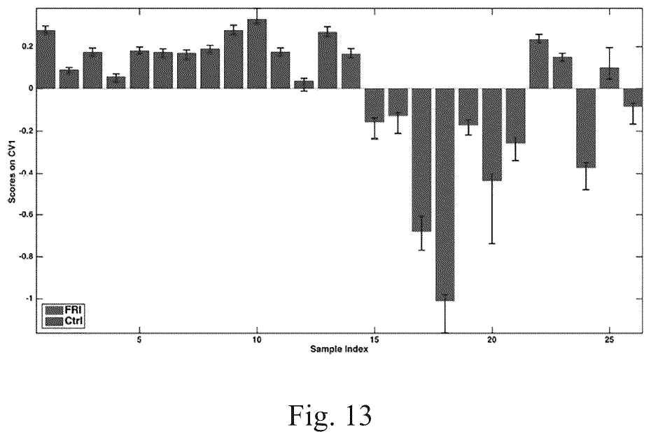

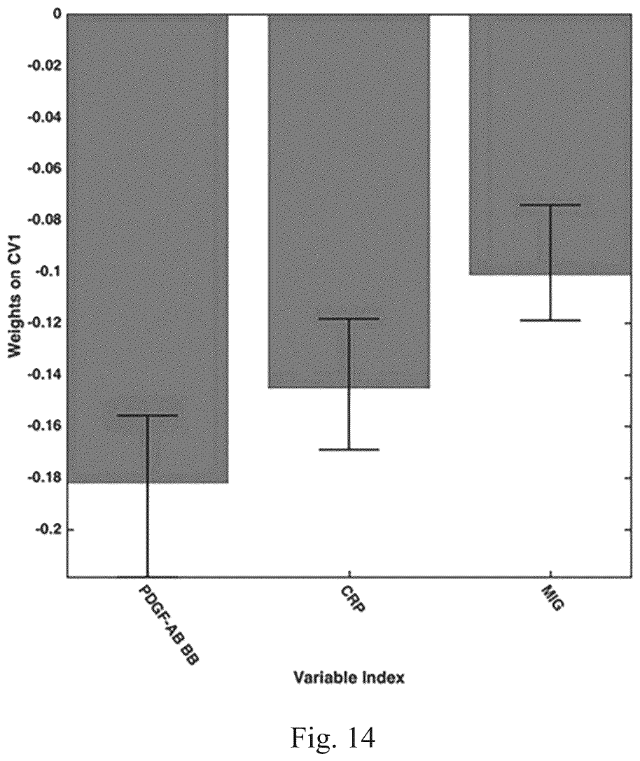

For multivariate analysis using plasma protein levels as predictors resulted in PDGF-AB/BB, CRP, and MIG being retained to build the classification model. The overall classification accuracy on the external loop samples was found to be 84.5±0.6%, with 69.2±0.0% sensitivity and 99.9±1.0% specificity (FIG. 10). The AUC for this model was 0.826±0.018, demonstrating an excellent discriminant ability. The visual interpretation of the results based on comparing the scores of the outer loop samples along the only canonical variate of the model with the variable weights for the construction of that direction is presented in FIG. 13 and FIG. 14.

### 2C. FTIR Spectroscopy

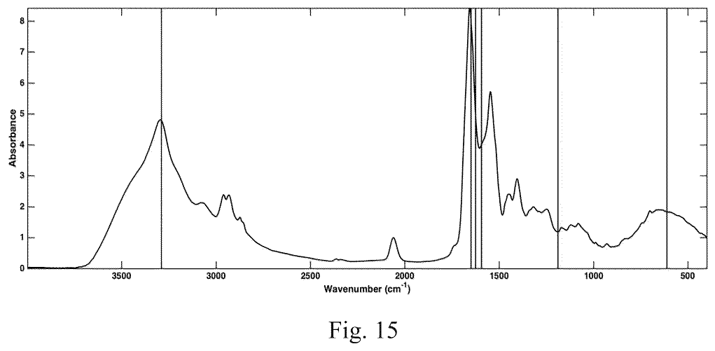

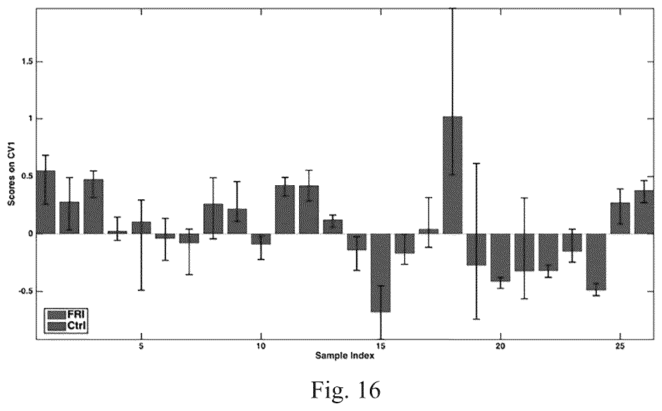

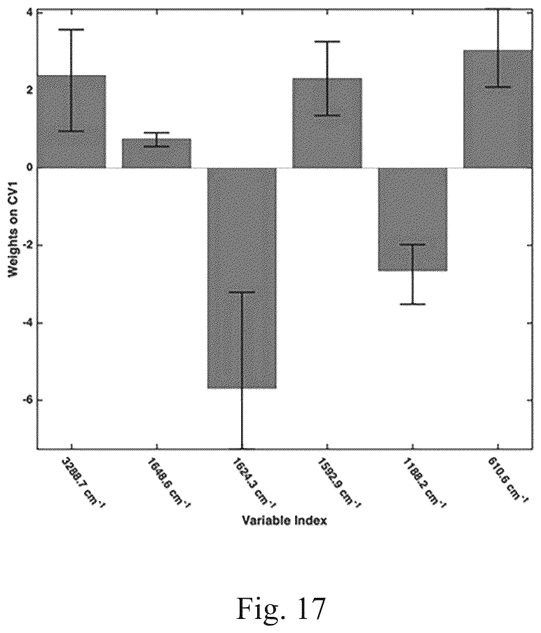

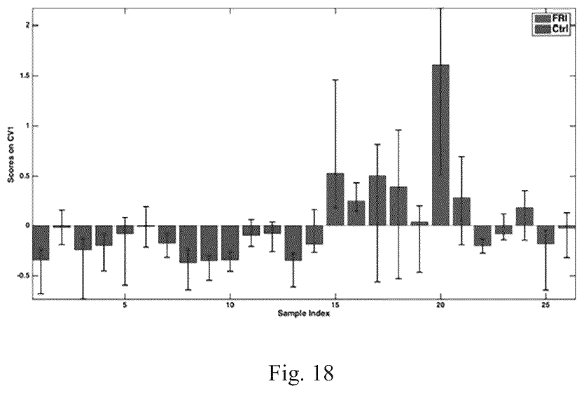

Analysis of the FTIR spectroscopic data, during the model-building phase, resulted in six latent variables (i.e., the six wavenumbers from Example 2B) as optimal complexity (FIG. 15). The overall classification accuracy on the outer loop samples (as defined by the CovSel-PLS-DA model) was found to be 70.9±4.8%, with 69.9±6.2% sensitivity and 71.9±5.9% specificity (FIG. 11). The AUC for this model was 0.761±0.041 indicating an acceptable discriminant ability. Permutation test confirmed that the observed figures of merit were all statistically significant (P<0.05). The results based on comparing the scores of the outer loop samples along the only canonical variate of the model with the variable weights for the construction of that direction are presented in FIG. 16. The scores plot indicated that FRI patients have predominantly negative values, whereas the majority of controls have positive scores. Comparison with the weight values suggests that FRI patients are characterized by higher absorbance at 1624.3 and 1188.2 cm⁻¹ and lower absorbance at 610.6, 1592.9, 1648.6 and 3288.7 cm⁻¹ (FIG. 17).

### 2D. Autoscaling

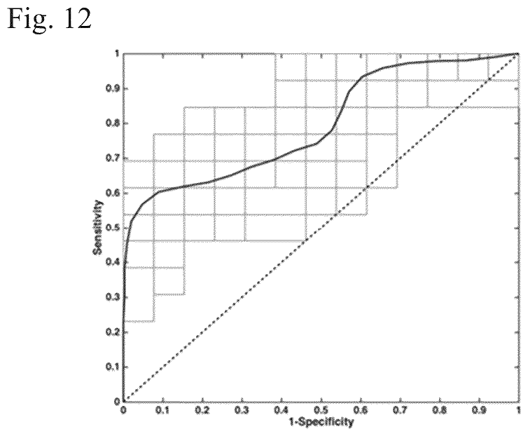

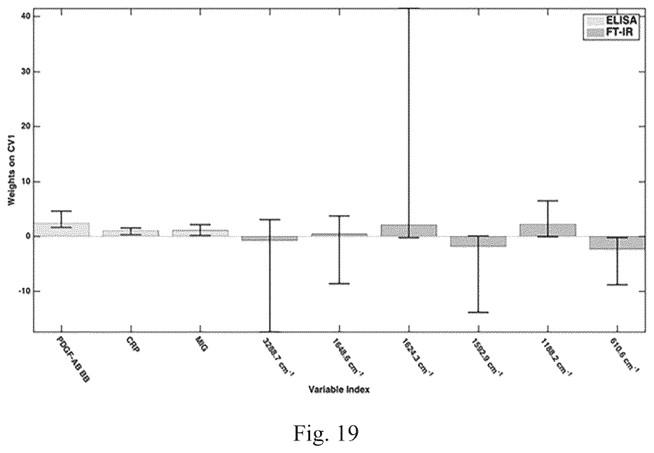

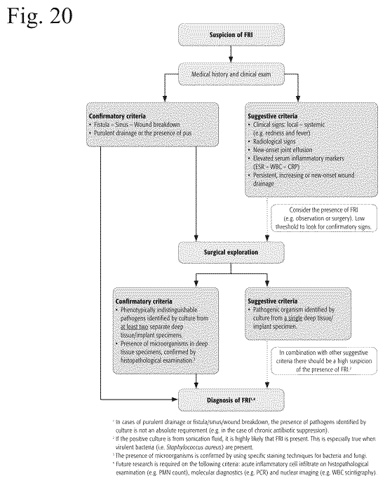

The predictive variables from the previous two Examples (2B and 2C) were autoscaled, and the two blocks of data (proteins and MIR spectra) were further block-scaled to allow equal contributions. The model consistently included four variables (i.e., PDGF-AB/BB, CRP, MIG, and 610.6 cm⁻⁴) that contributed significantly to the model (FIG. 18 and FIG. 19) and provided an overall classification accuracy on the external loop samples of 75.2±4.5%, with 61.5±6.3% sensitivity and 88.9±6.6% specificity (FIG. 12). The AUC was 0.795±0.054; indicating near excellent discriminant ability.

### 2E. Discussion

This is the first study demonstrating significant differences comparing measured values 10 of PDGF-AB/BB and VEGF-A between FRI and control patients. It also confirms that CRP and IL-6 may be useful in the diagnostic work-up of FRI.

Complementary approaches of univariate and multivariate analytical methods were used to show biomarker differences from a large panel of inflammatory proteins and MIR spectral signal in plasma obtained from FRI patients and matched controls. Both analytical approaches identified PDGF-AB/BB and CRP as consistent biomarkers with discriminatory abilities. The multivariate method showed MIG combined with PDGF-AB/BB and CRP to be the minimum number of non-redundant variables that significantly contributed to the final predictive model. Univariate analysis identified IL-6 and VEGF-A to be additional biomarkers that were significantly different between groups. The combination of these two analytical methods provides complementary results that reduce loss of information encountered from either analytical approach. Therefore, the results of each analytical approach also require individual interpretation, rather than an attempt to validate the results of one method against the other. The lack of significance for MIG in the univariate approach may be due to an existing covariance of this plasma protein with PDGF-AB/BB and CRP that is identifiable through the multivariate approach. On the other hand, lack of IL-6 and VEGF-A being selected in the multivariate analysis may be due to less significant correlation/covariance between these two and other selected biomarkers. However, this does not imply that these two biomarkers do not have significant differences between the two groups, but that the combination of PDGF-AB/BB, CRP, MIG variables was the minimum number of non-redundant variables that was able to best demonstrate the group differences in the multivariate approach in this cohort.

There is limited literature on novel methods for diagnosis of FRIs. Historically, the standard inflammatory markers used to aide in the diagnosis of FRI have been peripheral WBC, CRP, and ESR. A systematic review of diagnostic accuracy of these “classic” plasma inflammatory markers determined that they are insufficient. In that review, sensitivity and specificity based on CRP ranged from 60-100% and 34-86%, respectively. The ability to predict FRI based on PDGF-AB/BB, CRP, MIG in this study was comparable in sensitivity to previous reports based on CRP alone but significantly improved for specificity and accuracy. The predictive model based on IR spectra variables showed similar sensitivity to that of the select protein biomarkers. However, despite having an acceptable discriminatory ability, the specificity and accuracy were lower than those based on protein biomarkers. Combining the selected protein biomarkers and spectral variables from each model improved the discriminatory ability of the final predictive model compared to spectral data alone, but it did not surpass the performance of the model based on protein biomarkers alone. These results suggest that, in this cohort of patients, the predictive model based on this select panel of protein biomarkers is the more accurate and specific discriminatory tool, with similar sensitivity compared to spectral fingerprint alone. The comparable results based on MIR spectral data demonstrate the potential ability of this FTIR spectroscopy method to be used as a surrogate for this protein panel as a potential point of care diagnostic screening tool. Advantages of using FTIR spectroscopy of dried films compared to ELISA-based biomarkers include lack of need for adjuvants and cost effectiveness (˜5% the cost of ELISA methods).

### Example 3. Plasma Metabolomics Discriminates Patients with Fracture-Related Infection from Uninfected Controls

Fracture related infection (FRI) is a devastating complication of orthopaedic trauma. Defined as the presence of pathogenic microorganisms in the soft tissues, bone, or implants around the site of a fracture, FRI causes increased morbidity for patients in the form of additional surgeries, prolonged hospitalization, lengthy intravenous antibiotic treatments, and often significant declines in function and quality of life. Globally, approximately 1.8 million FRIs are estimated to occur every year (Metsemakers, Moriarty, Morgenstern, Marais, Onsea, O'Toole, Depypere et al. 2023). Costs for each of these cases are estimated to be three to seven times that of a non-infected fracture (Haidari, Buijs, Plate, Zomer, FFA, Hietbrink, Govaert 2024). Despite the high burden, confirming the presence of FRI remains a challenge, particularly without invasive surgery.

Serum inflammatory markers, namely white blood cell count (WBC), erythrocyte sedimentation rate (ESR), and C-reactive protein (CRP) are widely used in clinical practice but have limited diagnostic accuracy for FRI. Cultures of intra-operative tissue samples and swabs are often obtained, but results can take up to 14 days to finalize and are dependent on sample quality. Further, in current clinical practice, patients with low-grade infection may not even raise enough clinical suspicion for these diagnostic tests and surgeries to be performed. As such, there is a pressing need for rapid, non-invasive means of diagnosing FRI.

Metabolomics has emerged as a powerful tool for novel biomarker-based diagnostic tests. However, it has yet to be studied in the setting of FRI. In this discovery study, the serum plasma metabolomic profile of orthopaedic trauma patients with and without confirmed FRI was investigated. It was hypothesized that metabolomic differences exist between these two groups, and that metabolomic biomarkers would classify FRI at a rate equal to or greater than conventional clinical biomarkers.

### Methods Patients

This study was approved by the local Institutional Review Board. All patients included provided informed consent. Patients were eligible if they were aged 18 or older and sustained an extremity or pelvic fracture surgically treated at a Level 1 Trauma institution with a retained orthopaedic implant. Exclusion criteria were as detailed in previous proteomics studies on FRI patients.

In total, 54 patients, with blood draws dated from June 2019 to April 2024, were included in the analysis. Twenty-seven FRI patients and 27 control patients were analyzed. Classification was based on criteria for FRI diagnosis proposed by the international FRI Consensus Group formed in 2016 (Metsemakers, Morgenstern, McNally, Moriarty, McFadyen, Scarborough, Athanasou et al. 2018). FRI patients were identified as those meeting either, 1.) confirmatory criteria on clinical exam, or 2.) suggestive criteria on clinical exam followed by confirmatory criteria from intra-operative findings (FIG. 20). For FRI patients with positive intra-operative cultures, the presence of mono- or polymicrobial growth was also recorded.

Control patients were matched to FRI patients based on age, fracture region and time since index surgery. Age was matched to within 15 years. Fracture region was matched to one of four categories: upper extremity long bones (clavicle, humerus, and/or radius/ulna), pelvis or acetabulum, lower extremity long bones (femur and/or tibia), and other lower extremity fractures (patella, ankle, and/or tarsal bones). Time of blood draw was matched to within two weeks of the index fracture surgery. Once an FRI patient was confirmed, the institution's orthopaedic clinic schedules were searched for an eligible matched control patient based on the above criteria. Once approached and enrolled in the study, the patient eventually qualified as a control patient provided they had no suggestive or confirmed FRI criteria within six months of their fracture surgery.

### Sample Preparation

Five milliliters of peripheral venous blood was obtained from FRI and control patients. Blood draws for FRI patients were obtained on the day of infection surgery (24/27) or within one week prior to infection surgery (3/27). All blood draws for control patients were obtained in clinic at their time-matched routine follow-up visit. The blood samples were collected in EDTA tubes, inverted four to five times to mix the blood and anticoagulants, and centrifuged at 1500 g for ten minutes. Plasma was then extracted, aliquoted, and stored at −80° C. until analysis.

### Metabolomic Analysis

Untargeted metabolomic analysis of 1453 metabolites was performed on FRI and control patient plasma samples using ultra-performance liquid chromatography and tandem mass spectroscopy (Metabolon, Inc. Morrisville, NC, USA). Further details of Metabolon's protocol can be found in the Supplementary Content 1 section. All samples were run in the same batch.

### Statistical Analysis

Matched pairs t-tests were performed to identify metabolites that were significantly different (relative quantitation) between FRI patients and controls, as measured by normalized, log-transformed mean scaled intensity. P-values less than 0.05 were considered significant. To control for false positive results in the setting of multiple testing, q-values were also calculated and used to further narrow down the list of notable metabolites.

For subgroup analysis, Welch's t-tests were performed to identify metabolites that were significantly different between FRI patients that had positive versus negative operative cultures and between FRI patients whose positive cultures were monomicrobial versus polymicrobial.

Finally, a random forest algorithm based on all detected metabolites was built to predict the presence of FRI. Metabolites were ranked in importance based on mean percent decrease in accuracy.

### Results

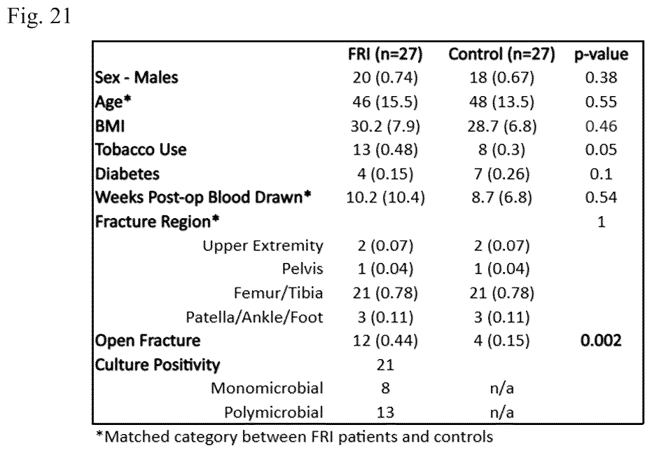

All twenty-seven patients in the FRI group ended up meeting confirmatory criteria pre-operatively, as defined by purulent drainage (n=23) and/or a fistula, sinus, or wound breakdown (n=16). Six patients had culture negative FRI. FIG. 21 compares patient demographic, comorbidity, and injury characteristics between the FRI and uninfected control groups.

In total, 1453 metabolites were analyzed, 1182 identified and 271 unnamed. A full list of metabolites, organized by biochemical class, is included in the Supplementary Content 2 section. Matched pairs t-testing identified 248 significantly different metabolites between FRI and control serum (103 upregulated, 145 downregulated). Of these, 83 had a q-value less than 0.05.

Welch's t-test comparing the plasma from culture positive and culture negative FRI patients found 55 significantly upregulated and 55 significantly downregulated metabolites. Welch's t-test comparing the plasma of the 13 polymicrobial FRI patients and the 8 monomicrobial FRI patients (3 with methicillin-sensitive Staph. aureus, 2 with methicillin-resistant Staph. aureus, 2 with Pseudomonas, and one with Staph. lugdunensis) found 36 significantly upregulated and 21 significantly downregulated metabolites.

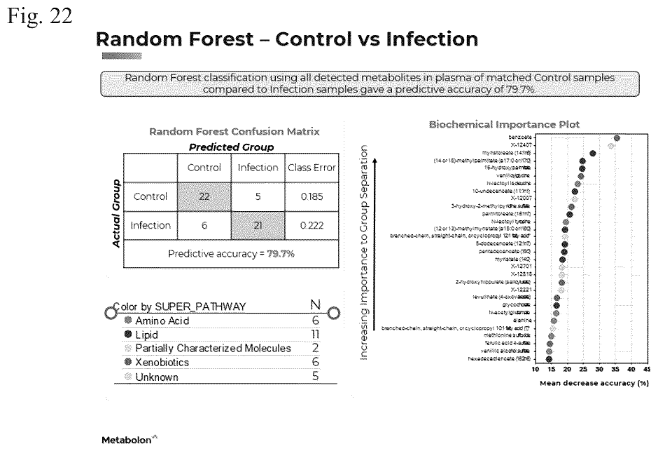

Finally, the random forest algorithm incorporating all detected metabolites had a predictive accuracy of 79.7% (95% CI: 66.5-89.4%), sensitivity of 77.8% (95% CI: 57.7-91.4), and 5 specificity of 81.5% (95% CI: 61.9-93.7%). The top 30 metabolites contributing to this algorithm, as measured by mean percent decrease in accuracy, are listed in FIG. 22. Notable, five of the top 30 biomarker candidates in the RFA are xenobiotic metabolites, including the top candidate, benzoate. Table 6 lists the 26 metabolites from the top 30 in the RFA that also have q-values <0.05.

TABLE 6
26 Metabolites from the RFA top 30 that also have q-value < 0.05

benzoate
X-12407
myristoleate (14:1n5)
(14 or 15)-methylpalmitate (a17:0 or i17:0)
16-hydroxypalmitate
vanilloylglycine
N-lactoyl isoleucine
X-12007
3-hydroxy-2-methylpyridine sulfate
palmitoleate (16:1n7)
N-lactoyl tyrosine
(12 or 13)-methylmyristate (a15:0 or i15:0)
branched-chain, straight-chain, or cyclopropyl 12:1 fatty acid
5-dodecenoate (12:1n7)
pentadecanoate (15:0)
myristate (14:0)
X-12701
X-12818
X-12221
levulinate (4-oxovalerate)
N-acetylglutamate
alanine
branched-chain, straight-chain, or cyclopropyl 10:1 fatty acid (1)*
ferulic acid 4-sulfate
vanillic alcohol sulfate
hexadecadienoate (16:2n6)

Metabolomics is a burgeoning field in molecular diagnostics, with applications ranging from the diagnosis of sepsis to cancer. However, it is understood that the plasma metabolome has never been clinically investigated in the setting of FRI.

A pilot study comparing FRI patients to controls yielded hundreds of differentially present serum metabolites. Metabolic pathway-level analyses, as shown in the below section, revealed the following trends in FRI patients: 1.) increased free fatty acids (possibly from increased energy demand and/or from fasting), 2.) decreased cell membrane phospholipids (indicating decreased cell membrane remodeling), 3.) decreased dietary aromatic amino acid metabolites (suggesting an altered gut microbiome and/or nutrition state) and 4.) decreased primary bile acids. While the significance of these pathway level differences can only be speculated upon at this point, taken together, the accuracy for FRI classification based on the RFA of all detected metabolites was nearly 80% (sensitivity 81% and specificity 79%). Per a recent metanalysis, this is an improvement compared to conventionally used biomarkers, namely WBC (51.7% sensitivity and 67.1% specificity), ESR (45.1% sensitivity and 79.3% specificity), and even CRP (77% sensitivity and 67.9% specificity).

A multi-omics approach to optimize diagnostic accuracy of FRI was envisioned. Recent proteomics study in a nearly identical group of FRI patients and controls as this study identified 32 differentially expressed proteins, primarily in the complement and coagulation cascades. Taken together, this led to an 88.9% specificity compared to CRP's 74.1%.

This study suggested metabolomic differences between culture positive and culture negative FRI patients, and furthermore between polymicrobial and monomicrobial patients. Larger studies may be able to identify more clinically applicable insights from such sub-analyses. Metabolomic differences between gram positive and gram negative FRI patients may be clinically relevant to rapidly inform empiric antibiotic treatment. An a priori power analysis was not performed here. Finally, control for patients' time of last oral intake or what was consumed before blood draw was not controlled.

This Example suggests that the plasma metabolomic and proteomic profiles of FRI and control patients significantly differ. Random forest analysis resulted in a FRI diagnostic accuracy of 80%, which is greater than conventional biomarkers. Further studies with larger sample sizes and integration of multi-omic testing are warranted.

### Supplementary Content 1

### Metabolon Platform

Sample Accessioning: Following receipt, samples were inventoried and immediately stored at −80° C. Each sample received was accessioned into the Metabolon Laboratory Information Management System (LIMS) system and was assigned by the LIMS a unique identifier that was associated with the original source identifier only. This identifier was used to track all sample handling, tasks, results, etc. The samples (and all derived aliquots) were tracked by the LIMS system. All portions of any sample were automatically assigned their own unique identifiers by the LIMS when a new task was created; the relationship of these samples was also tracked. All samples were maintained at −80° C. until processed.

Sample Preparation: Samples were prepared using the automated MicroLab STAR® system from Hamilton Company. Several recovery standards were added prior to the first step in the extraction process for QC purposes. To remove protein, dissociate small molecules bound to protein or trapped in the precipitated protein matrix, and to recover chemically diverse metabolites, proteins were precipitated with methanol under vigorous shaking for 2 min (Glen Mills GenoGrinder 2000) followed by centrifugation. The resulting extract was divided into multiple fractions: two for analysis by two separate reverse phase (RP)/UPLC-MS/MS methods with positive ion mode electrospray ionization (ESI), one for analysis by RP/UPLC-MS/MS with negative ion mode ESI, one for analysis by HILIC/UPLC-MS/MS with negative ion mode ESI, while the remaining fractions were reserved for backup. Samples were placed briefly on a TurboVap® (Zymark) to remove the organic solvent. The sample extracts were stored overnight under nitrogen before preparation for analysis.

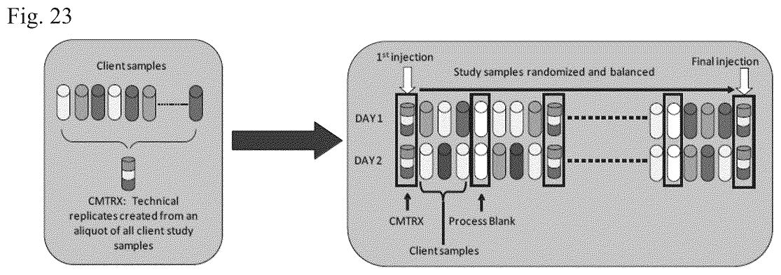

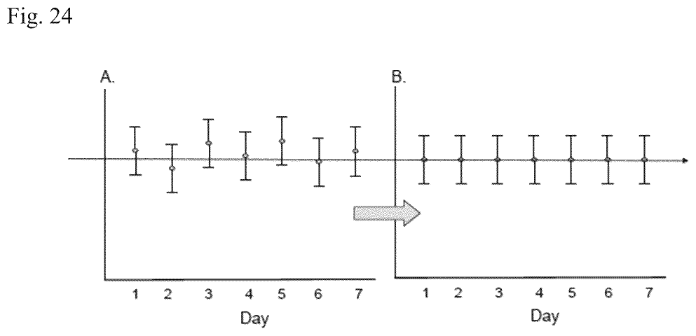

QA/QC: Several types of controls were analyzed in concert with the experimental samples: a pooled matrix sample generated by taking a small volume of each experimental sample (or alternatively, use of a pool of well-characterized human plasma) served as a technical replicate throughout the data set; extracted water samples served as process blanks; and a cocktail of QC standards that were carefully chosen not to interfere with the measurement of endogenous compounds were spiked into every analyzed sample, allowed instrument performance monitoring and aided chromatographic alignment. Tables 7 and 8 describe these QC samples and standards. Instrument variability was determined by calculating the median relative standard deviation (RSD) for the standards that were added to each sample prior to injection into the mass spectrometers. Overall process variability was determined by calculating the median RSD for all endogenous metabolites (i.e., non-instrument standards) present in 100% of the pooled matrix samples. Experimental samples were randomized across the platform run with QC samples spaced evenly among the injections, as outlined in FIG. 23.

TABLE 7
Description of Metabolon QC Samples

| Type | Description | Purpose |
|---|---|---|
| MTRX | Large pool of human plasma | Assure that all aspects of the Metabolon |
| | maintained by Metabolon that has | process are operating within |
| | been characterized extensively. | specifications. |
| CMTRX | Pool created by taking a small | Assess the effect of a non-plasma |
| | aliquot from every customer | matrix on the Metabolon process and |
| | sample. | distinguish biological variability from |
| | | process variability. |
| PRCS | Aliquot of ultra-pure water | Process Blank used to assess the |
| | | contribution to compound signals from |
| | | the process. |

TABLE 8
Metabolon QC Standards

| Type | Description | Purpose |
|---|---|---|
| RS | Recovery Standard | Assess variability and verify performance of |
| | | extraction and instrumentation. |
| IS | Internal Standard | Assess variability and |
| | | performance of instrument. |

### Ultrahigh Performance Liquid Chromatography-Tandem Mass Spectroscopy

(UPLC-MS/MS): All methods utilized a Waters ACQUITY ultra-performance liquid chromatography (UPLC) and a Thermo Scientific Q-Exactive high resolution/accurate mass spectrometer interfaced with a heated electrospray ionization (HESI-II) source and Orbitrap mass analyzer operated at 35,000 mass resolution (PMID: 32445384). The dried sample extract were then reconstituted in solvents compatible to each of the four methods. Each reconstitution solvent contained a series of standards at fixed concentrations to ensure injection and chromatographic consistency. One aliquot was analyzed using acidic positive ion conditions, chromatographically optimized for more hydrophilic compounds (PosEarly). In this method, the extract was gradient eluted from a C18 column (Waters UPLC BEH C18-2.1×100 mm, 1.7 m) using water and methanol, containing 0.05% perfluoropentanoic acid (PFPA) and 0.1% formic acid (FA). Another aliquot was also analyzed using acidic positive ion conditions, however it was chromatographically optimized for more hydrophobic compounds (PosLate). In this method, the extract was gradient eluted from the same aforementioned C18 column using methanol, acetonitrile, water, 0.05% PFPA and 0.01% FA and was operated at an overall higher organic content. Another aliquot was analyzed using basic negative ion optimized conditions using a separate dedicated C18 column (Neg). The basic extracts were gradient eluted from the column using methanol and water, however with 6.5 mM Ammonium Bicarbonate at pH 8. The fourth aliquot was analyzed via negative ionization following elution from a HILIC column (Waters UPLC BEH Amide 2.1×150 mm, 1.7 μm) using a gradient consisting of water and acetonitrile with 10 mM Ammonium Formate, pH 10.8 (HILIC). The MS analysis alternated between MS and data-dependent MSn scans using dynamic exclusion. The scan range varied slightly between methods but covered 70-1000 m/z. Raw data files are archived and extracted as described below.

Bioinformatics: The informatics system consisted of four major components, the LIMS, the data extraction and peak identification software, data processing tools for QC and compound identification, and a collection of information interpretation and visualization tools for use by data analysts. The hardware and software foundations for these informatics components were the LAN backbone, and a database server running Oracle 10.2.0.1 Enterprise Edition.

LIMS: The purpose of the Metabolon LIMS system was to enable fully auditable laboratory automation through a secure, easy to use, and highly specialized system. The scope of the Metabolon LIMS system encompasses sample accessioning, sample preparation and instrumental analysis and reporting and advanced data analysis. All of the subsequent software systems are grounded in the LIMS data structures. It has been modified to leverage and interface with the in-house information extraction and data visualization systems, as well as third party instrumentation and data analysis software.

Data Extraction and Compound Identification: Raw data was extracted, peak identified and QC processed using a combination of Metabolon developed software services (applications). Each of these services perform a specific task independently, and they communicate/coordinate with each other using industry-standard protocols. Compounds were identified by comparison to library entries of purified standards or recurrent unknown entities. Metabolon maintains a library based on authenticated standards that contains the retention time/index (RI), mass to charge ratio (m z), and fragmentation data on all molecules present in the library. Furthermore, biochemical identifications are based on three criteria: retention index within a narrow RI window of the proposed identification, accurate mass match to the library+/−10 ppm, and the MS/MS forward and reverse scores between the experimental data and authentic standards. The MS/MS scores are based on a comparison of the ions present in the experimental spectrum to the ions present in the library spectrum. While there may be similarities between molecules based on one of these factors, the use of all three data points is utilized to distinguish and differentiate biochemicals. More than 5,400 commercially available purified or in-house synthesized standard compounds have been acquired and analyzed on all platforms for determination of their analytical characteristics. An additional 7000 mass spectral entries have been created for structurally unnamed biochemicals, which have been identified by virtue of their recurrent nature (both chromatographic and mass spectral). These compounds have the potential to be identified by future acquisition of a matching purified standard or by classical structural analysis. Metabolon continuously adds biologically-relevant compounds to its chemical library to further enhance its level of Tier 1 metabolite identifications.

Compound Quality Control: A variety of curation procedures were carried out to ensure that a high-quality data set was made available for statistical analysis and data interpretation. The QC and curation processes were designed to ensure accurate and consistent identification of true chemical entities, and to remove or correct those representing system artifacts, mis-assignments, mis-integration and background noise. Metabolon data analysts use proprietary visualization and interpretation software to confirm the consistency of peak identification and integration among the various samples.

Metabolite Quantification and Data Normalization: Peaks were quantified using area-under-the-curve. For studies spanning multiple days, a data normalization step was performed to correct variation resulting from instrument inter-day tuning differences. Essentially, each compound was corrected in run-day blocks by registering the medians to equal one (1.00) and normalizing each data point proportionately (termed the “block correction”; FIG. 24). For studies that did not require more than one day of analysis, no normalization is necessary, other than for purposes of data visualization. In certain instances, biochemical data may have been normalized to an additional factor (e.g., cell counts, total protein as determined by Bradford assay, osmolality, etc.) to account for differences in metabolite levels due to differences in the amount of material present in each sample.

### Supplementary Content 2

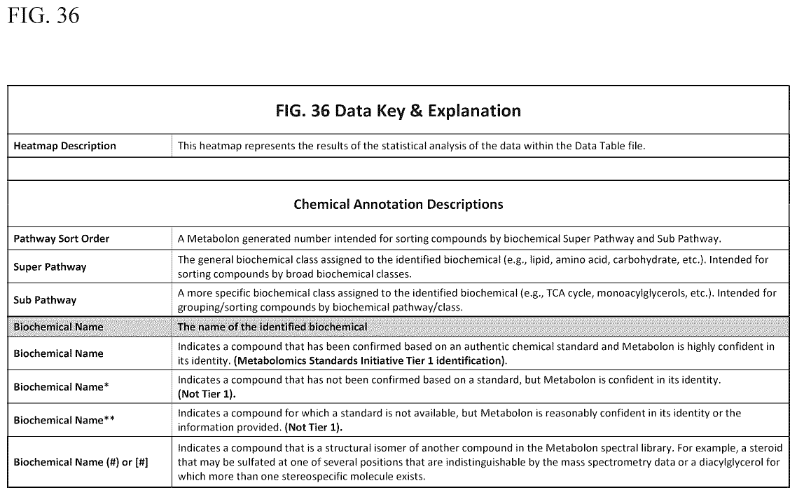

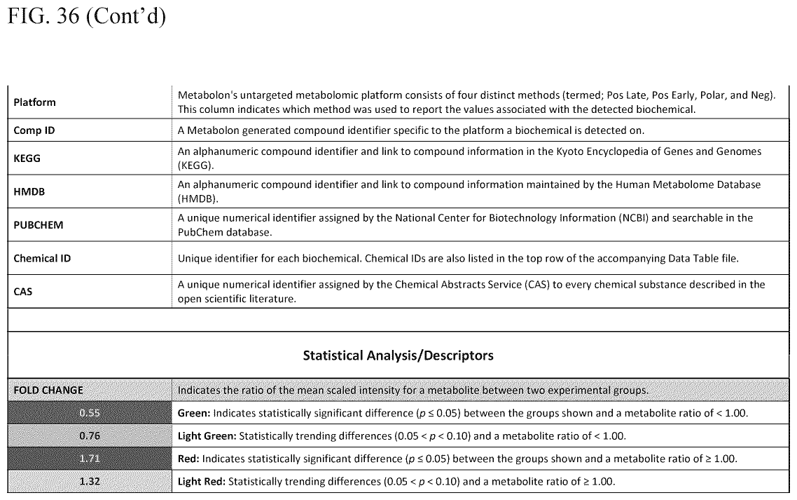

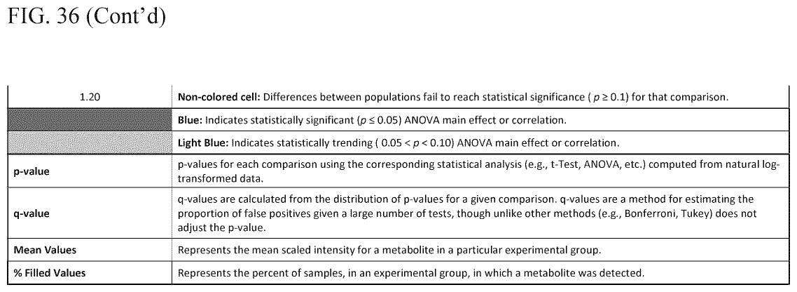

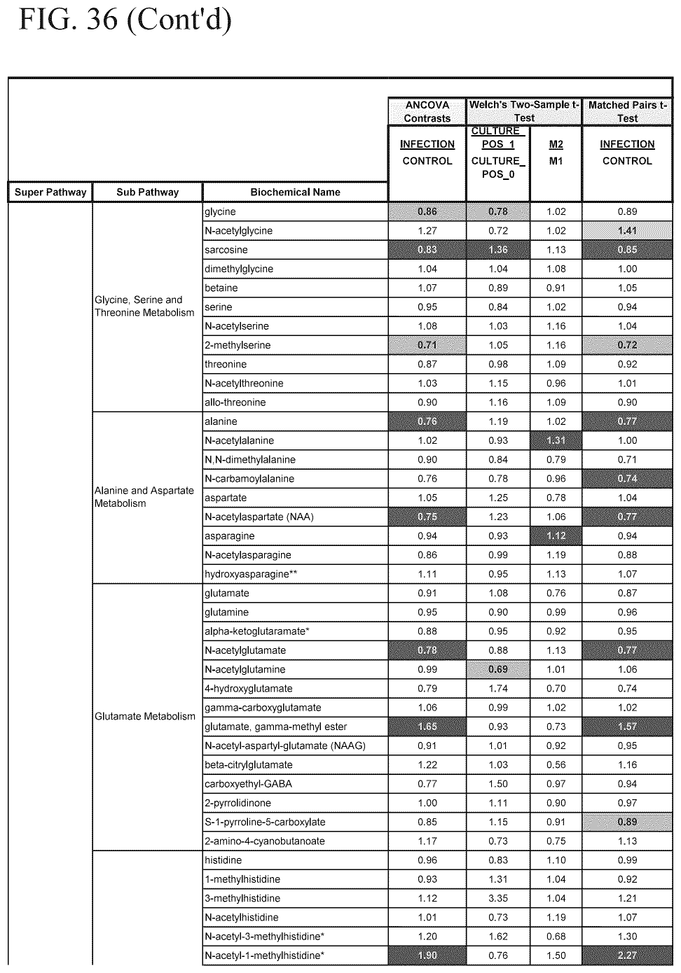

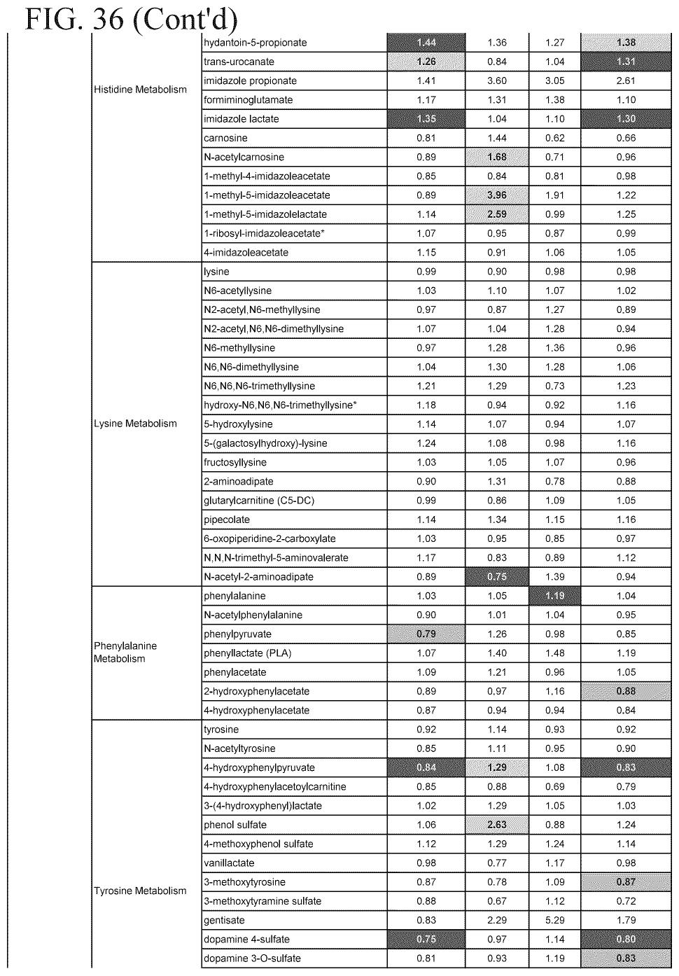

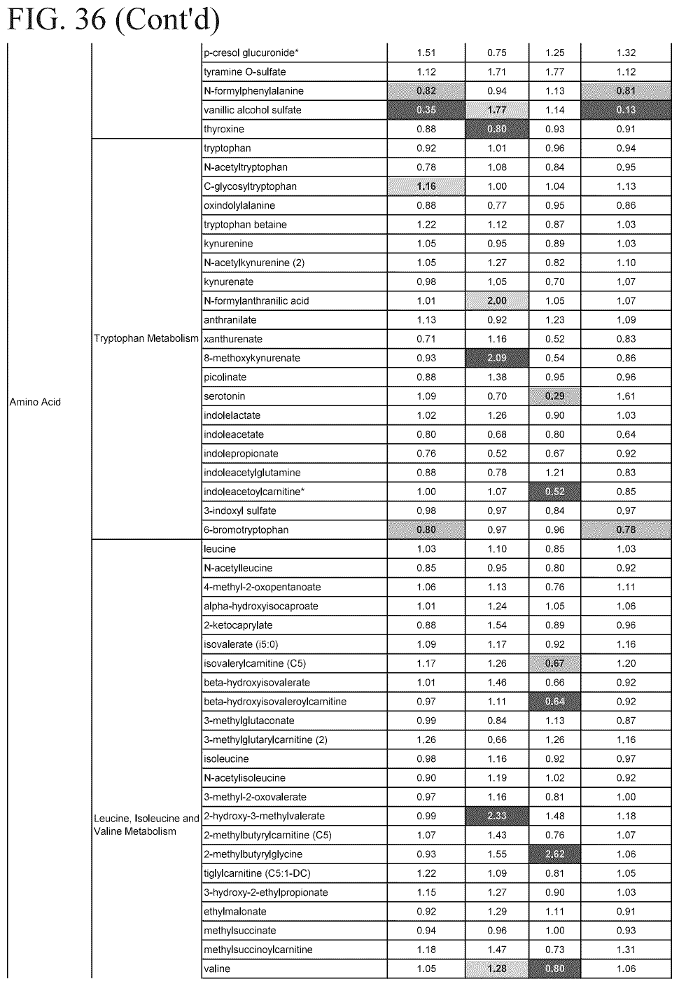

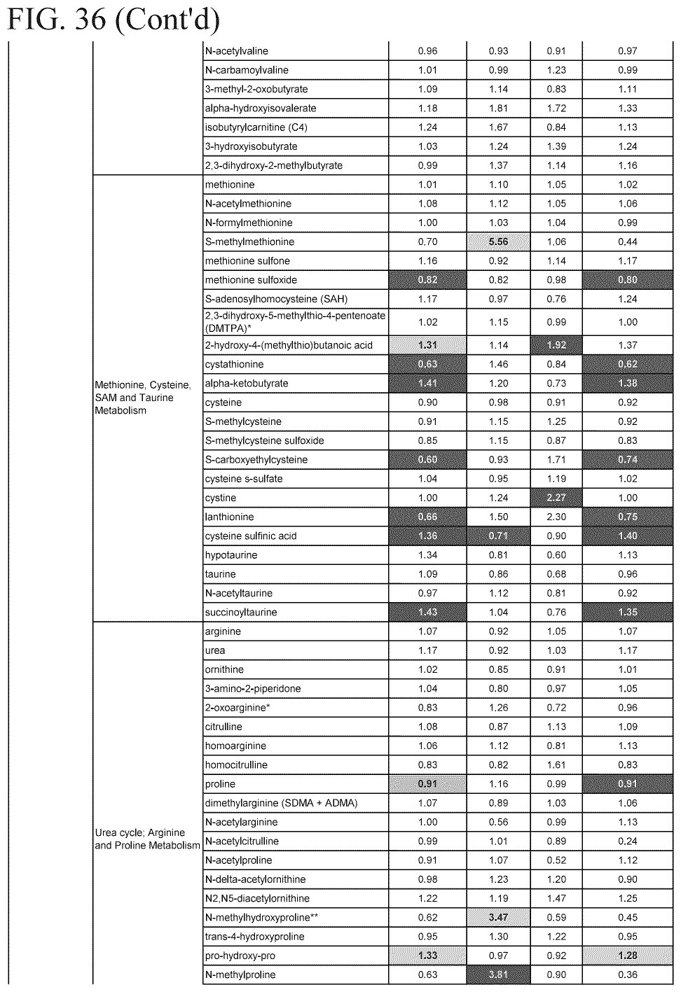

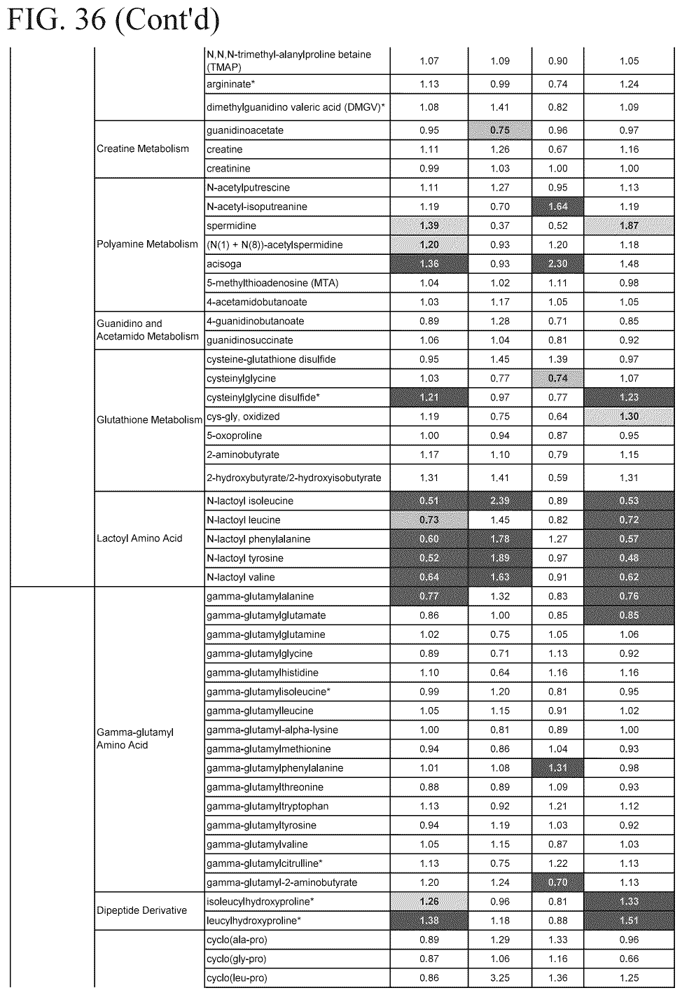

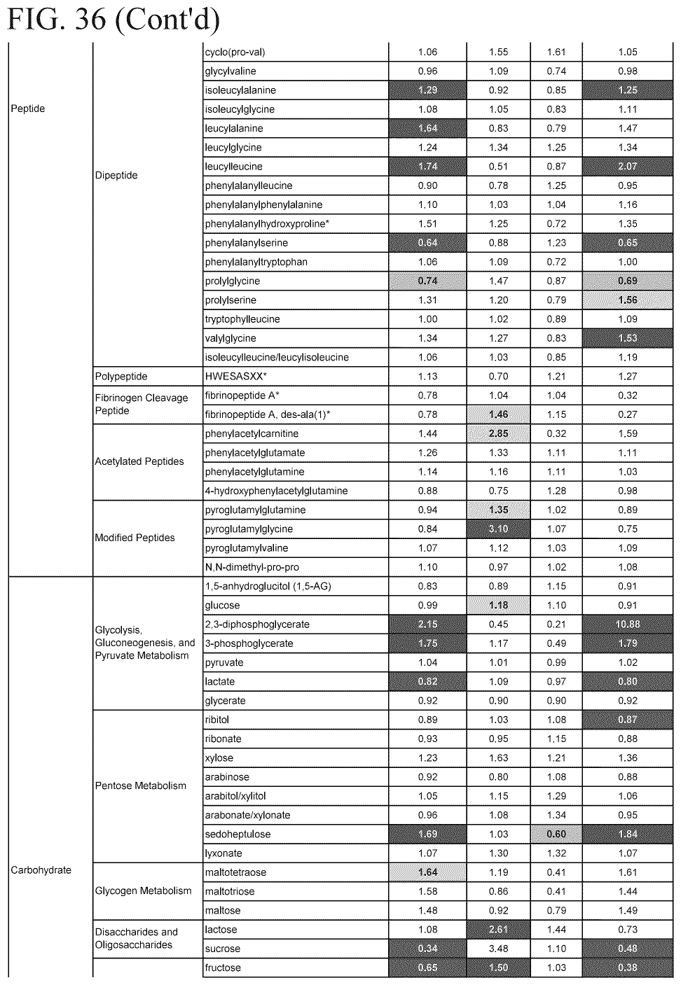

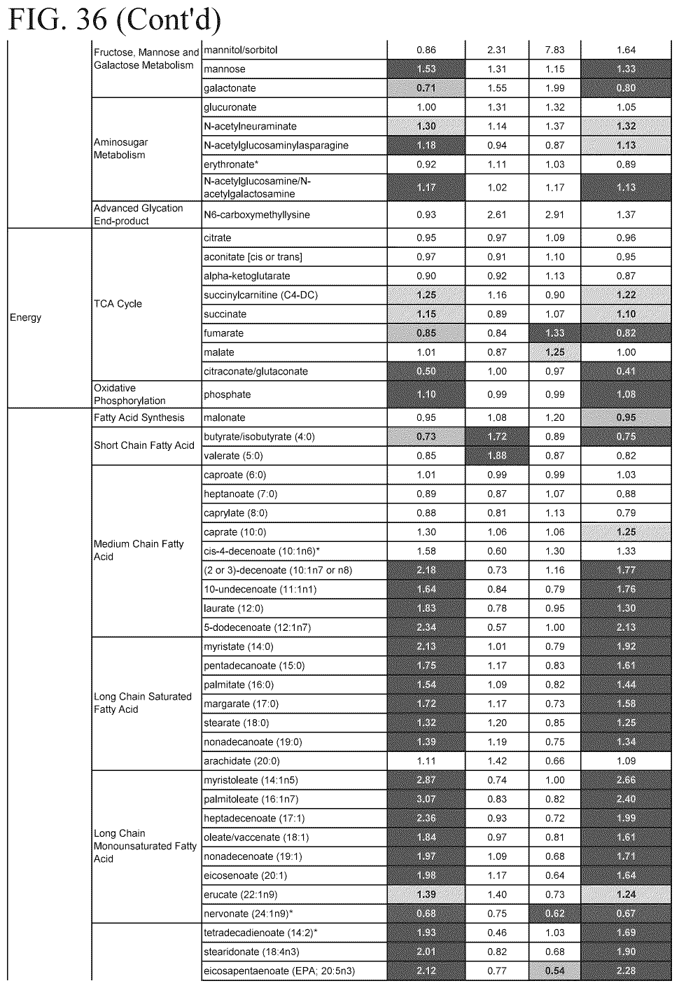

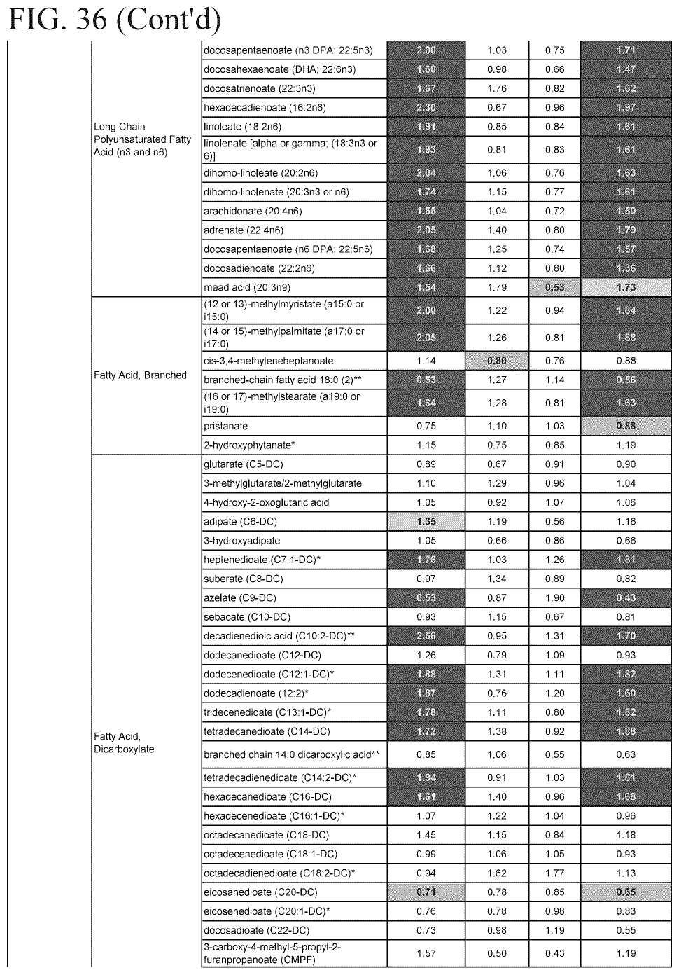

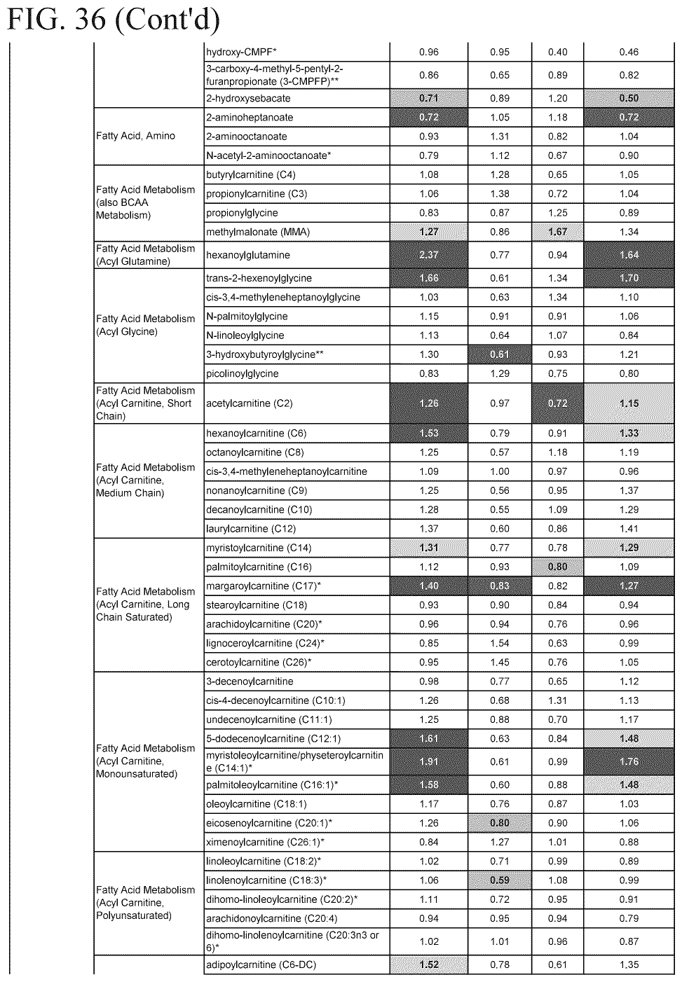

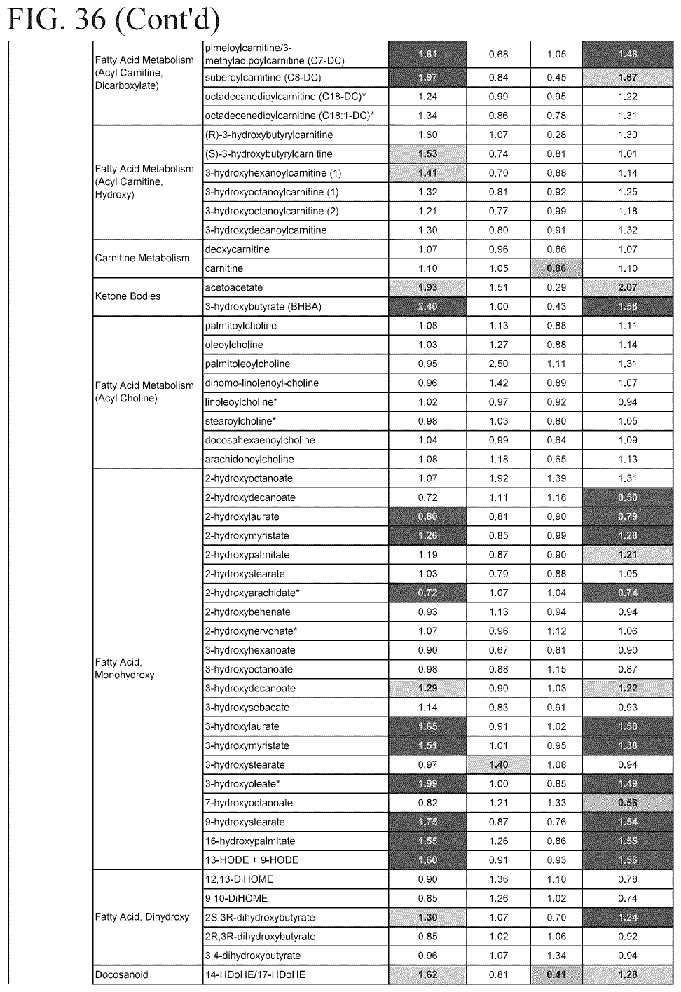

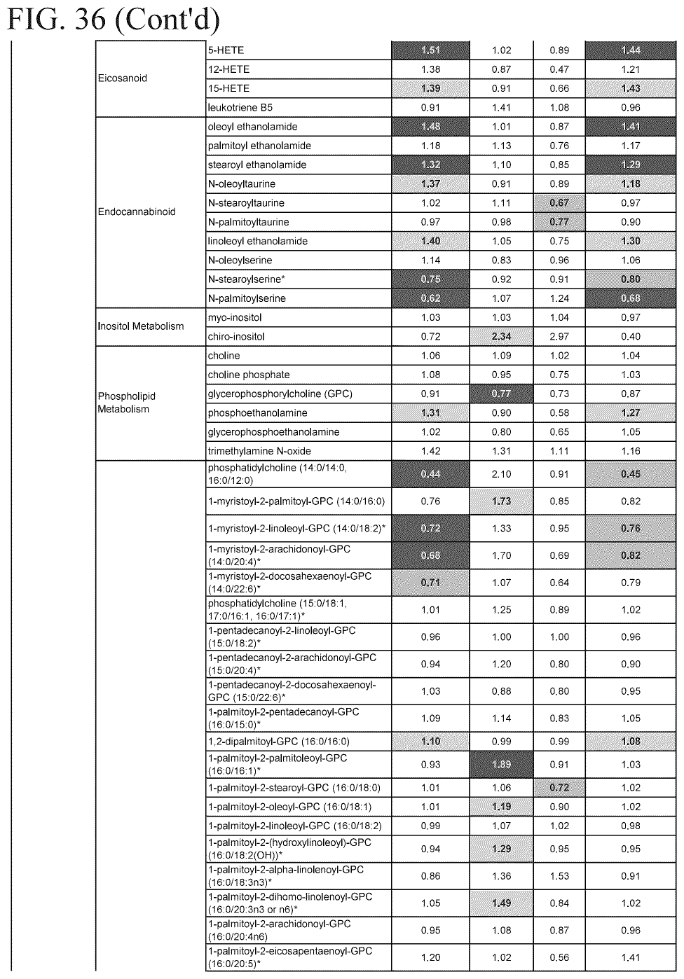

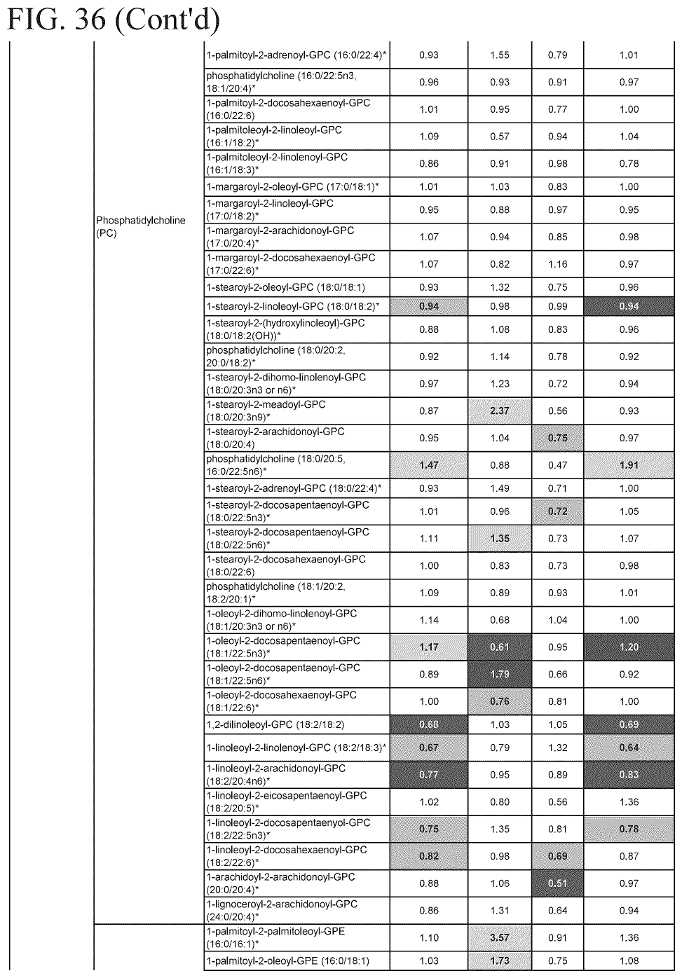

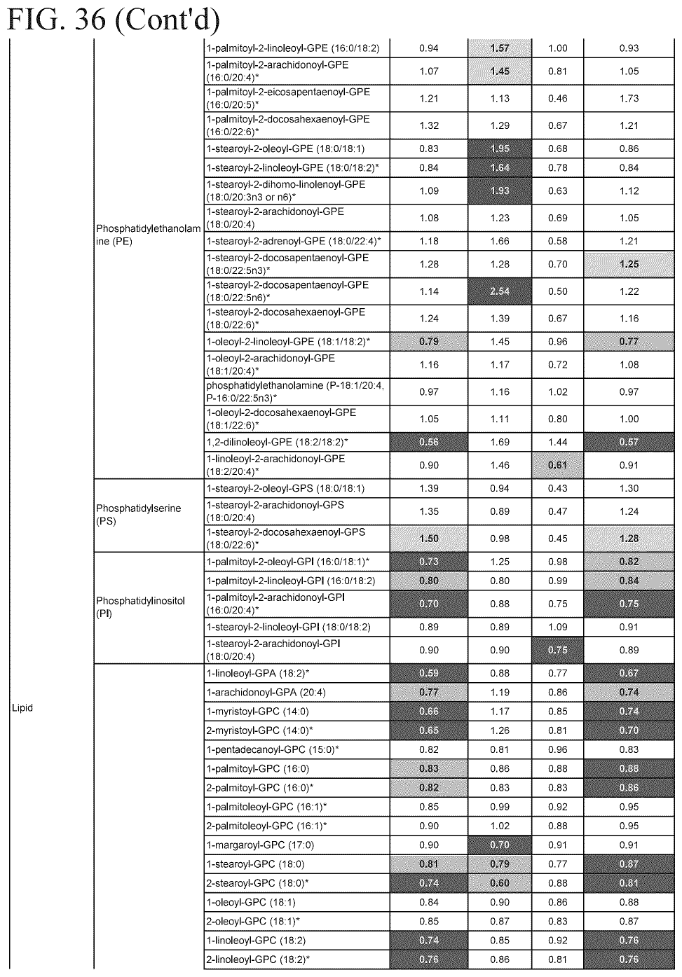

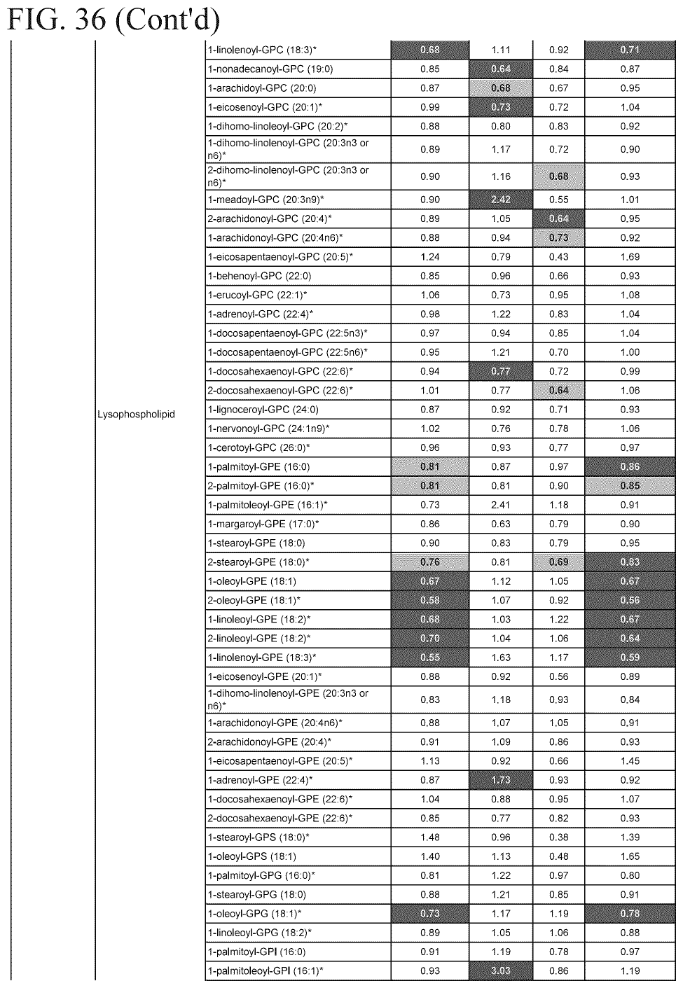

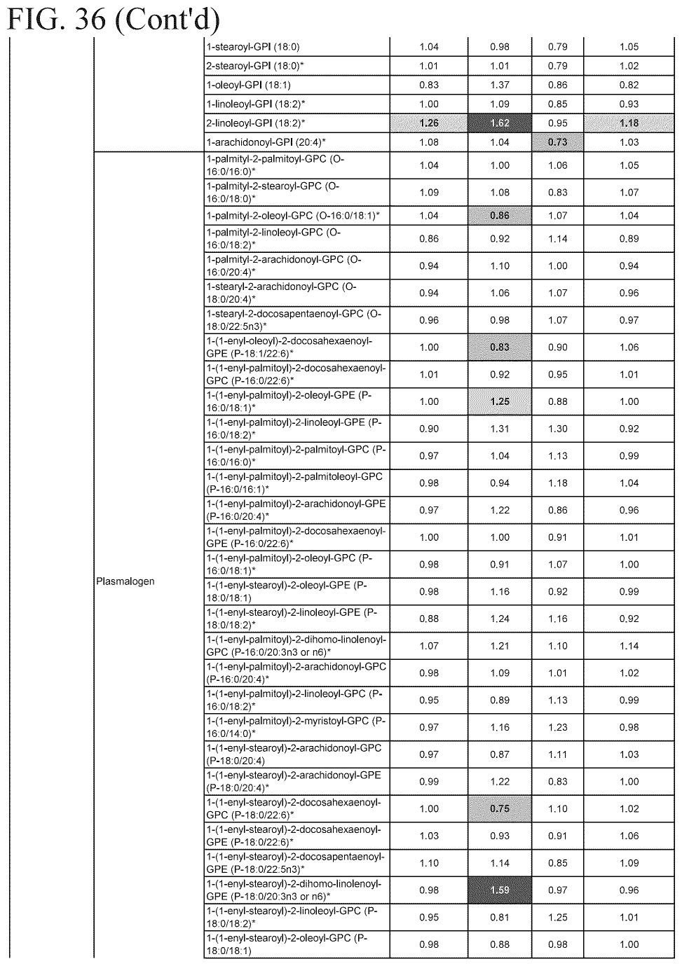

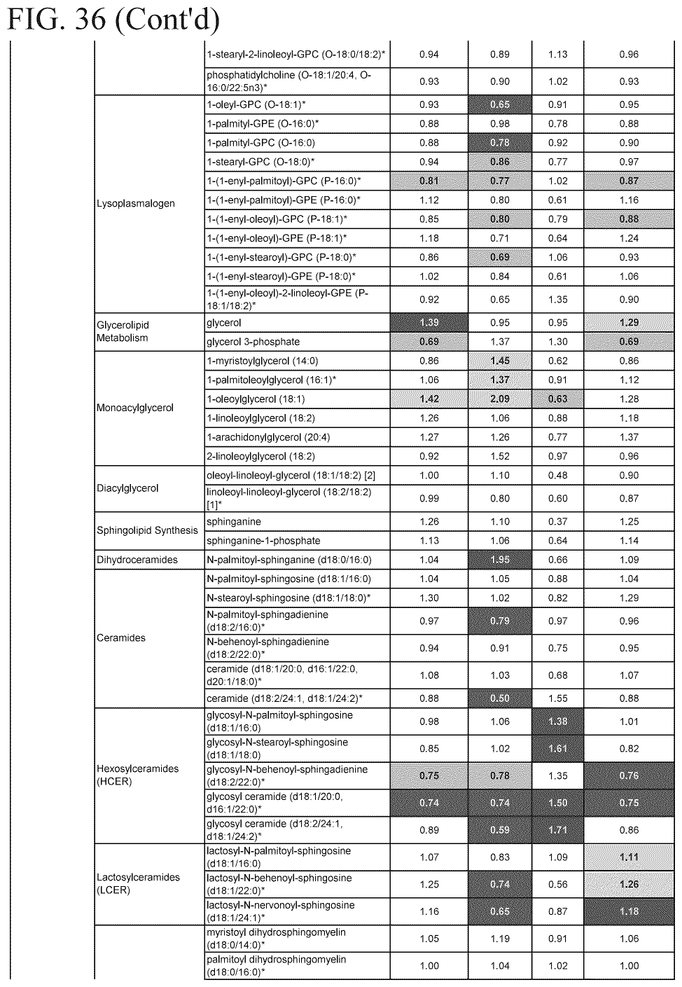

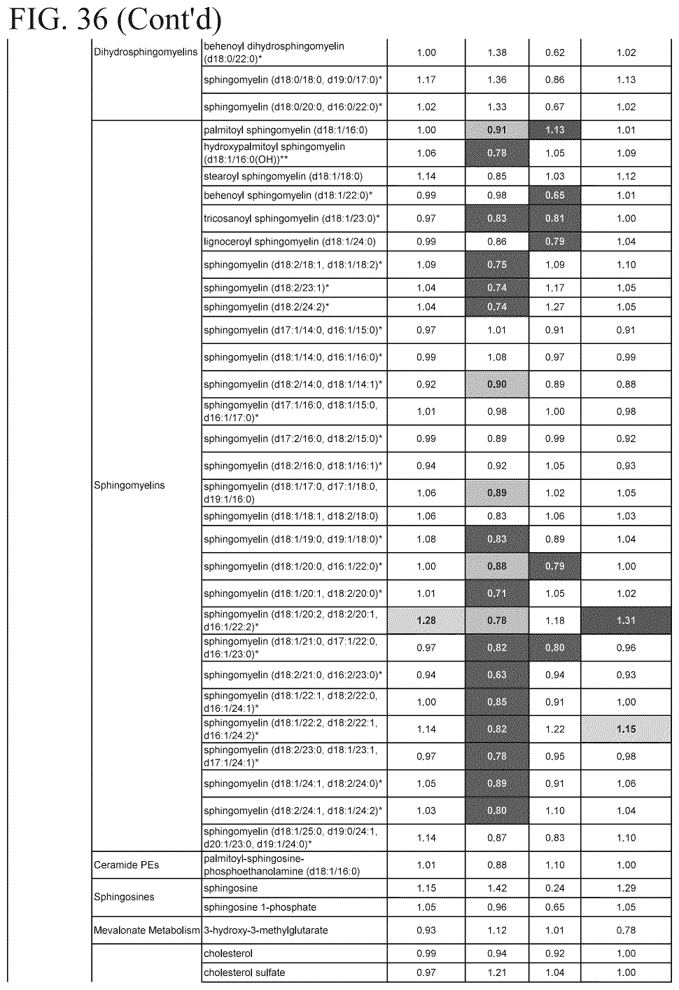

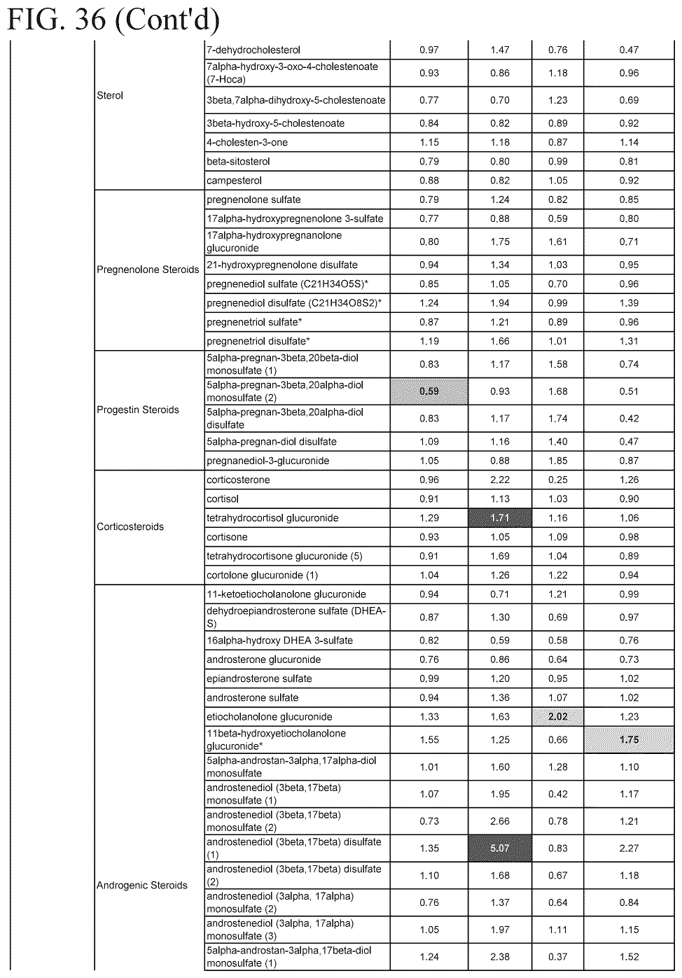

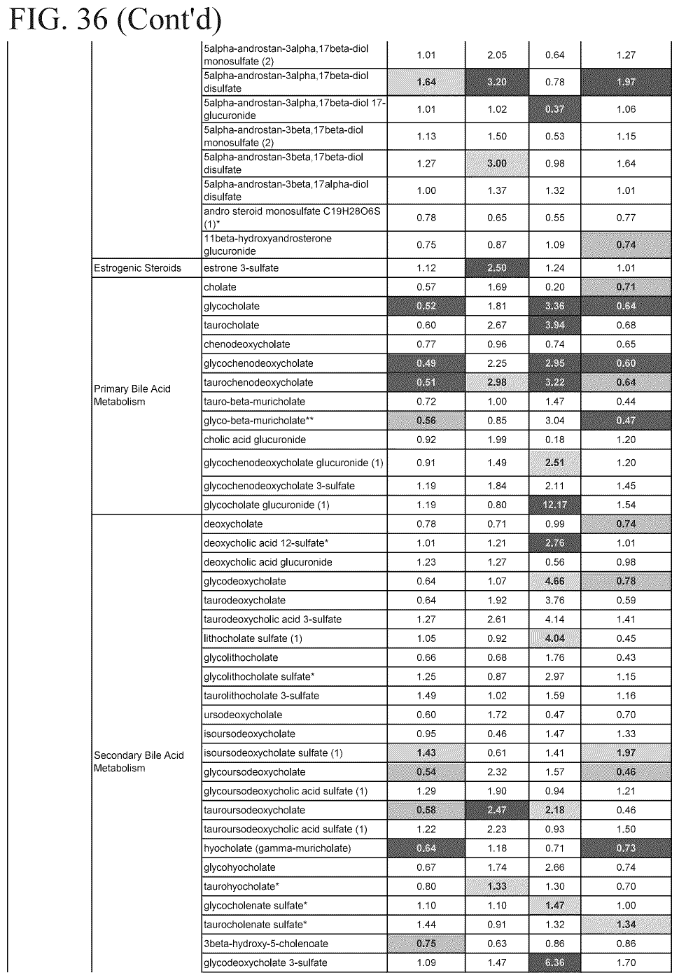

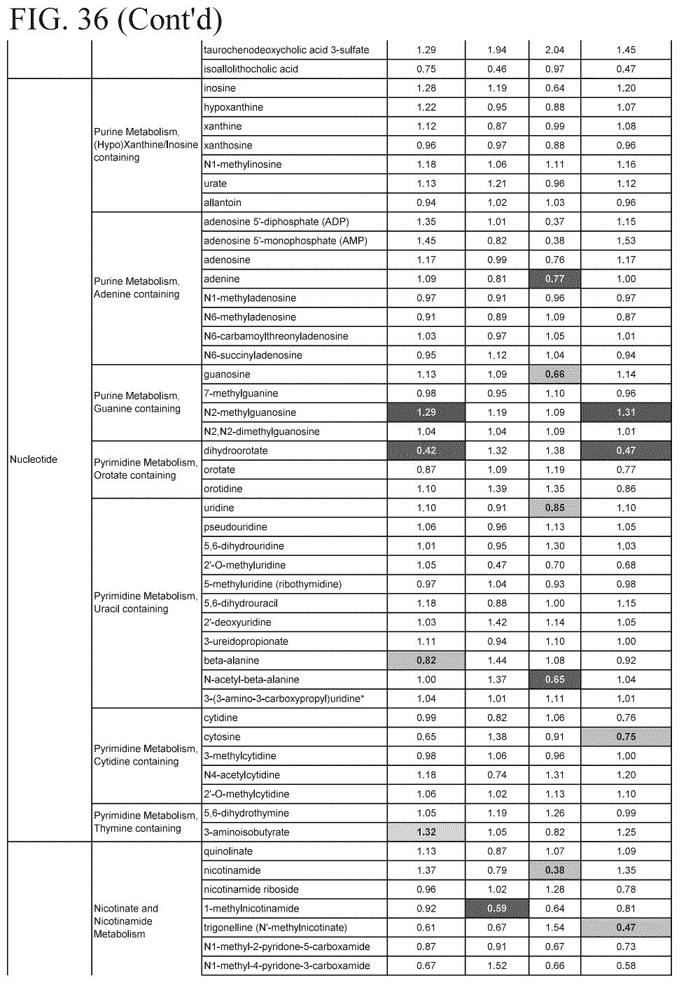

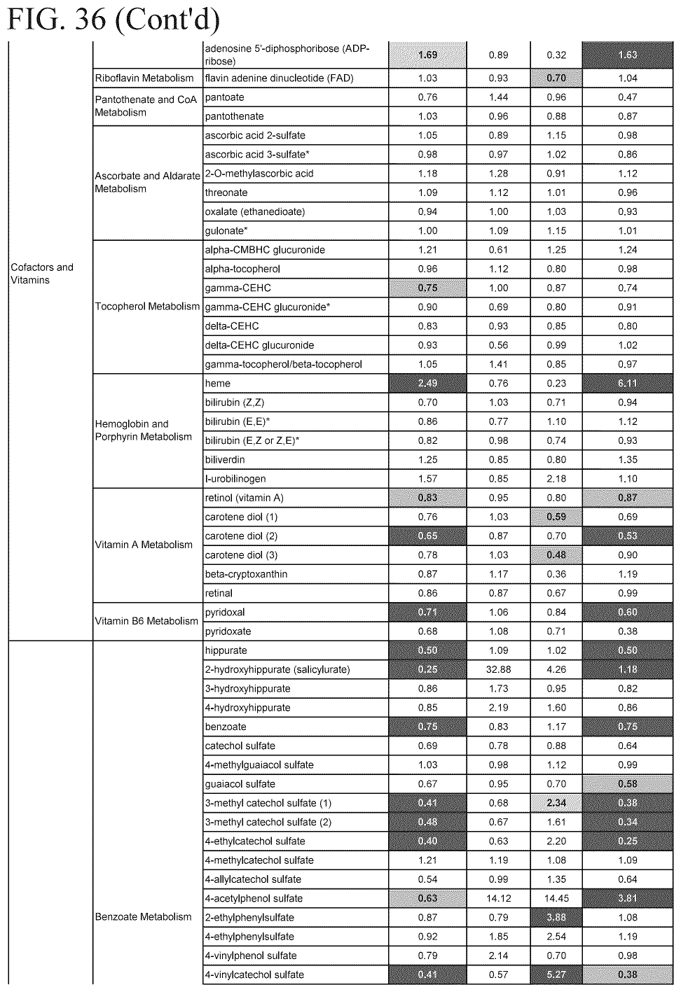

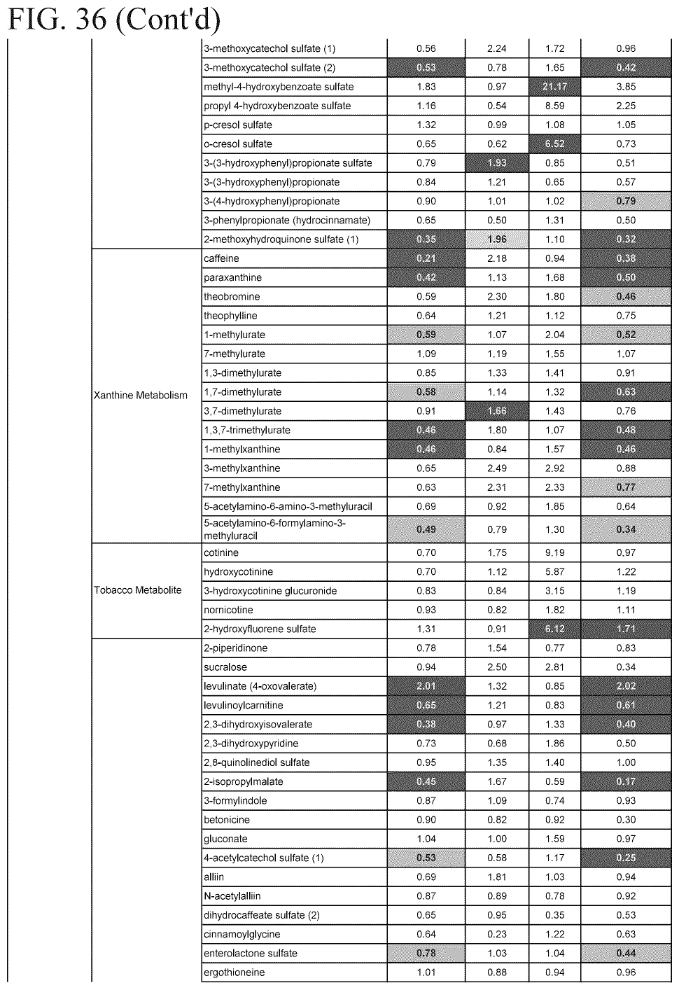

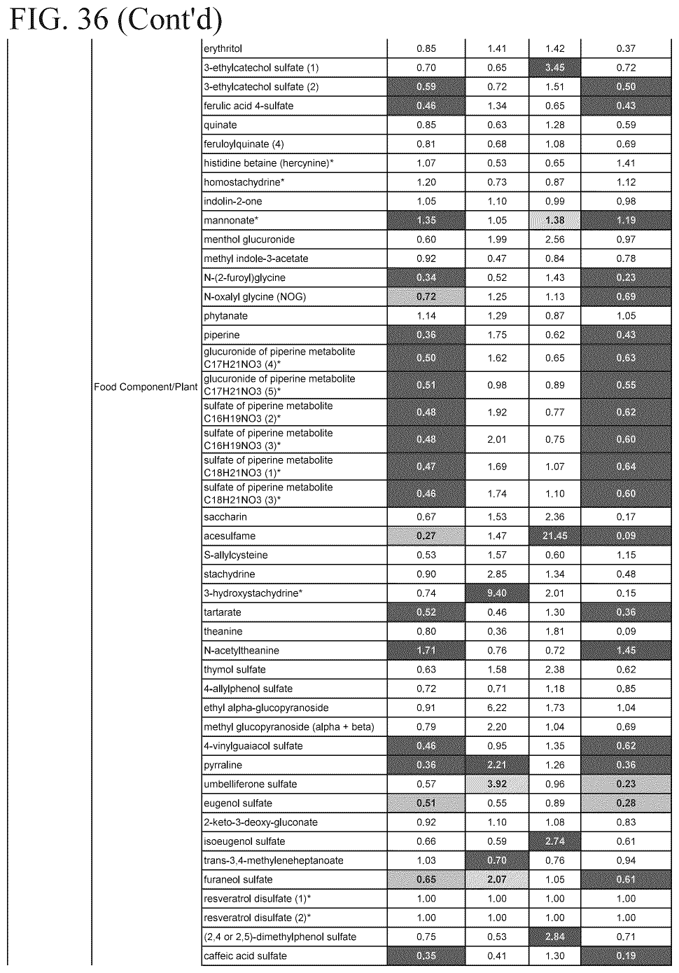

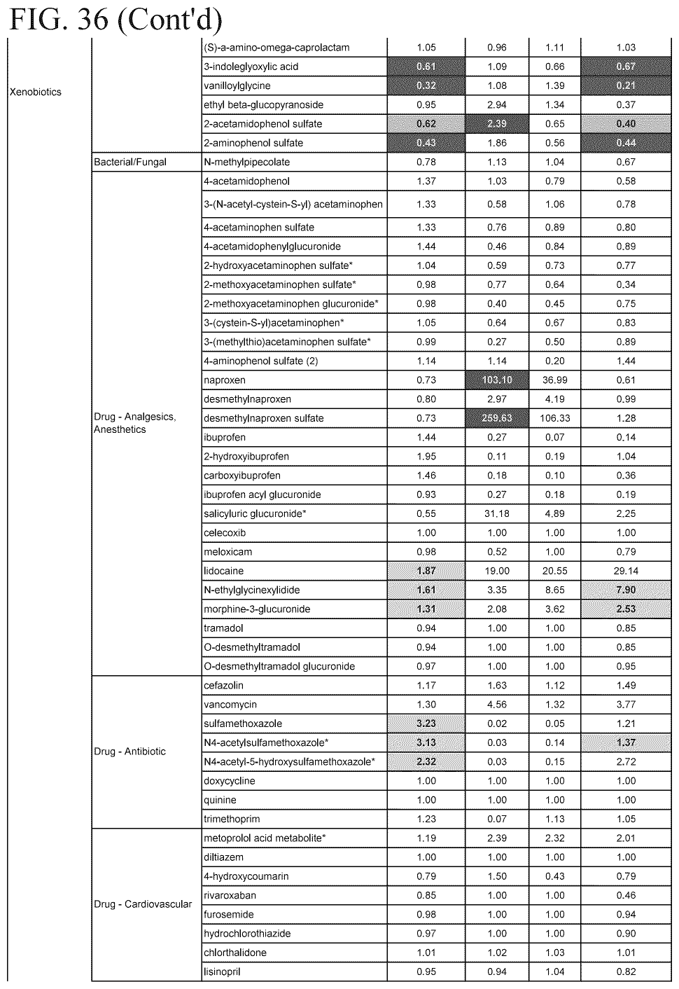

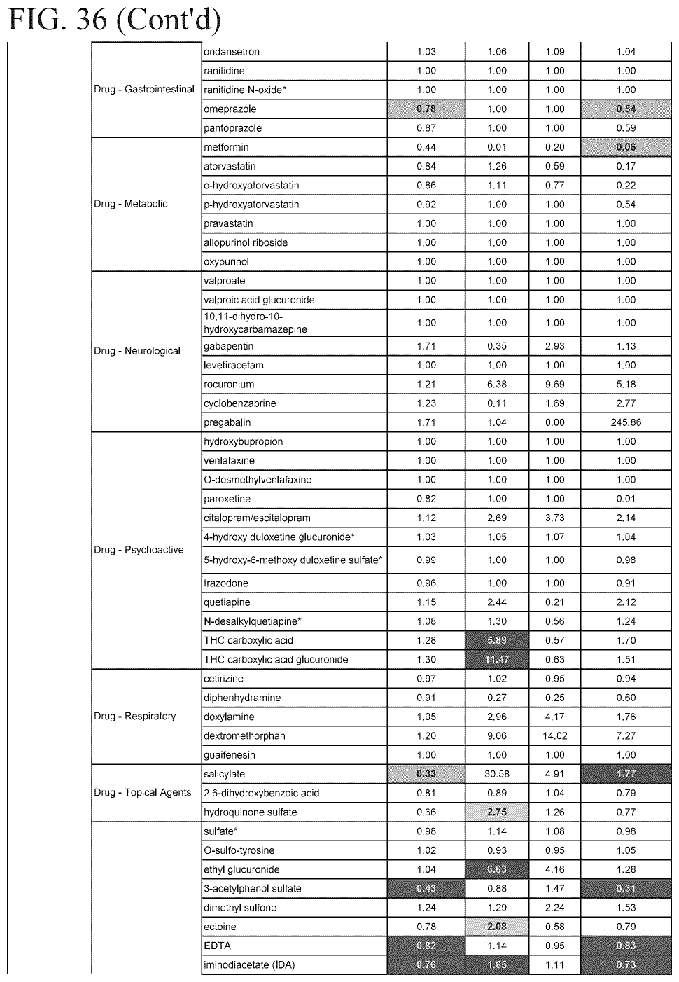

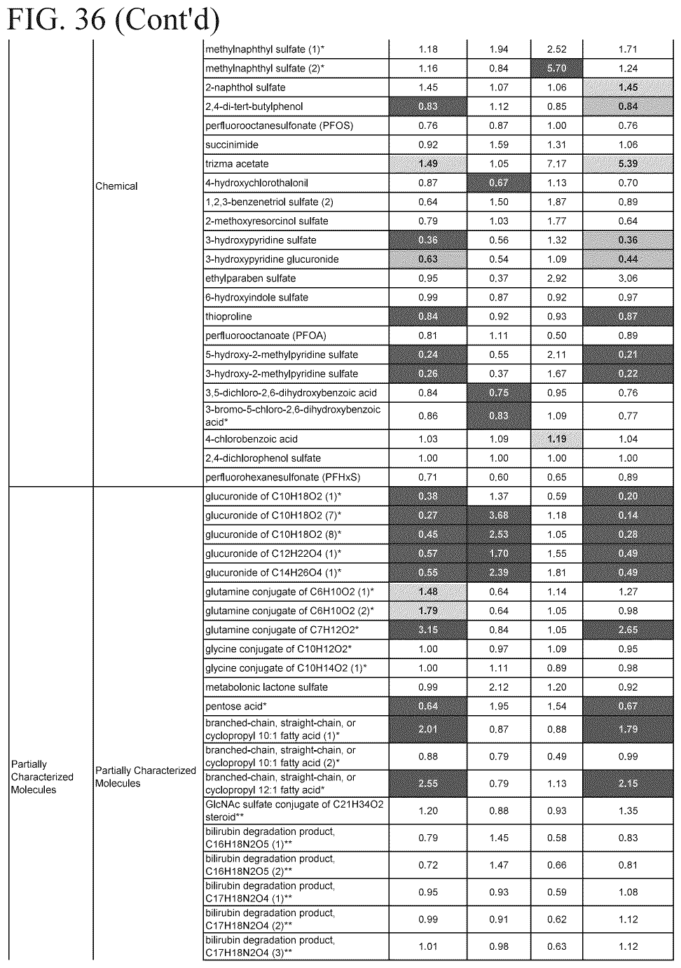

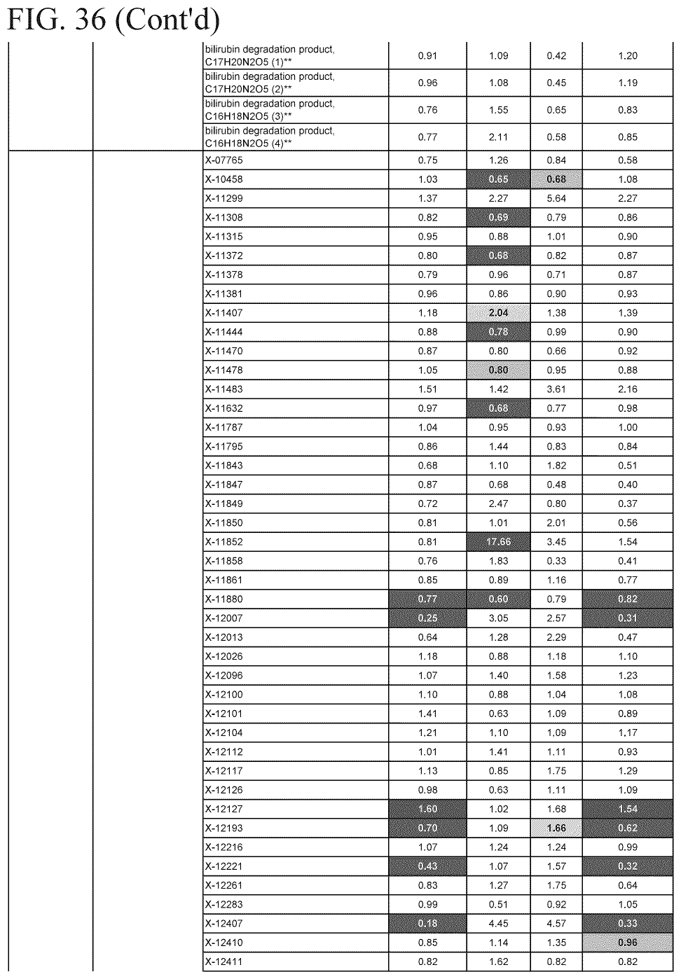

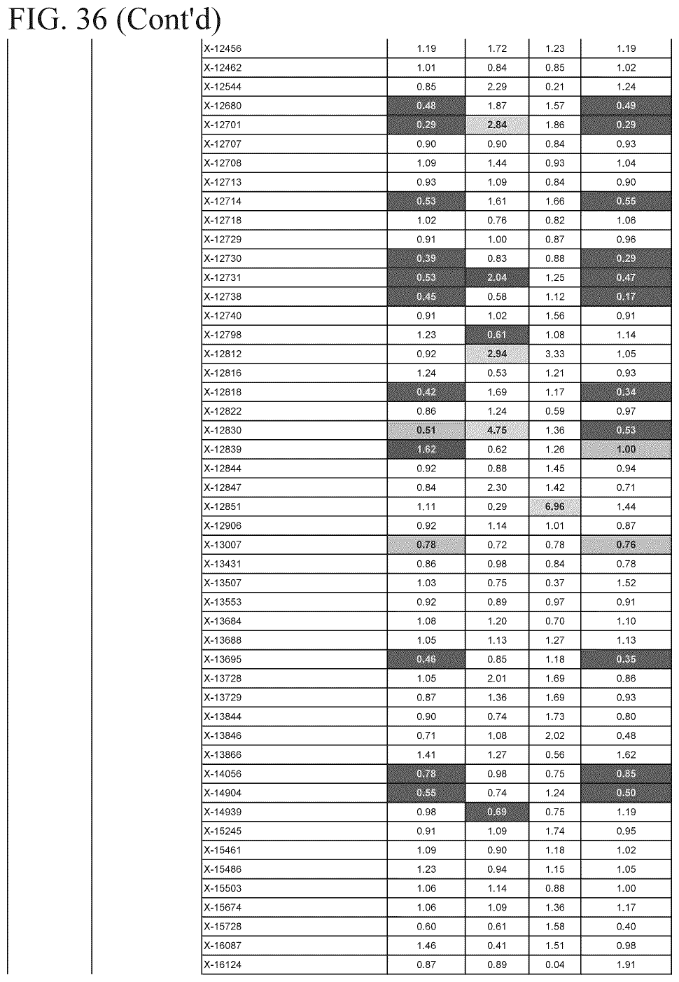

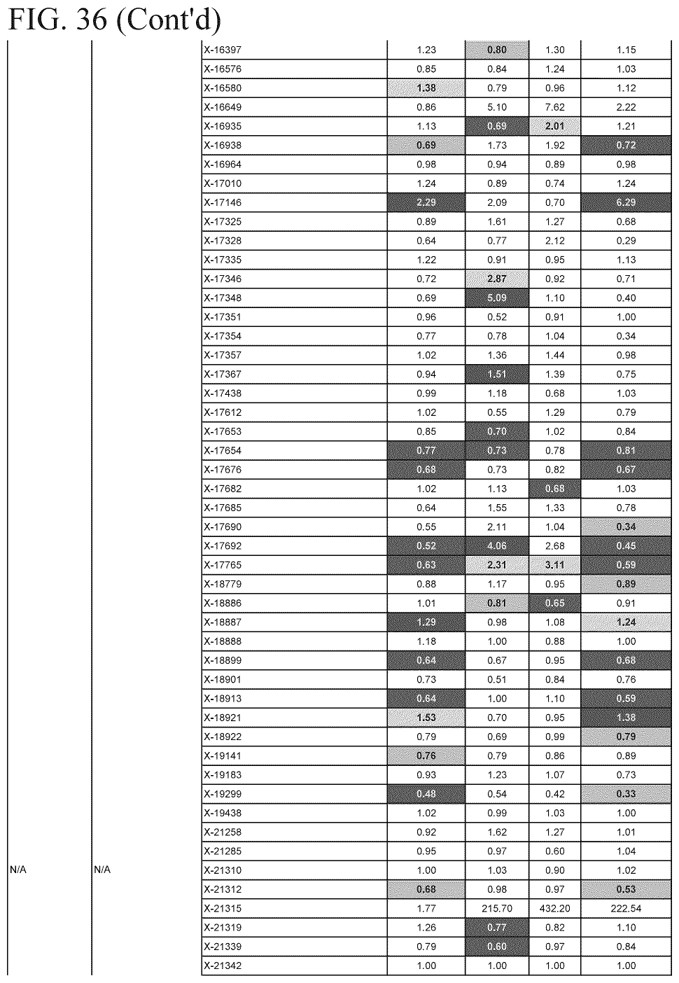

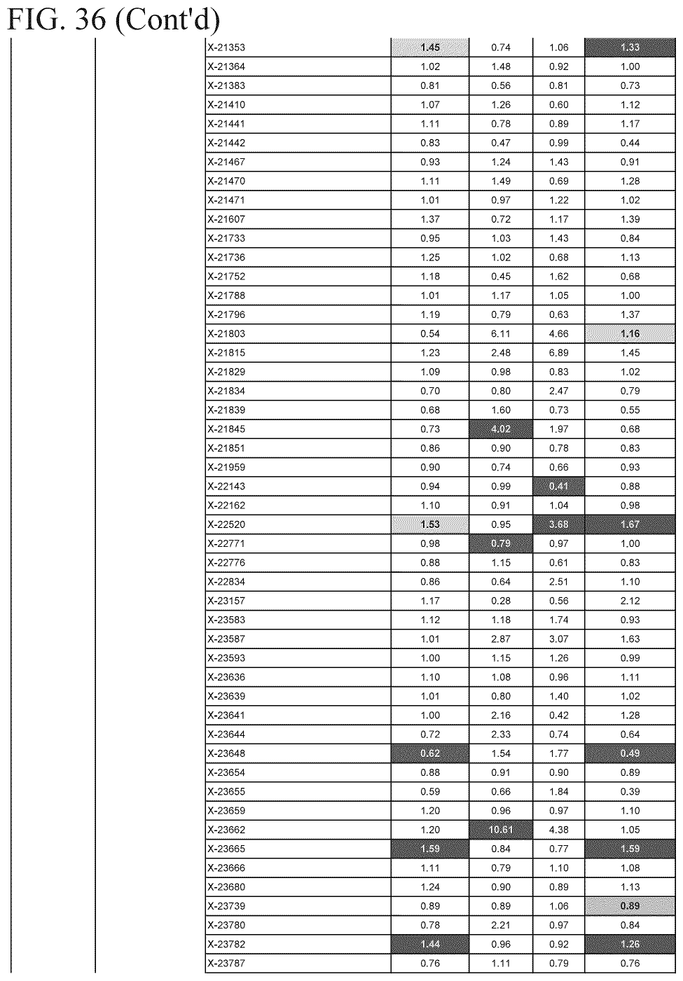

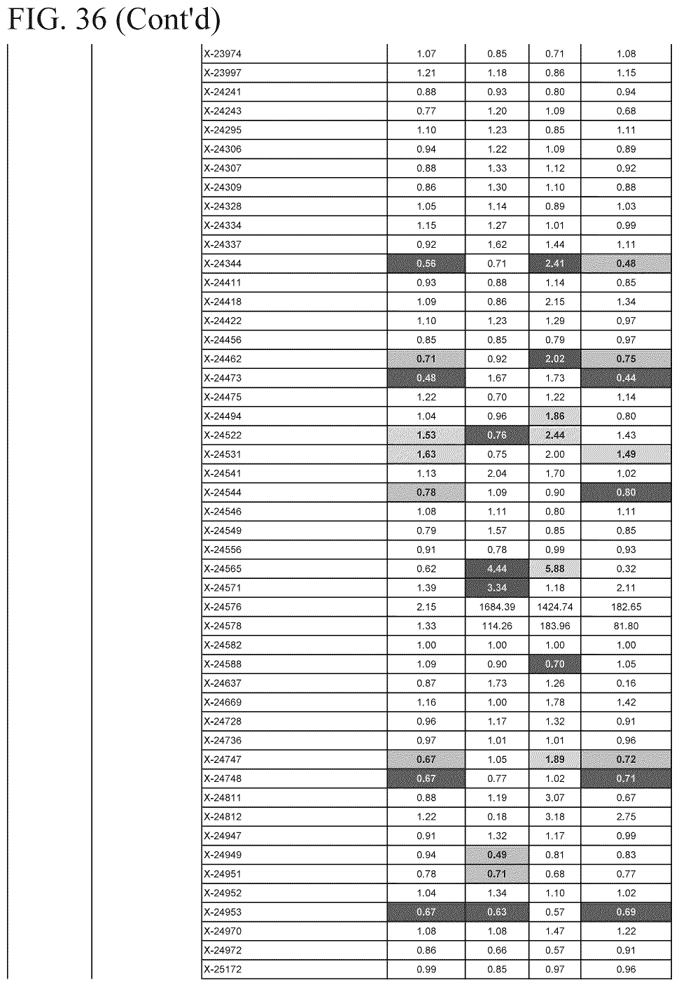

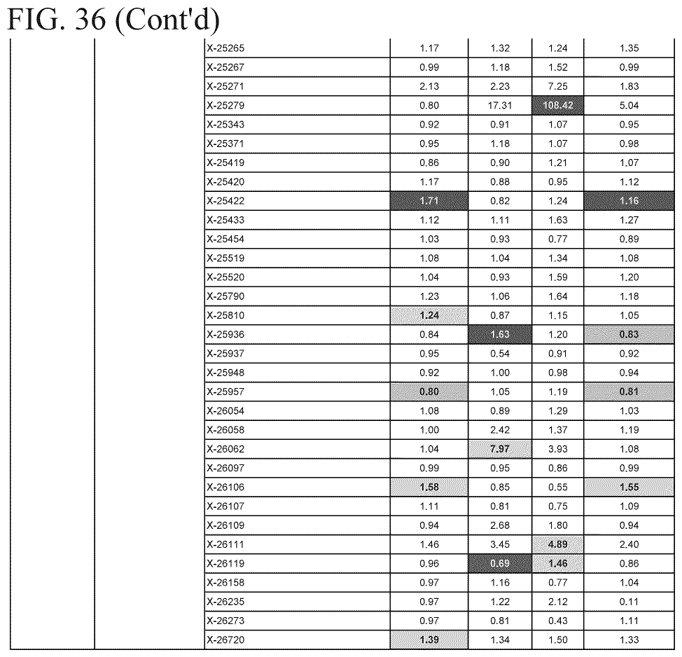

The details of Supplementary Content 2 are shown in FIG. 36.

### Example 4. Comparison of Mass Spectrometry and Fourier Transform Infrared Spectroscopy of Plasma Samples in Identification of Patients with Fracture-Related Infections

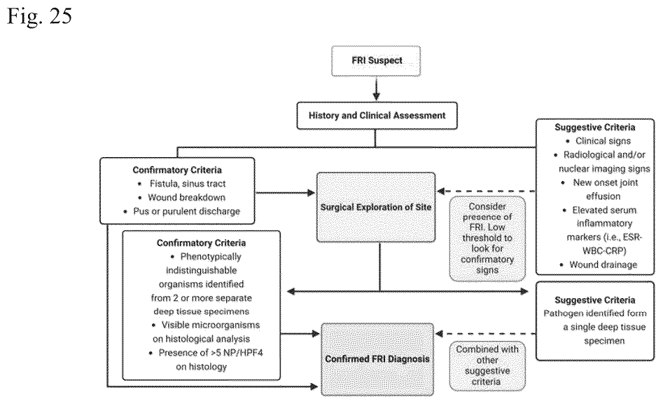

The incidence of postoperative fracture-related infections (FRIs) varies from 5-10%. Depending on the site of fracture, type of infection, and the health care system involved, there is a 1.2 to 6-fold increase in costs associated with treating patients with FRIs worldwide. Diagnosis of FRI remains a challenge due to the historically ill-defined definition of postoperative surgical site infections after fracture repair surgeries, limited investigation of contributing risk factors, and limitations of utilized diagnostic tests [6, 7]. A standardized definition of FRI has only been recently defined and updated [7, 8]. Based on this more widely utilized definition, there are highly specific diagnostic tests for the presence of infection (i.e., confirmatory criteria). However, in this definition, there are also findings that are suggestive of infection in the absence of confirmatory criteria (i.e., suggestive criteria). A diagram of the diagnostic algorithm for suspected FRI cases based on this FRI definition is presented in FIG. 25. Patients with suggestive criteria require further investigation that typically involves more invasive procedures (i.e., multiple deep tissue biopsies for culture and histopathology), which leads to delays in definitive treatment. Considering approximately 25% of patients with suggestive criteria are eventually confirmed as FRI, it is important to improve the sensitivity and specificity of diagnostic tests to facilitate more reliable and timely diagnosis of such cases.

Blood-based biomarkers that have been utilized as suggestive criteria include white blood cell (WBC) count, erythrocyte sedimentation rate (ESR), C-reactive protein (CRP). Recent studies have shown that these three biomarkers are not sufficiently accurate predictors of FRI.

Measurement of other inflammatory biomarkers via enzyme-linked immunosorbent assay (ELISA) methodology has been utilized to evaluate other potential candidate biomarkers in FRI patients. However, in one study that evaluated 49 proteins, only platelet-derived growth factor AB/BB (PDGF-AB/BB), and Monokine induced by gamma interferon (MIG) (in addition to CRP) showed promising results as potential novel candidate diagnostic biomarkers in predicting FRIs. These novel candidate biomarkers still require validation in larger clinical studies. Proteomic analysis of blood samples overcomes the limitation of ELISA-based approaches by simultaneous quantitative measurement of thousands of proteins that can help facilitate the discovery of candidate biomarkers between disease and control samples. A recent study evaluating plasma samples from patients with confirmatory FRI criteria to controls demonstrated systemic activation of the complement and coagulation cascades with significant differences in abundance ratio in 32 out of more than 1000 measured proteins. This approach has been utilized in medical conditions such as sepsis and trauma patients to identify prognostic biomarkers and assess response to treatment. Another utility of proteomics-based approaches in medical research is the ability to evaluate the disease pattern and use multivariate analytical approaches to develop predictive algorithms based on qualitative or quantitative measurements. Fourier-transform infrared (FTIR) spectroscopy of dried films of blood samples is an example of a qualitative pattern recognition approach to disease. Mid-infrared (MIR) absorption of biological samples using FTIR spectroscopy produces unique patterns that are reflective of the sum of all mid-infrared (MIR)-active molecular bonds in a sample. The FTIR spectroscopy of biological fluids (e.g., blood) is a simple, cost-effective methodology that is a clinically accessible tool previously used in diagnosing a variety of disease processes in both animals and humans. The unique spectrum of the sample can then be used as a “fingerprint” since the disease state can alter the molecular composition of biological fluids. A study comparing ELISA-based measurement of proteins versus mid-infrared spectral patterns of plasma samples using FTIR spectroscopy found that both approaches could be used to develop predictive models that performed well in distinguishing between FRI and control samples. The purpose of this study was to compare the performance of mass spectrometry to FTIR spectroscopy in distinguishing between FRI and control plasma samples based on predictive models.

### Materials and Methods

### Patients

This diagnostic, level III study was performed at a single level-one trauma center over a nine month period, from Jun. 25, 2019 to Mar. 24, 2020. Inclusion and exclusion criteria (Table 10) were the same for both the confirmed FRI and control groups. The confirmed FRI group had an additional inclusion criterion of a clinically suspected and subsequently confirmed FRI. No patient in this study had rheumatologic disease or other known chronic inflammatory conditions. Patients who had received antibiotic treatment leading up to their FRI diagnosis were not excluded. All FRI confirmed patients were enrolled prior to surgical intervention for their infection. ESR, CRP, and WBC, as well as three intraoperative cultures and gram stains, were obtained as part of the standard of care for the FRI patients. Patients in the control cohort were identified and matched to the FRI patients based on age (±15 years), time after surgery (±2 weeks), and fracture region. Fracture regions were matched as follows: upper extremity long bones (humerus, radius/ulna, clavicle), lower extremity long bones (femur and tibia), and other lower extremity bones (e.g., patella, ankle, tarsal bones). Control patients were identified through screening clinic schedules for patients undergoing routine fracture care follow-up. All controls had to be infection-free for a minimum of six months after enrollment as determined by routine clinic follow-up, chart review, or phone calls. Written and signed informed consent was obtained from all participating patients prior to enrollment (IRB #1905884760).

TABLE 10
Inclusion and Exclusion Criteria

| Inclusion | Exclusion |
|---|---|
| Age 18 to 85 years inclusive | Hand or Spine fracture |
| Extremity, pelvic ring, or acetabulum | Pregnancy |
| fracture that was surgically treated with | Incarceration |
| retained orthopedic implant within two | Known immunosuppressive state |
| years of blood sample collection | Ongoing treatment with |
| | immunomodulatory drug |
| | Localized or systemic infection |
| | Second or more debridement for infection |
| | Hemodialysis |
| | Venous thromboembolism |
| | Definitive treatment with arthroplasty, K- |
| | wires, or external fixation |

### Blood Collection and Processing

Blood samples were obtained from the FRI cohort preoperatively on the day of surgical intervention to address the infection. Blood samples were obtained from the control cohort during routine fracture care follow-up visits. Specifically, approximately 5 ml of peripheral venous blood was collected in an EDTA purple top tube (BD Vacutainer®, Becton, Dickinson and Company, Franklin Lakes, NJ). The tube was inverted 4-5 times to allow the blood to mix with the anticoagulant before it was centrifuged at 1500 g for 10 minutes. Plasma was then extracted, aliquoted into 500 μL tubes, and stored at −80° C. until batch analysis.

### Mass Spectrometry

Tandem mass tag liquid chromatography-mass spectrometry (TMT LC-MS/MS) was performed by the Center for Proteome Analysis (CPA) at Indiana University School of Medicine. Protocols are described in detail in Becker K, Sharma I, Slaven J E, Mosley A L, Doud E H, Malek S, et al. Proteomic Analyses of Plasma From Patients With Fracture-Related Infection Reveals Systemic Activation of the Complement and Coagulation Cascades. J Orthop Trauma. 2024; 38(3):e111-e9, which is expressly incorporated herein in its entirety.

### FTIR Spectroscopy

Samples were thawed in room temperature (22° C.) and then diluted with potassium thiocyanate (KSCN, SigmaUltra, Sigma-Aldrich Inc, St Louis, MO), as an internal control, in a 2:1 ratio. Using a previously described technique, three 8 μL replicates of each sample were applied on a 96-welled silicon microplate (Bruker Scientific, LLC, Billerica, MA, USA) and allowed to dry at room temperature (20-22° C.) for a minimum of two hours before acquiring the spectra. Each microplate was placed in the multi-sampler (HTS-XT, Bruker Scientific, LLC, Billerica, MA, USA) attachment of an FTIR spectrometer (INVENIO S, Bruker Scientific, LLC, Billerica, MA, USA). The spectra acquisition was performed within 24 hours of the samples being loaded on each microplate. The MIR absorbance spectra in the wavenumber range of 400 to 4,000 cm⁻¹ were recorded using the OPUS software (version 6.5, Bruker Optics, GmbH, Ettlingen, Germany). For each sample evaluation, 512 interferograms were signal averaged and Fourier transformed to produce a nominal resolution of 4 cm⁻¹ for the resulting spectrum. The background spectrum was measured once per plate based on a single empty well in the same location on every plate.

### FTIR Data Preprocessing

Chemometric analysis of the FTIR data was conducted to categorize samples based on their relationship with the health status of the subjects in the study. Furthermore, the analysis aimed to identify the characteristic features of the samples that define their composition and ultimately determine their classification outcome.

The raw data underwent preprocessing steps, which included normalization to the area under the curve, followed by normalization to the KSCN peak using the additive log-ratio method. To remove the baseline drift and noise from the data, the Savitzky-Golay filter was applied, and the KSCN peak was subsequently removed. Further, the discrete cosine transform (DCT) was utilized, which converted the data into the frequency domain.

The DCT is a mathematical transformation that can convert a finite sequence of data points into a combination of cosine functions with varying frequencies. It is primarily used in image and signal processing applications, where it is vital for tasks such as image compression and feature extraction. Therefore, the spectral data was represented as follows:

$${X}_{k} = \sqrt{\frac{2}{N}} \cdot {C}_{k} {\sum }_{n = 0}^{N - 1} {x}_{n} \cdot cos ( \frac{π}{N} ( n + \frac{1}{2} ) k ) for k = 0 , 1 , … , N - 1$$

where Xₖ represents the transformed coefficient at frequency k, and xₙ is the original data sequence. The parameter N denotes the length of the data sequence.

The term \sqrt{2/N} is a normalization factor that ensures the transformation is orthonormal, ensuring that the energy of the original signal x is preserved in the transformed sequence X. The term Cₖ represents another coefficient that depends on the value of k. For k=0, C₀=1/\sqrt{2}, and for k>0, Cₖ=1. A frequency filtering step was then implemented to remove the low-frequency component, which exhibited near-zero variance.

Subsequently, the data underwent univariate feature filtering. This was done by analyzing the entries in the transformed spectral vector one by one using t-tests and Cohen's d calculation to determine their association with the health class. Only the features that demonstrated a separation of more than d>0.5 were retained for further analysis. The DCT operations were performed using R-language for statistical computing and the library DTT (Discrete Trigonometric Transforms).

### Statistical Analysis

### Classification

The processed DC-transformed spectral vectors were used to train the elastic-net logistic regression model, which had an upper limit of 50 for the number of used coefficients. This upper limit was chosen arbitrarily, but the choice was informed by the diminishing, informative predictive content of the features. The model was optimized to minimize the objective function L defined as:

$$L ( β ) = \frac{1}{N} {\sum }_{i = 1}^{N} [ {y}_{i} log ( p ( {y}_{i} = 1 ) ) + ( 1 - {y}_{i} ) log ( 1 - p ( {y}_{i} = 1 ) ) ] + {λ}_{1} [ \frac{1}{2} ( 1 - α ) {\sum }_{j = 1}^{D} \vert {β}_{j} \vert + \frac{1}{2} α {\sum }_{j = 1}^{D} {β}_{j}^{2} ]$$

where β is the vector of coefficients (β₀, β₁, . . . , β_{D}), p(yᵢ=1) is the predicted probability of the positive class, and λ is the regularization parameter controlling the strength of the elastic net regularization. The parameter α controls the mix between l₁ and l₂ regularization, with α=1 representing LASSO (l₁) and α=0 representing Ridge (l₂) regularization. By incorporating the l₁ and l₂ terms, the approach can achieve feature selection while performing its primary classification function. l₁ term facilitates sparsity by inducing many coefficients to become precisely zero. As a result, l1 regularization tends to drive the coefficients of irrelevant or less important features to zero, effectively removing them from the model. The l2 regularization method does not reduce coefficients to exactly zero; rather, it penalizes larger coefficient values. This approach is beneficial in preventing overfitting since it reduces the coefficients' magnitude, making the model less susceptible to individual data points and noise. Although the l2 term does not directly facilitate feature selection, it stabilizes the model and improves its generalization performance by lessening the influence of less relevant features.

Given the limited number of samples, bootstrap-based estimation of the classifier's performance rather was opted for rather than the more commonly used k-fold cross-validation method. To accommodate the small number of examples, 10 bootstrap resampling iterations were performed, with each sample drawn at a size of 100. The model's performance was evaluated by calculating the mean area under the receiver operating characteristic curve (AUROC) and its standard deviation (SD). Each training session was repeated independently repeated 100×, varying the random seeds that determined the bootstrap splits. The final classification result was reported through a meta-analysis of all individual training sessions, assuming a fixed effect model.

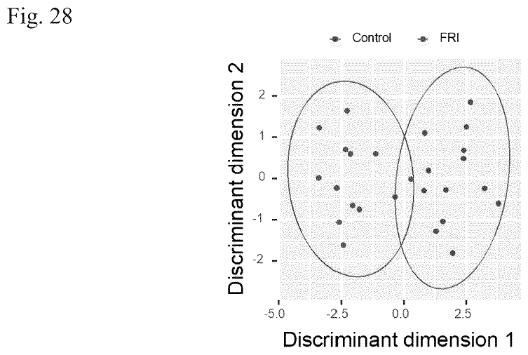

To visualize the classification capability of the selected frequencies, a linear discriminant function was trained with the frequencies selected by the embedded feature selection of the elastic-net regressor. This provided insight into the potential of resolving the two classes using a linear model. The classification performance of the system was expressed as AUROC, Sensitivity, and Specificity with respective 95% confidence intervals (CI₉₅). Although the classification is performed using elastic net regression, the results were visualized by using the subset of selected variables, compressing the information with SVD/PCA, and employing the first 15 principal components (explaining ˜99% of the variance) to build a linear discriminant coordinate space (See FIG. 28).

The analysis pipeline for the mass spectrometry (MS) data differed from that of the MIR data in that the Savitsky-Golay filtering and DCT with subsequent frequency-domain filtering step were not utilized due to the lack of formation of continuous, highly correlated spectra by the MS data. However, the remaining steps of the analysis pipeline were consistent with those employed in the MIR data analysis. Specifically, an elastic net system with embedded feature selection was employed to train the discrimination system and generate a list of the most predictive proteins in a multivariate setting. These proteins were used to perform the overrepresentation analysis employing the Reactome and STRING platforms. The classification and related data processing operations were performed using the caret and glmnet packages in R for statistical analysis computing.

### Evaluation of Associations Between FTIR and MS Data

In order to identify any simple associations between the MS and the MIR readouts, canonical correlation analysis (CCA) and a cross-modal autoencoder (NNE) were used. CCA finds linear projections of the MIR matrix X and the MS matrix Y that are maximally correlated:

$$ρ = {max}_{a , b} \frac{{a}^{T} {\sum }_{XY} b}{\sqrt{( {a}^{T} {\sum }_{XX} a ) ( {b}^{T} {\sum }_{YY} b )}} ,$$

where Σ_{XX}, Σ_{YY} are the within-set covariances and Σ_{XY} the cross-covariance. CCA was applied to the most discriminatory MS proteins and raw FTIR wavenumbers (no DCT) to avoid altering the original spectral features. A cross-modal autoencoder was trained to learn non-linear links between the two modalities. Each input (MIR or MS data) passed through three dense layers and a 2-node bottleneck; the decoder mirrored this path to reconstruct the opposite modality. The network, implemented in TensorFlow/Keras, minimized joint reconstruction error, forcing the bottleneck to capture any shared structure beyond CCA's linear scope.

Eighty-two patients were screened for enrollment, of which 22 confirmed FRIs and 16 controls had samples obtained. Matching, as described above, resulted in 13 pairs. Eight of 13 had at least two positive cultures with phenotypically indistinguishable pathogens obtained during surgery for their infection; all 13 of the FRIs met confirmatory criteria with either fistula/sinus/wound breakdown and/or purulent drainage on initial presentation. Table 11 summarizes patient demographic, clinical, and co-morbidity data for both groups. There were no statistically significant differences in age or fracture region. There was a statistically significant difference in time-points (mean of one week) during the post-operative period at which samples were obtained (P=0.045). Seven patients in the FRI group had received antibiotics within two weeks of their blood draw.

TABLE 11
Description of Cohort.

| | Overall Cohort | FRI | Control | |
|---|---|---|---|---|
| Demographics | (n = 26) | (n = 13) | (n = 13) | P value^{a} |
| Age | 51.3 (14.9) | 51.4 (14.8) | 51.2 (15.5) | 0.953 |
| Sex | | | | 0.226 |
| Male | 16 | 10 | 6 | |
| Female | 10 | 3 | 7 | |
| BMI | 30.2 (7.9) | 28 (4.6) | 32.4 (9.9) | 0.209 |
| Weeks Post Operation | 6.0(4.3) | 6.4 (4.5) | 5.5 (4.4) | 0.043 |
| Clinical | | | | |
| Bone Involvement | | | | >0.999 |
| Femur/Tibia | 18 | 9 | 9 | |
| Patella/Ankle/Foot | 6 | 3 | 3 | |
| Upper Extremity/Clavicle | 2 | 1 | 1 | |
| Implant Used | | | | 0.047 |
| IMN | 12 | 9 | 3 | |
| Plate | 14 | 4 | 10 | |
| Fracture Type | | | | 0.096 |
| Open | 4 | 4 | 0 | |
| Closed | 22 | 9 | 13 | |
| NSAID/Steroid Use | | | | >0.999 |
| Yes | 1 | 1 | 0 | |
| No | 25 | 12 | 13 | |
| Co-morbidities | | | | |
| Diabetes Mellitus | | | | 0.322 |
| Yes | 5 | 1 | 4 | |
| No | 21 | 12 | 9 | |
| History of MRSA | | | | 0.48 |
| Yes | 2 | 2 | 0 | |
| No | 24 | 11 | 13 | |
| Tobacco Use | | | | >0.999 |
| Yes | 7 | 4 | 3 | |
| No | 19 | 9 | 10 | |
| Alcohol Abuse | | | | 0.48 |
| Yes | 2 | 2 | 0 | |
| No | 24 | 11 | 13 | |

Values are means (standard deviation) for continuous data (i.e., age, BMI, weeks post-operation). All other values are counts. BMI (body mass index), IMN (intramedullary nail), NSAID (non-steroidal anti-inflammatory drug), MRSA (multi-drug-resistant Staphylococcus aureus). ^{a}Results from two-sided matched t-test for continuous data and Fisher's Exact test for categorical data. Statistical significance is set at P<0.05.

### FTIR Spectroscopy

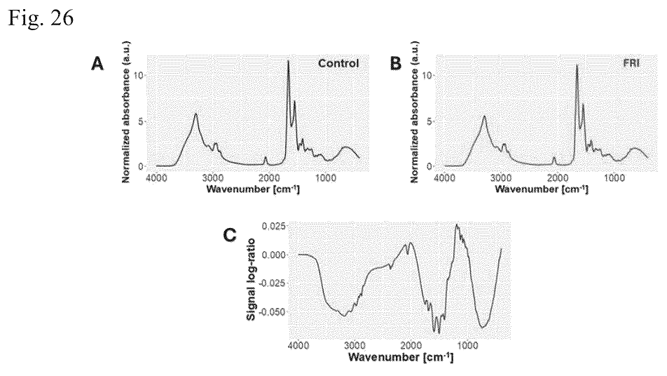

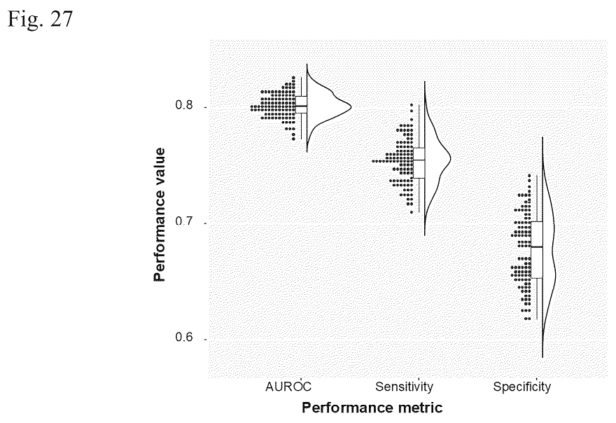

The collected FTIR spectra for the FRI and control groups are presented in FIGS. 26A and 26B, where no clear differences are immediately observable. However, computing the log ratio of the spectra for the two groups reveals distinct variations, as shown in FIG. 26C. The meta-analysis of the multiple bootstrap runs showed that the average AUROC was ≈0.803, CI₉₅ (0.8, 0.81), the average sensitivity was ≈0. 0.755, CI₉₅ (0.75, 0.76), and the specificity was ≈0.677, CI₉₅ (0.672, 0.682). The classification performance of the system is illustrated in FIG. 27 and FIG. 28.

### Mass Spectrometry

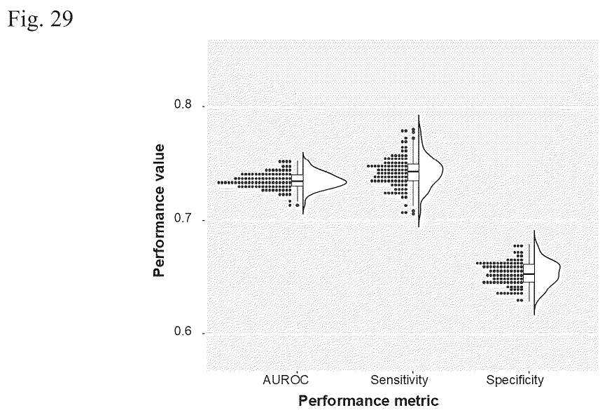

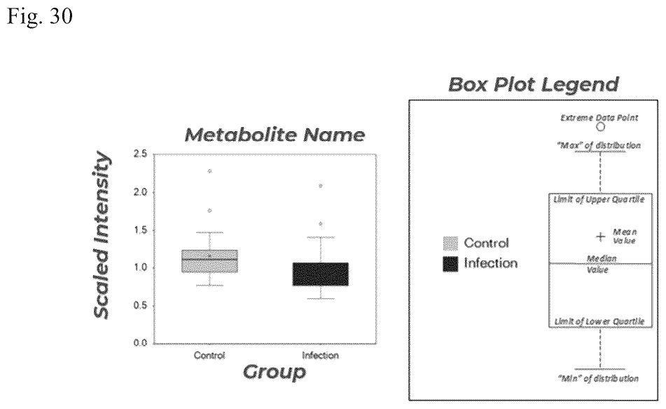

The MS input underwent a data processing procedure comparable to the one previously described. The meta-analysis of the multiple bootstrap runs of the MS-based model showed that the average AUROC was ≈0.735, CI₉₅ (0.732, 0.737), the average sensitivity was ≈0.74, CI₉₅ (0.739, 0.747), and the specificity was ≈0.653, CI₉₅ (0.649, 0.656). The Mass spectrometry-derived model classification performance is illustrated in FIG. 29.

The top 40 features selected by the multiple runs of the sparse model included the proteins listed in Table 11A. These proteins were used to perform the overrepresentation analysis; the detailed results are shown in Table 11A. The pathways identified for these proteins are shown in Table 11B.

TABLE 11A
List of the proteins selected during the multivariate feature selection process.

| Protein | Gene | Annotations |
|---|---|---|
| P02768 | ALB | Serum albumin; Serum albumin, the main protein of plasma, |
| | | has a good binding capacity for water, Ca(2+), Na(+), K(+), |
| | | fatty acids, hormones, bilirubin and drugs. Its main function is |
| | | the regulation of the colloidal osmotic pressure of blood. Major |
| | | zinc transporter in plasma, typically binds about 80% of all |
| | | plasma zinc; Belongs to the ALB/AFP/VDB family |
| P06727 | APOA4 | Apolipoprotein A-IV; May have a role in chylomicrons and |
| | | VLDL secretion and catabolismequired for efficient |
| | | activation of lipoprotein lipase by ApoC-II; potent activator of |
| | | LCAT. Apoa-IV is a major component of HDL and |
| | | chylomicrons; Belongs to the apolipoprotein A1/A4/E family |
| P03952 | KLKB1 | Plasma kallikrein; The enzyme cleaves Lys-Arg and Arg-Ser |
| | | bonds. It activates, in a reciprocal reaction, factor XII after its |
| | | binding to a negatively charged surface. It also releases |
| | | bradykinin from HMW kininogen and may also play a role in |
| | | the renin-angiotensin system by converting prorenin into renin; |
| | | Belongs to the peptidase S1 family. Plasma kallikrein |
| | | subfamily |
| P02766 | TTR | Transthyretin; Thyroid hormone-binding protein. Probably |
| | | transports thyroxine from the bloodstream to the brain; Gla |
| | | domain containing |
| P0C0L5 | C4B | Complement c4b (chido blood group); Complement C4-B; |
| | | Non-enzymatic component of the C3 and C5 convertases and |
| | | thus essential for the propagation of the classical complement |
| | | pathway. Covalently binds to immunoglobulins and immune |
| | | complexes and enhances the solubilization of immune |
| | | aggregates and the clearance of IC through CR1 on |
| | | erythrocytes. C4A isotype is responsible for effective binding |
| | | to form amide bonds with immune aggregates or protein |
| | | antigens, while C4B isotype catalyzes the transacylation of the |
| | | thioester carbonyl group to form ester bonds with carbohydrate |
| | | antigens |
| P04196 | HRG | Histidine-rich glycoprotein; Plasma glycoprotein that binds a |
| | | number of ligands such as heme, heparin, heparan sulfate, |
| | | thrombospondin, plasminogen, and divalent metal ions. Binds |
| | | heparin and heparin/glycosaminoglycans in a zinc-dependent |
| | | manner. Binds heparan sulfate on the surface of liver, lung, |
| | | kidney and heart endothelial cells. Binds to N-sulfated |
| | | polysaccharide chains on the surface of liver endothelial cells. |
| | | Inhibits rosette formation. Acts as an adapter protein and is |
| | | implicated in regulating many processes such as immune |
| | | complex and pathogen clearance, cell chemotaxis, cell [ . . . ] |
| P01042 | KNG1 | Kininogen-1; (1) Kininogens are inhibitors of thiol proteases; |
| | | (2) HMW-kininogen plays an important role in blood |
| | | coagulation by helping to position optimally prekallikrein and |
| | | factor XI next to factor XII; (3) HMW-kininogen inhibits the |
| | | thrombin- and plasmin- induced aggregation of thrombocytes; |
| | | (4) the active peptide bradykinin that is released from HMW- |
| | | kininogen shows a variety of physiological effects: (4A) |
| | | influence in smooth muscle contraction, (4B) induction of |
| | | hypotension, (4C) natriuresis and diuresis, (4D) decrease in |
| | | blood glucose level, (4E) it is a mediator of inflammation [ . . . ] |
| P81605 | DCD | Dermcidin; DCD-1 displays antimicrobial activity thereby |
| | | limiting skin infection by potential pathogens in the first few |
| | | hours after bacterial colonization. Highly effective against |
| | | E. coli, E. faecalis, S. aureus and C. albicans. Optimal pH and salt |
| | | concentration resemble the conditions in sweat. Also exhibits |
| | | proteolytic activity, cleaving on the C-terminal side of Arg and, |
| | | to a lesser extent, Lys residues |
| P05546 | SERPIND1 | Heparin cofactor 2; Thrombin inhibitor activated by the |
| | | glycosaminoglycans, heparin or dermatan sulfate. In the |
| | | presence of the latter, HC-II becomes the predominant |
| | | thrombin inhibitor in place of antithrombin III (AT-III). Also |
| | | inhibits chymotrypsin, but in a glycosaminoglycan- |
| | | independent manner; Belongs to the serpin family |
| Q8NCM8 | DYNC2H1 | Cytoplasmic dynein 2 heavy chain 1; May function as a motor |
| | | for intraflagellar retrograde transport. Functions in cilia |
| | | biogenesis. May play a role in transport between endoplasmic |
| | | reticulum and Golgi or organization of the Golgi in cells (By |
| | | similarity); Dyneins, cytoplasmic |
| Q86YZ3 | HRNR | Hornerin; Component of the epidermal cornified cell |
| | | envelopes; Belongs to the S100-fused protein family |
| Q9NSB4 | KRT82 | Keratin, type II cuticular Hb2; Keratins, type II |
| P29622 | SERPINA4 | Kallistatin; Inhibits human amidolytic and kininogenase |
| | | activities of tissue kallikrein. Inhibition is achieved by |
| | | formation of an equimolar, heat- and SDS-stable complex |
| | | between the inhibitor and the enzyme, and generation of a |
| | | small C-terminal fragment of the inhibitor due to cleavage at |
| | | the reactive site by tissue kallikrein; Belongs to the serpin |
| | | family |
| P01023 | A2M | Alpha-2-macroglobulin; Is able to inhibit all four classes of |
| | | proteinases by a unique ‘trapping’ mechanism. This protein has |
| | | a peptide stretch, called the ‘bait region’ which contains specific |
| | | cleavage sites for different proteinases. When a proteinase |
| | | cleaves the bait region, a conformational change is induced in |
| | | the protein which traps the proteinase. The entrapped enzyme |
| | | remains active against low molecular weight substrates |
| | | (activity against high molecular weight substrates is greatly |
| | | reduced). Following cleavage in the bait region, a thioester |
| | | bond is hydrolyzed and mediates the c [ . . . ] |
| Q6UXB8 | PI16 | Peptidase inhibitor 16; May inhibit cardiomyocyte growth; |
| | | CAP superfamily |
| O43866 | CD5L | CD5 antigen-like; Secreted protein that acts as a key regulator |
| | | of lipid synthesis: mainly expressed by macrophages in |
| | | lymphoid and inflammed tissues and regulates mechanisms in |
| | | inflammatory responses, such as infection or atherosclerosis. |
| | | Able to inhibit lipid droplet size in adipocytes. Following |
| | | incorporation into mature adipocytes via CD36-mediated |
| | | endocytosis, associates with cytosolic FASN, inhibiting fatty |
| | | acid synthase activity and leading to lipolysis, the degradation |
| | | of triacylglycerols into glycerol and free fatty acids (FFA). |
| | | CD5L-induced lipolysis occurs with progression o [ . . . ] |
| P43652 | AFM | Afamin; Vitamin E binding protein. May transport vitamin E |
| | | in body fluids under conditions where the lipoprotein system is |
| | | not sufficient. May be involved in the regulation and transport |
| | | of vitamin E at the blood-brain barrier; Belongs to the |
| | | ALB/AFP/VDB family |
| Q96SB3 | PPP1R9B | Neurabin-2; Seems to act as a scaffold protein in multiple |
| | | signaling pathways. Modulates excitatory synaptic |
| | | transmission and dendritic spine morphology. Binds to actin |
| | | filaments (F-actin) and shows cross-linking activity. Binds |
| | | along the sides of the F-actin. May play an important role in |
| | | linking the actin cytoskeleton to the plasma membrane at the |
| | | synaptic junction. Believed to target protein phosphatase 1/PP1 |
| | | to dendritic spines, which are rich in F-actin, and regulates its |
| | | specificity toward ion channels and other substrates, such as |
| | | AMPA-type and NMDA-type glutamate receptors. Pla [ . . . ] |
| P19823 | ITIH2 | Inter-alpha-trypsin inhibitor heavy chain H2; May act as a |
| | | carrier of hyaluronan in serum or as a binding protein between |
| | | hyaluronan and other matrix protein, including those on cell |
| | | surfaces in tissues to regulate the localization, synthesis and |
| | | degradation of hyaluronan which are essential to cells |
| | | undergoing biological processes; Belongs to the ITIH family |
| P13645 | KRT10 | Keratin, type I cytoskeletal 10; Keratins, type I |
| O76096 | CST7 | Cystatin-F; Inhibits papain and cathepsin L but with affinities |
| | | lower than other cystatins. May play a role in immune |
| | | regulation through inhibition of a unique target in the |
| | | hematopoietic system; Cystatins, type 2 |
| Q96PD5 | PGLYRP2 | N-acetylmuramoyl-L-alanine amidase; May play a scavenger |
| | | role by digesting biologically active peptidoglycan (PGN) into |
| | | biologically inactive fragments. Has no direct bacteriolytic |
| | | activity; Belongs to the N-acetylmuramoyl-L-alanine amidase |
| | | 2 family |
| P02751 | FN1 | Fibronectin 1; Fibronectin type III domain containing; |
| | | Endogenous ligands |
| Q5SYB0 | FRMPD1 | FERM and PDZ domain-containing protein 1; Stabilizes |
| | | membrane-bound GPSM1, and thereby promotes its |
| | | interaction with GNAI1; FERM domain containing |
| P55103 | INHBC | Inhibin beta C chain; Inhibins and activins inhibit and activate, |
| | | respectively, the secretion of follitropin by the pituitary gland. |
| | | Inhibins/activins are involved in regulating a number of diverse |
| | | functions such as hypothalamic and pituitary hormone |
| | | secretion, gonadal hormone secretion, germ cell development |
| | | and maturation, erythroid differentiation, insulin secretion, |
| | | nerve cell survival, embryonic axial development or bone |
| | | growth, depending on their subunit composition. Inhibins |
| | | appear to oppose the functions of activins; Belongs to the TGF- |
| | | beta family |
| Q9Y5Y7 | LYVE1 | Lymphatic vessel endothelial hyaluronic acid receptor 1; |
| | | Ligand-specific transporter trafficking between intracellular |
| | | organelles (TGN) and the plasma membrane. Plays a role in |
| | | autocrine regulation of cell growth mediated by growth |
| | | regulators containing cell surface retention sequence binding |
| | | (CRS). May act as a hyaluronan (HA) transporter, either |
| | | mediating its uptake for catabolismwithin lymphatic |
| | | endothelial cells themselves, or its transport into the lumen of |
| | | afferent lymphatic vessels for subsequent re-uptake and |
| | | degradation in lymph nodes |
| P04278 | SHBG | Sex hormone-binding globulin; Functions as an androgen |
| | | transport protein but may also be involved in receptor mediated |
| | | processes. Each dimer binds one molecule of steroid. Specific |
| | | for 5-alpha-dihydrotestosterone, testosterone, and 17-beta- |
| | | estradiol. Regulates the plasma metabolic clearance rate of |
| | | steroid hormones by controlling their plasma concentration |
| P02760 | AMBP | Alpha-1-microglobulin/bikunin precursor; Protein AMBP; |
| | | Inter-alpha-trypsin inhibitor inhibits trypsin, plasmin, and |
| | | lysosomal granulocytic elastase. Inhibits calcium oxalate |
| | | crystallization; Lipocalins |
| P02765 | AHSG | Alpha-2-HS-glycoprotein; Promotes endocytosis, possesses |
| | | opsonic properties and influences the mineral phase of bone. |
| | | Shows affinity for calcium and barium ions; Cystatins, type 4 |
| Q6UY14 | ADAMTSL4 | ADAMTS-like protein 4; Positive regulation of apoptosis. May |
| | | facilitate FBN1 microfibril biogenesis; ADAMTS like |
| Q8NCN4 | RNF169 | E3 ubiquitin-protein ligase RNF169; Probable E3 ubiquitin- |
| | | protein ligase that acts as a negative regulator of double-strand |
| | | breaks (DSBs) repair following DNA damage. Recruited to |
| | | DSB repair sites by recognizing and binding ubiquitin |
| | | catalyzed by RNF168 and competes with TP53BP1 and |
| | | BRCA1 for association with RNF168-modified chromatin, |
| | | thereby acting as a negative regulator of DSBs repair. E3 |
| | | ubiquitin- protein ligase activity is not required for regulation |
| | | of DSBs repair; Ring finger proteins |
| P30492 | HLA-B | HLA class I histocompatibility antigen, B-7 alpha chain; |
| | | Involved in the presentation of foreign antigens to the immune |
| | | system; C1-set domain containing |
| O14978 | ZNF263 | Zinc finger protein 263; Might play an important role in basic |
| | | cellular processes as a transcriptional repressor; Belongs to the |
| | | krueppel C2H2-type zinc-finger protein family |
| P02647 | APOA1 | Apolipoprotein A-I; Participates in the reverse transport of |
| | | cholesterol from tissues to the liver for excretion by promoting |
| | | cholesterol efflux from tissues and by acting as a cofactor for |
| | | the lecithin cholesterol acyltransferase (LCAT). As part of the |
| | | SPAP complex, activates spermatozoa motility; |
| | | Apolipoproteins |
| Q15849 | SLC14A2 | Urea transporter 2; Specialized low-affinity vasopressin- |
| | | regulated urea transporter. Mediates rapid transepithelial urea |
| | | transport across the inner medullary collecting duct and plays |
| | | a major role in the urinary concentrating mechanism; Solute |
| | | carriers |
| P06396 | GSN | Gelsolin; Calcium-regulated, actin-modulating protein that |
| | | binds to the plus (or barbed) ends of actin monomers or |
| | | filaments, preventing monomer exchange (end-blocking or |
| | | capping). It can promote the assembly of monomers into |
| | | filaments (nucleation) as well as sever filaments already |
| | | formed. Plays a role in ciliogenesis; Gelsolin/villins |
| P05452 | CLEC3B | Tetranectin; Tetranectin binds to plasminogen and to isolated |
| | | kringle 4. May be involved in the packaging of molecules |
| | | destined for exocytosis; C-type lectin domain containing |
| P15090 | FABP4 | Fatty acid-binding protein, adipocyte; Lipid transport protein |
| | | in adipocytes. Binds both long chain fatty acids and retinoic |
| | | acid. Delivers long-chain fatty acids and retinoic acid to their |
| | | cognate receptors in the nucleus (By similarity); Belongs to the |
| | | calycin superfamily. Fatty-acid binding protein (FABP) family |
| P00748 | F12 | Coagulation factor xii (hageman factor); Coagulation factor |
| | | XII; Factor XII is a serum glycoprotein that participates in the |
| | | initiation of blood coagulation, fibrinolysis, and the generation |
| | | of bradykinin and angiotensin. Prekallikrein is cleaved by |
| | | factor XII to form kallikrein, which then cleaves factor XII first |
| | | to alpha-factor XIIa and then trypsin cleaves it to beta- factor |
| | | XIIa. Alpha-factor XIIa activates factor XI to factor XIa |

TABLE 11B
Pathways identified by Reactome knowledgebase for proteins in Table 11A.

| Pathway ID | Description | Identified proteins |
|---|---|---|
| R-HSA-114608 | Platelet degranulation | P01023, P01042, P02647, P02751, |
| | | P02765, P02768, P04196, P05452, |
| | | P29622 |
| R-HSA-76005 | Response to elevated platelet | P01023, P01042, P02647, P02751, |
| | cytosolic Ca2+ | P02765, P02768, P04196, P05452, |
| | | P29622 |
| R-HSA-140837 | Intrinsic Pathway of Fibrin Clot | P00748, P01023, P01042, P03952, |
| | Formation | P05546 |
| R-HSA-8957275 | Post-translational protein | P01042, P02647, P02751, P02765, |
| | phosphorylation | P02768, P05546, P19823 |
| R-HSA-381426 | Regulation of Insulin-like | P01042, P02647, P02751, P02765, |
| | Growth Factor (IGF) transport | P02768, P05546, P19823 |
| | and uptake by Insulin-like | |
| | Growth Factor Binding Proteins | |
| | (IGFBPs) | |
| R-HSA-76002 | Platelet activation, signaling and | P01023, P01042, P02647, P02751, |
| | aggregation | P02765, P02768, P04196, P05452, |
| | | P29622 |
| R-HSA-109582 | Hemostasis | P00748, P01023, P01042, P01871, |
| | | P02647, P02751, P02765, P02768, |
| | | P03952, P04196, P05452, P05546, |
| | | P29622 |
| R-HSA-140877 | Formation of Fibrin Clot | P00748, P01023, P01042, P03952, |
| | (Clotting Cascade) | P05546 |
| R-HSA-8963898 | Plasma lipoprotein assembly | P01023, P02647, P06727 |
| R-HSA-977225 | Amyloid fiber formation | P02647, P02766, P06396, P06727 |
| R-HSA-174824 | Plasma lipoprotein assembly, | P01023, P02647, P02768, P06727 |
| | remodeling, and clearance | |
| R-HSA-8963899 | Plasma lipoprotein remodeling | P02647, P02768, P06727 |
| R-HSA-8963888 | Chylomicron assembly | P02647, P06727 |
| R-HSA-8963901 | Chylomicron remodeling | P02647, P06727 |
| R-HSA-8963896 | HDL assembly | P01023, P02647 |
| R-HSA-975634 | Retinoid metabolism and | P02647, P02766, P06727 |
| | transport | |
| R-HSA-6806667 | Metabolism of fat-soluble | P02647, P02766, P06727 |
| | vitamins | |
| R-HSA-8964058 | HDL remodeling | P02647, P02768 |
| R-HSA-2168880 | Scavenging of heme from plasma | P02647, P02760, P02768 |
| R-HSA-1474228 | Degradation of the extracellular | P01023, P02751, P03952 |
| | matrix | |
| R-HSA-6798695 | Neutrophil degranulation | P02765, P02766, P06396, P30492, |
| | | Q86YZ3 |

Both analyses, the linear CCA and the non-linear cross-modal auto-encoder, failed to reveal a straightforward link between the MIR spectra and the MS proteome. The canonical correlations never exceeded permutation-based significance thresholds, and although the auto-encoder achieved a low reconstruction loss, its two-node bottleneck did not separate FRI samples from controls.

The predictive potential of FTIR spectral data may be well-established; however, its limited interpretability in the context of proteomics findings presents a considerable challenge. This issue primarily stems from the inherent complexity of the samples' spectra, which prevents the unique attribution of individual spectral peaks to specific proteins or relevant biomarkers. Furthermore, to enhance the informational content and improve the robustness of predictive models, the spectral data undergoes additional compression through the application of the DCT. While this transformation optimizes the data for classification purposes, it further diminishes its interpretability relative to the original spectral input. It is crucial to underscore that the FTIR spectral features identified through the elastic net approach do not correspond to readily interpretable wavelengths, and currently, no straightforward method exists to restore such interpretability.

The elastic net classifier's embedded feature selection capability also effectively worked with MS data input and yielded a selection of proteins that jointly contributed to the distinguishability of the two experimental cohorts. Nevertheless, it is important to acknowledge that the overall accuracy achieved through proteomics was surprisingly lower than that of the FTIR spectra obtained from FTIR spectroscopy (80% versus 73%, respectively). Based on this observation, two conclusions can be drawn. Firstly, it should be noted that diagnostic methods that are explainable, such as proteomics, may not always exhibit the best level of performance. Secondly, the results provide significant motivation to research further phenotypic biophysical diagnostic techniques, such as MIR, which already surpass certain complex “omic” analyses due to their high prediction and cost-effectiveness.

The discrepancy between the predictive models of FTIR spectroscopy and mass spectrometry can be further explained by the differences in what is detected on each platform. Proteomic analysis using mass spectrometry can only detect proteins, and even then, it has limitations in identifying proteins that may be low in abundance but crucial to the pathology under investigation, along with technical challenges that can result in inaccurate conclusions. The FTIR spectroscopy approach, on the other hand, provides a spectrum that is representative of all analytes within the sample that have MIR active molecular bonds. Therefore, the spectral “fingerprint” is not solely representative of the protein components of the samples but also lipids, sugars, and deoxyribonucleic acid (DNA). This is also the disadvantage of using FTIR spectroscopy, as it cannot be used to quantify compounds within a complex biological fluid (e.g., plasma) due to the overlap of the spectra of individual molecules within the sample. The unmixing approaches are available, but in the absence of pure compound controls, one is limited to blind unmixing heuristics, which may not necessarily guarantee robust, reproducible, or correct results. Therefore, FTIR spectroscopy should be considered as a complementary analytical approach to other quantitative instruments when the goal is the discovery of new biomarkers and/or putative therapeutic targets. The fact that FTIR spectroscopy performs better than mass spectrometry in this study may be attributed to the influence of non-protein metabolites that are contributing to the observed differences (e.g., the metabolome). This can also be considered as an advantage.

A follow-up study is certainly warranted. Firstly, the FTIR spectra may be further validated in larger sample sets as a purely screening diagnostic biomarker without providing information on why the differences are observed. Secondly, the model performance based on the FTIR spectra can be compared to other quantitative techniques (e.g., multi-omics) to determine what molecular components are contributing to the differences between the disease state and controls. The integration of information from various omics data types (i.e., genomics, epigenomics, transcriptomics, proteomics, lipidomics, and metabolomics) can provide a more global mapping of a disease state. This global understanding of a disease state can aid in furthering knowledge of disease pathophysiology and provide targets for therapeutic interventions.

Conversely, the MS data enables the identification of proteins that account for the observed differences facilitating the interpretation of their roles in various cellular and molecular pathways. Following a multivariate feature selection process, certain proteins were determined to be particularly predictive. The overrepresentation analysis of these proteins indicated the activation of expected pathways, including increased platelet cytosolic Ca²⁺, platelet activation, signaling and aggregation, platelet degranulation, hemostasis, and neutrophil degranulation.

Interestingly, despite the strong individual performance of MS and mid-infrared FTIR spectroscopy in distinguishing FRI cases from controls, neither CCA analysis nor the cross-modal auto-encoder revealed a statistically significant link between the two modalities. Several (not mutually exclusive) mechanisms could explain this outcome. The first is simply power: with only 26 paired samples, high-dimensional noise can swamp any genuine cross-signal, whether linear or non-linear. A second possibility is biological complementarity: FTIR records a composite vibrational fingerprint of every chemical species in the specimen, whereas MS measures the relative abundance of specific proteins. Each platform may therefore be capturing different layers of the same pathological cascade, yielding complementary predictive cues. Conversely, the modalities might encode redundant biology in ways that remain uncorrelated because their feature distributions, dynamic ranges, or batch structures differ; latent confounders could drive parallel changes in both data sets without producing observable covariation.

Methodological limits also matter. CCA is restricted to linear projections, and the auto-encoder, constrained to a two-node bottleneck for over-fitting control, may be too small to capture complex, many-to-many mappings. Technical variance unique to each assay adds further unmodelled error. Taken together, these considerations make the current null result inconclusive rather than dispositive: the absence of detectable association does not imply that no mechanistic link exists. Resolving whether the spectroscopic fingerprint and the proteome intersect will require a much larger cohort and integrative models that can handle sparse, non-linear, and higher-dimensional relationships.

The effectiveness of both spectroscopy-based systems in achieving high predictability relies on performing feature selection. High dimensionality in spectral data can make classifiers significantly inefficient if entire, rich datasets are used without careful consideration. The feature selection process enhances classification performed with machine learning algorithms by eliminating irrelevant or redundant features from the dataset. This process is essential since including irrelevant features can adversely affect the accuracy and performance of the classification models. By selecting the most relevant features, the dimensionality of the data is reduced, leading to several benefits, such as reducing measurement costs and storage requirements, coping with limited training sample sets, reducing training and utilization time, and facilitating data visualization and understanding. Feature selection also helps to improve classification accuracy, reduce computation complexity, and enhance the performance of machine-learning models. The feature selection process using the elastic net has been widely applied in various domains, including genetics, image processing, bioengineering, and other fields.

In terms of mechanistic explanations, the interplay between hemostasis pathways and infection, particularly in the context of sepsis and the immune response, is well established. The close interactions between immune defense and hemostasis have been extensively documented in bacterial infections, and hemostasis has been associated with an increased susceptibility to bacterial sepsis. Activation of platelet degranulation-related pathways is an expected finding in areas of infection. Platelets possess the ability to internalize/entrap pathogens and confine them within engulfment vacuoles while also releasing platelet-derived cytokines that bolster the host's defense against viral infections. Moreover, their degranulation process not only directly eliminates pathogens but also modulates the activity of other immune cells, as platelets are capable of influencing the functions and recruitment of neutrophils, endothelium, and lymphocytes to sites of tissue damage or infection. Another important pathway signifying the presence of an inflammatory response to the infection was neutrophil degranulation. Neutrophils serve as the initial line of defense for the body against invading pathogens, particularly bacteria. When they are activated in a suitable manner, they release several pro-inflammatory cytokines and exhibit MHC Class II expression, which enables the presentation of antigen to T cells and triggers their activation [66].

Work-up of FRI is largely based upon history and physical exam, blood tests (i.e., white WBC, ESR, and CRP), radiographs, and occasionally advanced imaging (see FIG. 25). However, WBC, ESR, and CRP have limited predictive value for FRI. Quantitative histology and culture from intra-operative tissue samples are useful tools to diagnose FRI but require invasive testing and are dependent on sample quality. Further, quantitative histology is only validated for chronic or late-onset FRI [68], and culture results are not readily available intra-operatively, leading to delay in definitive treatment. Improved FRI diagnostic strategies are needed to turn clinical scenarios presenting with suggestive criteria into ones with confirmatory criteria to the operating room. The data presented in this study are a step towards improved biomarkers for FRI diagnosis to overcome these limitations. Future studies to validate these methodology in larger population of patients can lead to adaptation of these tests into bed-side diagnostic tests to diagnose FRI non-invasively and follow patients during treatment to determine success would be valuable milestones.

The limitations of the study are the heterogeneous nature of fracture types included, small sample size, and single time-point sampling. However, despite these limitations, these preliminary results provide a baseline for further interrogation of FTIR spectroscopy and MS-based proteomic analysis in larger cohorts to continue the process of validating each technique as a diagnostic tool. If proven effective, the FTIR spectroscopy approach has several advantages over mass spectrometry, which can strengthen its use and transform it into a bedside test. These advantages include low cost, rapidity, simplicity, and adjuvant-free technique with minimal sample preparation requirements for conducting FTIR spectroscopy of biological fluids.

The results of this example demonstrate that FTIR spectroscopy and mass spectrometry-based proteomics are potential diagnostic biomarker candidates for FRI diagnosis. The predictive models based on the FTIR spectra had better performance compared to mass spectrometry protein abundance ratio data, which is likely due to the differences in methodologies and their measurement targets. The results of this study require further investigation and validation in larger cohorts of patients and additional time points of sample collection.

### Example 5. Characterizing the Metabolome in Patients with Confirmed Fracture Related Infection (FRI)

### Objective

### Purpose of Experiment

The goal of this study was to identify biochemical differences in plasma samples derived from fracture patients who go on to heal without and with infection.

### Experimental Procedures

### Experimental Design

Global metabolomic profiles of 54 samples were determined from the experimental groups outlined in the table below.

TABLE 12
The experimental design of this study.

| Group | Description | N |
|---|---|---|
| Control | Plasma from fracture patients | 27 |
| | that healed without infection | |
| Infection | Plasma from fracture patients | 27 |
| | that developed an infection | |

### Results and Biological Interpretation

### Metabolite Summary and Significantly Altered Biochemicals

The present plasma dataset comprises a total of 1,453 biochemicals, 1,182 compounds of known identity (named biochemicals) and 271 compounds of unknown structural identity (unnamed biochemicals). Unnamed biochemicals are reliably detected peaks with the same ion mass and fragmentation patterns. Unlike the named compounds, they have not yet been assigned a specific chemical structure, either due to the lack of purified standard to compare to or from being truly unknown at this time. Following normalization to volume of sample extracted, imputation of missing values, if any, with the minimum observed value for each compound, and log transformation, ANCOVA, Welch's two sample t-Test, and matched pairs t-Test were used to identify biochemicals that differed significantly between experimental groups. A summary of the numbers of biochemicals that achieved statistical significance (p<0.05), as well as those approaching significance (0.05<p<0.10), is shown below.

An estimate of the false discovery rate (q-value) is calculated to take into account the multiple comparisons that normally occur in metabolomic-based studies. For example, when analyzing 200 compounds, it could be expected to see about 10 compounds meeting the p≤0.05 cut-off by random chance. The q-value describes the false discovery rate; a low q-value (q<0.10) is an indication of high confidence in a result. While a higher q-value indicates diminished confidence, it does not necessarily rule out the significance of a result. Other lines of evidence may be taken into consideration when determining whether a result merits further scrutiny. Such evidence may include a) significance in another dimension of the study, b) inclusion in a common pathway with a highly significant compound, or c) residing in a similar functional biochemical family with other significant compounds. Refer to the Statistical Methods and Terminology section of the Appendix for general definitions and further descriptions of false discovery rate and other statistical tests used at Metabolon.

Tables 13A-E show summary tables for the statistical tests performed in the study.

TABLE 13A
ANCOVA: Adjusted for diabetes status and tobacco use (part 1)
Statistical Comparisons, Volume Normalization

| | GROUP NAME | DIABETES2 | TOBACCO2 |
|---|---|---|---|
| | MAIN EFFECT | MAIN EFFECT | MAIN EFFECT |
| | (INFECTION vs. | (DIABETES vs. | (TOBACCO vs. |
| ANCOVA | CONTROL) | NO DIABETES) | NO TOBACCO) |
| Total | 232 | 140 | 86 |
| biochemicals | | | |
| p ≤ 0.05 | | | |
| Total | 116 | 87 | 57 |
| biochemicals | | | |
| 0.05 < p < 0.10 | | | |

TABLE 13B
ANCOVA: Adjusted for diabetes status and tobacco use (part 2)
Statistical Comparisons, Volume Normalization

| | INFECTION |
|---|---|
| ANCOVA Contrasts | CONTROL |
| Total biochemicals | 232 |
| p ≤ 0.05 | |
| Biochemicals (↑↓) | 100 \| 132 |
| Total biochemicals | 116 |
| 0.05 < p < 0.10 | |
| Biochemicals (↑↓) | 51 \| 61 |

TABLE 13C
Comparison of Positive and Negative culture samples
Statistical Comparisons, Volume Normalization

| | CULTURE_POS_1 |
|---|---|
| Welch's Two-Sample t-Test | CONTROL_POS_0 |
| Total biochemicals | 110 |
| p ≤ 0.05 | |
| Biochemicals (↑↓) | 55\| 55 |
| Total biochemicals | 73 |
| 0.05 < p < 0.10 | |
| Biochemicals (↑↓) | 45 \| 28 |

TABLE 13D
Welch's t-test: Comparison of Monomicrobial
(M1) and Polymicrobial (M2) culture samples
Statistical Comparisons, Volume Normalization

| | M2 |
|---|---|
| Welch's Two-Sample t-Test | M1 |
| Total biochemicals | 57 |
| p ≤ 0.05 | |
| Biochemicals (↑↓) | 36\| 21 |
| Total biochemicals | 50 |
| 0.05 < p < 0.10 | |
| Biochemicals (↑↓) | 21\| 29 |

TABLE 13E
Matched pairs t-test: Comparison of paired infection and control samples
Statistical Comparisons, Volume Normalization

| | INFECTION |
|---|---|
| Matched Pairs t-Test | CONTROL |
| Total biochemicals | 248 |
| p ≤ 0.05 | |
| Biochemicals (↑↓) | 103\| 145 |
| Total biochemicals | 114 |
| 0.05 < p < 0.10 | |
| Biochemicals (↑↓) | 48\| 66 |

### Biological Interpretation

Fracture-related infections (FRIs) are a significant complication in orthopedic trauma. The most common site of occurrence is the location of the bone fracture, often associated with surgical implants. These infections can lead to delayed healing, chronic osteomyelitis, and in severe cases, loss of function or amputation. The most common causative agents include the Gram-positive bacteria Staphylococcus aureus and Staphylococcus epidermidis. Other bacteria, including Gram-negative organisms, can also be involved. Open fractures, surgical intervention, poor health or immune status, and the use of fixation devices (both internal and external) are the most common risk factors. Diagnosis of FRI relies on several techniques, including evaluation by physicians, laboratory markers (blood cell counts, C reactive protein, etc.), imaging, and microbiological culture tests. The goal of this study was to investigate if biomarkers can be identified in the blood of fracture patients that are predictive of whether the patient goes on to have an infection. With this objective in mind, the investigator submitted plasma samples from patients with confirmed FRI for metabolomic profiling along with samples from a control cohort which were selected to match the FRI samples based on age, fracture location, and time after surgery.

### General Considerations for Data Interpretation

Data sets provided in the Data+Interpretation product can be quite large and contain a great deal of information. A few observations are offered below as an initial overview of metabolomic changes observed in the various groups. This report first presents statistical analysis of the experimental samples by various methods, including principal component analysis, followed by a discussion of the metabolic pathways and biochemicals that differed between the various groups. These are not presented as a comprehensive analysis; the PI, with a much greater knowledge of the experimental system, is encouraged to make a detailed study of the data for additional or alternative interpretations. For convenience, biochemicals are highlighted in bold text when they correspond to data plots or heatmap figures shown in the report and accompanying Graphics file.

Metabolon recommends a group size of 25+ replicates for human studies to capture the biological variability within datasets. In the case of this study, the main comparison of Infection versus Control contained adequate group sizes. However, for some of the potential covariates such as diabetes status, implant, NSAIDs, tobacco (among others), the group sizes become small enough to limit the statistical significance and hence biochemical interpretation of this study. Also included are comparisons for some of the sub-groups with larger sizes, such as mono vs. poly cultures and Culture positive vs. Culture negative samples.

### Data Overview

### Principal Component Analysis (PCA)

Principal component analysis (PCA) is an unsupervised mathematical dimension reduction procedure that can be used to obtain a high-level view of large datasets by allowing a large set of variables to be represented as a smaller set of variables. It uses an orthogonal transformation to convert a set of observations of possibly correlated variables into a set of variables called principal components (PC). The method permits visualization of how individual samples in a dataset differ from each other and can aid in the identification of patterns and underlying trends in complex datasets. When the PCA components explaining the greatest amount of variation (PC1, PC2) are plotted, samples with similar biochemical profiles tend to cluster close together whereas samples with distinct profiles tend to segregate from one another.

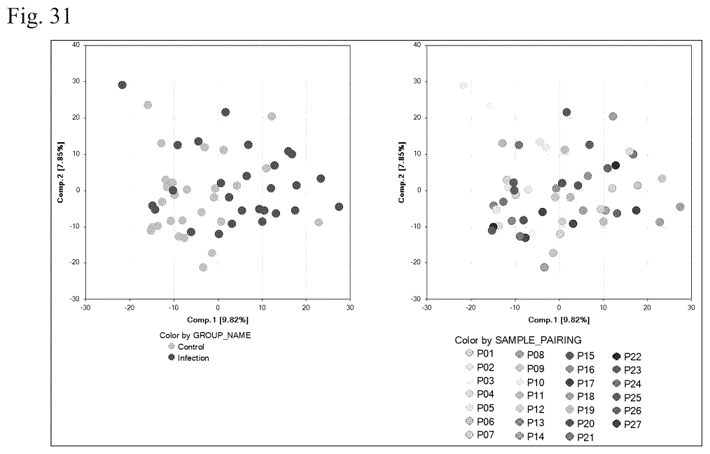

When applied to the plasma samples in this study, PCA revealed significant overlap among the Control and Infection groups, albeit with the Infection samples tending to plot more towards the right side of PC1 (FIG. 31). When evaluated based on sample matching criteria across cohorts, a range of patterns is observed with certain paired samples clustering closely (see P13, P16, and P18 among others), and others clustering at larger distance (see P03, P21, and P22). This is not unexpected since the pairing was based on individuals with similar demographics but still represent two distinct individuals with unique metabolomic profiles. These are dependent on many more variables than the matching demographic categories, whereas a repeated measures study from the same individual (which is obviously not a viable strategy for this study design) are expected to be very similar, with the differences accounted for nearly entirely by the experimental variable. The findings suggest that immediate plasma-derived metabolomic biomarkers may be able to indicate who is more likely to go on to infection, but that the specific signature still shows a relatively moderate effect.

### Statistical Summary

For this study, several statistical approaches were utilized. Firstly, differences between the infected and uninfected subjects were assessed using an ANCOVA model. Subject pairing was not considered for this model however tobacco use and diabetes status were considered as covariates for this analysis. Secondly, the infected and control cohorts were compared via a matched pairs t-test after taking into account the subject matching criteria provided by the investigator. Finally, Welch's two sample t-test were utilized to identify differences between samples stratified according to culture status (positive vs. negative) and mode of infection (monomicrobial vs. polymicrobial).

When evaluating these results, there are several considerations of note. Given that 1,453 metabolites were detected, up to 73 differences between groups may be expected to reach statistical significance by random chance alone if a cutoff value of p<0.05 is used. As shown in Tables 13A-E, only the Tobacco main effect in the ANCOVA analysis had somewhere near this value (86 differences). The Infection vs. Control pairwise comparison within the ANCOVA showed 232 significant differences, while the matched pairs t-Test for the same comparison showed 248 differences. Importantly, this is consistent with the Infection samples having distinct metabolomic profiles from the Control samples. Fewer differences were observed when comparing between samples based on culture status (110 differences) and mode of infection (55 differences). This suggests the type of infection (whether single or multiple species) does not play a significant role in influencing the metabolomic profile, though the limited group sizes should be taken into consideration when deriving conclusions from these tests.

### Random Forest Analysis (RFA)

Random forest analysis (RFA) is a supervised classification technique that can be useful for identifying possible biomarkers. Briefly, with this method the metabolomic profiles from a random subset of samples is used to generate a decision tree that is then tested on the remaining samples to see how well they can predict experimental groupings, with the process repeated multiple tens of thousands of times. Predictive ability of the aggregated and ranked results above that expected from random chance alone indicates that the experimental groups being compared can be effectively distinguished based on their metabolic profiles. A list of the top biochemicals that aid in correct group assignment can also be generated. For this analysis, the matched status of the samples was also taken into account. The overall predictive ability of the model was ˜80%, whereas 50% would be expected from random group assignment (FIG. 22). That value (80%) is at the lower end of what could be described (as an arbitrarily chosen cutoff) as ‘good’ predictive ability but is well above the level of random chance. The top 30 predictive metabolites in the analysis consisted of lipids (11), amino acid and xenobiotic related metabolites (6 each), and unknowns (5).

### Observed Biochemical Differences

### Fatty Acid Metabolism

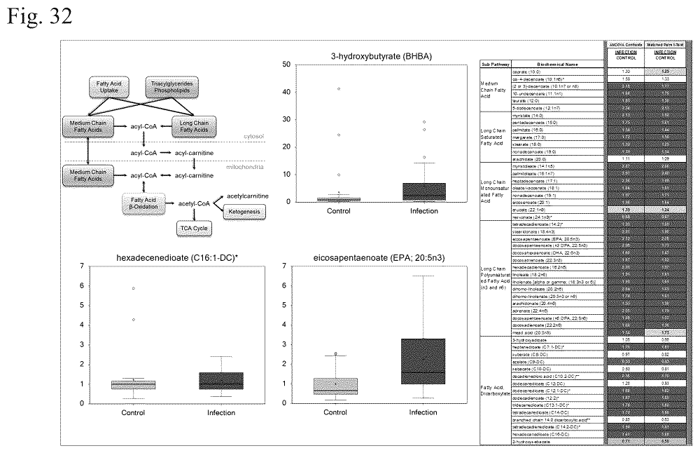

When cellular free fatty acids (FFA) are in excess of the cells ability to utilize them in 3-oxidation or complex lipid assembly, acylcarnitines can cross the cellular membrane to be exported to the bloodstream. The enzyme carnitine palmitoyl transferase (CPT1) exchanges carnitine for CoA on fatty acids to generate acylcarnitines and thus permit the movement acyl-chains across the mitochondrial membrane, to facilitate fatty acid β-oxidation. FFA can also be generated from phospholipid cleavage of acyl chains. In times of stress or energy demand beyond carbohydrate availability, FFA are used in b-oxidation to generate acetyl-CoA for input into the TCA cycle for oxidative phosphorylation. In this dataset, the Infection group was found to have elevated levels of medium and long chain fatty acids, including saturated, monounsaturated, and polyunsaturated derivatives (eicosapentaenoate (EPA; 20:5n3) is shown; see FIG. 32). Dicarboxylic fatty acids (hexadecanedioate (C16:1-DC)) were also observed to be elevated. The lipid species are produced by fatty acid w-oxidation, which can serve as a rescue pathway when b-oxidation is disrupted or overwhelmed. Interestingly, while some acylcarnitines are also elevated, there are many fewer statistically significant differences. Additionally, acetylcarnitine and the ketone bodies acetoacetate and 3-hydroxybutyrate (BHBA) are elevated, consistent with excess acetyl-CoA production. The findings may suggest that lipolysis, fatty acid oxidation, and ketone body production were elevated in the patients who went on to develop infection at the fracture site. Importantly and dependent on the timing of the blood collection with respect to the injury, this pattern may also could have been influenced by a lack of normal eating patterns due to the fracture event. Alternatively, the increase in FFAs may be a direct result of the injury as an acute reaction to any tissue damage associated with the break.

### Phospholipid Metabolism

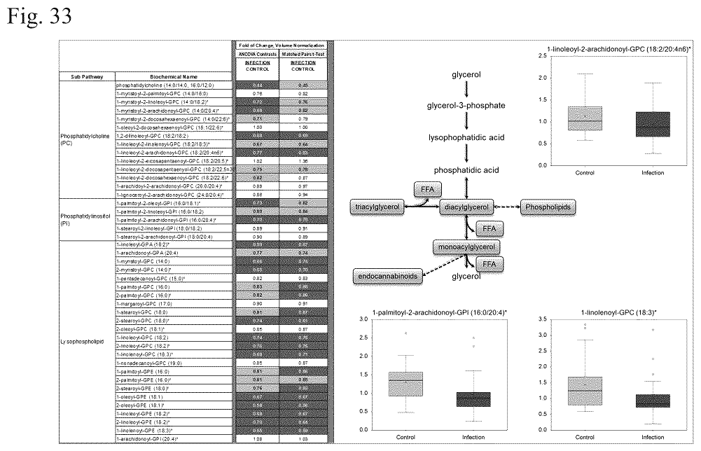

Phospholipids make up the largest proportion of cell membrane lipids and proliferating cells synthesize new phospholipids from diacylglycerols and polar head groups such as choline or ethanolamine. Phosphatidylcholine and phosphatidylethanolamine make up the largest proportion of membrane phospholipids. FFAs (discussed above) are also generated from lipase cleavage of phospholipids with the release of a free fatty acid. In this set of data, few if any of the main phospholipid precursors such as choline, ethanolamine, or glycerophosphocholine (not a complete list) are significantly altered between Infection and Control groups. However, multiple phosphatidylcholine (PC) lipids (1-linoleoyl-2-arachidonoyl-GPC (18:2/20:4n6)), phosphatidylinositol (PI) lipids (1-palmitoyl-2-arachidonoyl-GPI (16:0/20:4)), and lysophospholipids (1-linolenoyl-GPC (18:3)) are significantly decreased in the Infection group compared to Control (FIG. 33). This might reflect a lower overall phospholipid membrane biosynthesis/remodeling state in the individuals that went on to get an FRI. Speculating, it can be envisioned that the tissue surrounding the fracture site for those that do not get an infection was more metabolically active, giving rise to higher levels of phospholipid associated lipids in the blood, possibly leading to better immune clearance of the invading microorganism. It is also possible the lower levels in the Infection group present a readout of the lack of response to the fracture related tissue damage, leading to overall lower levels of PL metabolites. In this scenario, the Control group (i.e., no infection) would be predicted to show a robust response to the tissue damage caused by the bone break, and therefore as elevated levels of the PL intermediates necessary for proper tissue regeneration.

Also of note, in the Culture positive vs. Culture negative comparison, ceramides and sphingomyelins are lower in the Infection group. Sphingomyelins are a class of phospholipids that are found in all animal cell membranes. They are structurally distinct from the other phospholipids in that they lack the glycerol backbone that is found in glycerophospholipids. Instead, they are composed of ceramide (a lipid made up of sphingosine and a fatty acid) and a polar head group (phosphocholine in most cases). The unique composition of sphingomyelins allows them to play important roles in both membrane stabilization and lipid signaling as a part of lipids rafts. The decrease in the sphingomyelin and ceramide pool of the Culture positive group may be influenced by several factors, including fatty acid availability, membrane turnover, and/or uptake of circulating PL species. This may be supportive of pathologies of infection that occur due to culturable vs. unculturable species of microorganisms.

### Diet and Microbiome Related Metabolites

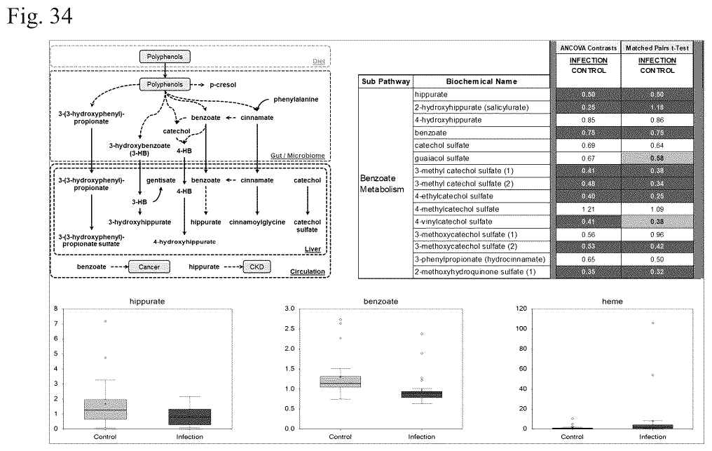

Biochemicals derived from the catabolism of aromatic amino acids and compounds associated with the digestion of plant components, such as cell wall flavonoids and lignin, serve as useful markers of the microbiome since the digestion of aromatic amino acids and plant compounds depends, in part, on reactions catalyzed by microbe genome-encoded enzymes. In this dataset, a few aromatic amino acid metabolites were altered, and in particular several benzoates, xanthines, and other Plant/Food related metabolites were decreased in the Infection group (FIG. 34; hippurate, benzoate). The metabolism of benzoate, which can be derived from aromatic compounds, polyphenols and purines (and also food as a preservative), has a substantial contribution from the gut microbiome. Differences in benzoate levels and its metabolites in the infected and control groups may be indicative thus of a base level difference in the metabolism of these types of biochemicals, which would likely be directly related to the microbiome state/status of the individual. Of note, in the random forest analysis, five (5) xenobiotic metabolites were identified among the top 30 biomarker candidates, with benzoate as the top candidate. This suggests that whatever the cause/correlation, microbiome associated metabolites are indeed a possible indicator of future infection. Similar decreases in xanthines (also primarily diet-derived) and other Food related metabolites (such as vitamins and other cofactors) also are observed, which (as discussed above for FFA), could be related to dietary differences/changes occurring because of the fracture. Potentially of interest within this group, heme, the iron-based co-factor critical for proper red blood cell (RBC) function, was elevated in the Infection group (albeit possibly because of 2 samples with very high levels). 2,3-diphosphoglycerate shows a similar pattern. This is a red blood cell specific metabolite, which may suggest increased hemolysis in the infection samples. Interestingly, when heme is broken down, it is first converted into biliverdin and bilirubin (via heme oxygenase and biliverdin reductase activities). It is notable that neither of these compounds was significantly altered. This may suggest that elevated heme and 2,3-DPG present a readout of RBC damage caused by the fracture, but that overall heme metabolism is not greatly affected at the time of the plasma sampling.

### Bile Acid Metabolism

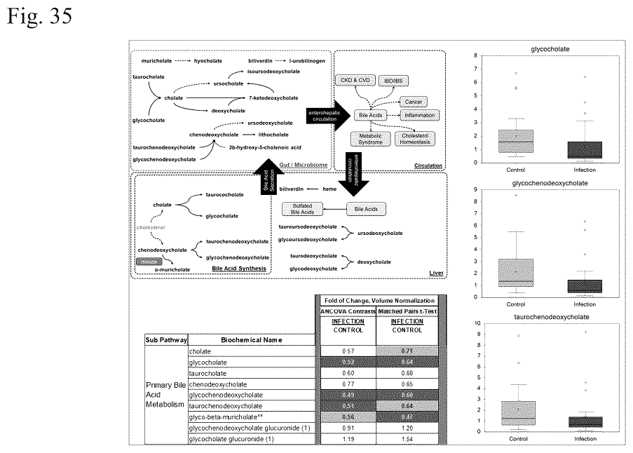

Bile acids are steroid acid derivatives of cholesterol that function as important physiological surfactants. They facilitate the elimination of cholesterol, emulsification of dietary fats (aiding their absorption), and clearance of hepatic catabolites (e.g., bilirubin) that are both hydrophobic and hydrophilic in nature. They are intricately involved in cellular signaling, gut metabolism, and the composition of the microbiome (PMID: 34127070). Primary bile acids are synthesized from cholesterol as a precursor in the liver and can also be conjugated to glycine or taurine within the liver. The primary bile acids are secreted into the duodenum and travel with digested food material through the small intestine, where ˜95% are reabsorbed in the ileum and transported back to the liver via enterohepatic circulation (EHC). Some primary bile acids can be structurally modified by gut bacteria in the lower gastrointestinal tract into secondary bile acids (PMID: 34127070). Microbial metabolism of primary bile acids is so prevalent that most fecal excreted bile acids are secondary bile acids, even though primary bile acids are exclusively generated by the host. Interestingly, bile acids can act as antimicrobial agents and can impact susceptible bacteria in both a bactericidal and bacteriostatic manner (PMID: 34127070). This last point is especially of interest given that the current dataset shows that primary bile acids are lower in the infection group (glycocholate, glycochenodeoxycholate, taurochenodeoxycholate) compared to the Control group (FIG. 35). The above noted differences in benzoate/diet-related biochemicals may be related to these changes, or they may be readout of inherent differences between the Infection and Control groups. The lower levels found in the Infection samples could be indicative of overall lower levels of bile acid production or absorption, which may be modulating the ability of microbes to colonize fracture sites.

This set of plasma samples produced a robust set of >1400 metabolites for analysis. By principal component analysis, a moderate grouping of Infection and Control samples was evident, with not insignificant overlap between the two populations. A matched pairs random forest analysis produced an overall predictive ability of ˜80% when comparing Infection vs. Control samples, with roughly similar mis-identified samples in each group (18.5% error for Control group vs. 22.2% error for Infection group). The most abundant biochemicals types in the top 30 metabolites aiding in group distinction were lipids, xenobiotics, and amino acid related metabolites. When looking at the distinct biochemical pathways that differ between Infection and Control samples, lipid metabolism (free fatty acids including dicarboxylic fatty acids; phospholipid species), benzoates/plant and food components/xanthines, and bile acids were all affected by the infection status of the samples. Additionally, sphingolipids and ceramides were altered in the Culture positive vs. Culture negative samples. Moving forward, comparison of plasma profiles at intermediates time between the initial and return collection may be able to pinpoint the precise role of certain metabolites in the infection process. Additionally, increasing group sizes (if possible) for site of fracture and type of fracture may help with identifying a greater number of metabolites that are involved in infection for different types of wounds. In this respect, it is of note that the lactoyl amino acids show a consistent decrease in levels in the infection group. These may also be presenting some type of readout of plasma amino acid regulation, although they are a relatively new class of metabolites with currently unclear biology.

### Study Parameters

### Data Quality: Instrument and Process Variability

TABLE 14
Values for instrument and process variability

| Quality Control Sample | Median RSD |
|---|---|
| (MTRX7) | Plasma |
| Internal Standards | 4% |
| Endogenous Biochemicals | 8% |

Instrument variability was determined by calculating the median relative standard deviation (RSD) for the internal standards that were added to each sample prior to injection into the mass spectrometers. Overall process variability was determined by calculating the median RSD for all endogenous metabolites (i.e., non-instrument standards) present in the MTRX7 technical replicates. Values for instrument and process variability meet Metabolon's acceptance criteria as shown in the table above.

### Platform

For experimental details regarding sample accessioning, sample preparation, QA/QC, Ultrahigh Performance Liquid Chromatography-Tandem Mass Spectroscopy (UPLC-MS/MS), bioinformatics, LIMS, data extraction and compound identification, compound quality control, and metabolite quantification and data normalization, see the Supplementary Content 1 section above.

1. A method of treating a fracture-related infection (FRI) in a subject having FRI, said method comprising:
analyzing a subject's biological sample using an analytical method to determine whether a subject has an FRI; and
administering an anti-infection therapy to said subject identified as having an FRI.

2. The method of claim 1, wherein the analytical method provides an analytical patient profile.

3. The method of claim 1, wherein the biological sample comprises blood, urine, a synovial fluid, or a combination thereof.

4. The method of claim 1, wherein the biological sample comprises blood plasma.

5. The method of claim 1, wherein the biological sample comprises a biomarker.

6. The method of claim 5, wherein the biomarker comprises a protein, a non-protein metabolite, a xenobiotic, heme, a nucleic acid, or any combination thereof.

7. The method of claim 6, wherein the biomarker comprises a lipid, a sugar, a beta hydroxy acid, an amino acid, a bile acid, or a derivative thereof.

8. The method of claim 7, wherein the biomarker comprises myristoleate, 14-methylpalmitate, 15-methylpalmitate, 16-hydroxypalmitate, 10-undecenoate, palmitoleate, 12-methylmyristate, 13-methylmyristate, 5-dodecenoate, pentadecanoate, myristate, glycholate, hexadecadienoate, eicosapentaenoate, hexadecanedioate, 1-linoleoyl-2-arachidonoyl-GPC (18:2/20:4n6), 1-palmitoyl-2-arachidonoyl-GPI, -linolenoyl-GPC (18:3), or any combination thereof.

9. The method of claim 7, wherein the amino acid or a derivative thereof comprises alanine, N-lactoyl-isoleucine, N-lactoyl-tyrosine, N-acetyl-glutamide, methionine sulfoxide, or any combination thereof.

10. The method of claim 6, wherein the biomarker comprises benzoate, vanilloylglycine, 3-hydroxy-2-methylpyridine sulfate, hippurate, 2-hydroxyhippurate (salicylurate), levulinate (4-oxovalerate), ferulic acid 4-sulfate, vanillic alcohol, a derivative thereof, or any combination thereof.

11. The method of claim 6, wherein the biomarker comprises alpha-1-acid glycoprotein 1, serum amyloid A-2 protein, leucine-rich alpha-2-glycoprotein, complement factor H-related protein 5, C-reactive protein (CRP), complement component C9, haptoglobin, serum amyloid A-1 protein, lipopolysaccharide-binding protein, peptidyl-prolyl cis-trans isomerase A, calmodulin-3, fructose-bisphosphate aldolase A, complement factor B, peroxiredoxin-2, mannosyl-oligosaccharide 1,2-alpha-mannosidase IA, alpha-1-acid glycoprotein 2, Transgelin-2, complement C1r subcomponent, retinoic acid receptor responder protein 2, ceruloplasmin, complement C1s subcomponent, carboxypeptidase N catalytic chain, alpha-2-macroglobulin, coagulation factor X, Insulin-like growth factor-binding protein 4, selenoprotein P (SEPP1), fibronectin, cell surface glycoprotein MUC18, hepatocyte growth factor activator, apolipoprotein A-IV, plasma kallikrein, afamin, platelet-derived growth factor AB/BB (PDGF-AB/BB), monokine induced by gamma interferon (MIG), Interleukin 6 (IL-6), vascular endothelial growth factor A (VEGF-A), fibrin, procalcitonin, neutrophil CD64 (nCD64), CD66b, or any combination thereof.

12. The method of claim 1, wherein the step of analyzing a subject's biological sample comprises:
obtaining an analytical patient profile of a biological sample obtained from the subject; and
comparing the analytical patient profile to a reference analytical patient profile.

13. The method of claim 12, wherein the step of comparing the analytical patient profile to a reference analytical patient profile comprises determining if the biological sample comprises an increased amount or a decreased amount of a biomarker compared to a reference analytical patient profile.

14. The method of claim 12, wherein the analytical method comprises a mass spectrometer or a Fourier-transform infrared spectroscopy (FTIR) spectrometer.

15. The method of claim 14, wherein the analytical method comprises Tandem mass tag liquid chromatography-mass spectrometry (TMT LC-MS/MS), a reverse phase ultrahigh performance liquid chromatography-tandem mass spectroscopy (RP)/UPLC-MS/MS method using positive ion mode electrospray ionization (ESI), or a (RP)/UPLC-MS/MS method using negative ion mode ESI.

16. The method of claim 1, wherein the FRI is associated with a surgical implant; or the introduction of an implant, the replacement of an implant, or the adjustment of an implant.

17. The method of claim 1, wherein the FRI is caused by one or more pathogens.

18. The method of claim 17, wherein the pathogen comprises a Gram-positive bacterium, a Gram-negative bacterium, an anaerobic bacterium, a mycobacterium, a fungus, or any combination thereof.

19. The method of claim 18, wherein the Gram-positive bacterium comprises Staphylococcus aureus, Staphylococcus epidermidis, or a combination thereof.

20. The method of claim 18, wherein the Gram-negative bacterium comprises Pseudomonas aeruginosa, Escherichia coli, or a combination thereof.
